(19) United States
(12) Patent Application Publication
(10) Pub. No.: US 2016/0139120 A1
(43) Pub. Date: May 19, 2016

(54) RAGE AS A C1Q RECEPTOR, METHODS AND APPLICATIONS

(71) Applicants: Gaetano R. BARILE, (US); Ann Marie SCHMIDT, (US); Wanchao MA, (US)

(72) Inventors: Gaetano R. BARILE, River Vale, NJ (US); Ann Marie SCHMIDT, Franklin Lakes, NJ (US); Wanchao MA, New York, NY (US)

(73) Assignee: The Trustees of Columbia University in the City of New York, New York, NY (US)

(21) Appl. No.: 14/767,561

(22) PCT Filed: Feb. 12, 2014
(86) PCT No.: PCT/US14/16137

§ 371 Date: Aug. 12, 2015

Related U.S. Application Data
(60) Provisional application No. 61/763,741, filed on Feb. 12, 2013.

(51) Int. Cl. G01N 33/566; C12N 15/113; C07K 16/28

(52) U.S. Cl. CPC G01N 33/566; C07K 16/28; C12N 15/1138; G01N 2500/02; G01N 2333/70503; C12N 2310/16; C12N 2310/14

(57) ABSTRACT

A method of identifying a compound capable of reducing or increasing the amount of the receptor for RAGE-C1q Binding comprising a) providing an amount of the compound to be tested; b) contacting the amount of the compound with C1q and RAGE; c) measuring the amount of RAGE-C1q binding in step b); d) comparing the amount of RAGE-C1q binding measured in step c) with the amount of RAGE-C1q binding measured under corresponding conditions in the absence of the compound; and e) identifying the compound as capable of reducing or increasing the amount of RAGE-C1q binding. The present invention also provides a method of treating a subject suffering from a RAGE-related disorder comprising administering to the subject a therapeutically effective amount of a compound that is a modulator of the amount of RAGE-C1q binding so as to thereby treat the subject.

This application claims priority of U.S. Provisional Application No. 61/763,741 filed Feb. 12, 2013, the entire contents of which are hereby incorporated by reference.

Throughout this application, various publications are referred to by first author and year of publication. Full citations for these publications are presented in a References section immediately before the claims. Disclosures of the publications cited in the References section in their entireties are hereby incorporated by reference into this application in order to more fully describe the state of the art as of the date of the invention described herein.

### BACKGROUND OF INVENTION

### Receptor for Advanced Glycation Endproducts (RAGE)

The cellular receptor RAGE is a multiligand signal transduction receptor of the immunoglobulin superfamily of cell surface molecules that mediates a spectrum of distinct cell-specific effects in settings characterized by accumulation of its ligands. (Ramasamy, 2005). The gene encoding RAGE is located within the major histocompatibility complex (MHC) class III region on chromosome 6. The receptor is a type I membrane protein with 5 domains: an extracellular structure composed of one variable (V)-type immunoglobulin (Ig) domain followed by two distinct constant (C)-type domains; a single transmembrane spanning domain; and a short, highly negatively-charged cytoplasmic tail that is essential for RAGE-mediated signal transduction. (Schmidt, 2001)

The cellular receptor RAGE was first identified as a receptor for advanced glycation endproducts (AGES), leading to its nomenclature. AGEs are heterogeneous group of adducts of nonenzymatic glycation and oxidation of amino groups of proteins and lipids that develop during normal metabolic aging. AGEs form in multiple milieu, including aging, hyperglycemia, oxidant stress, inflammation, neurodegenerative disorders, and renal failure, and accumulate more rapidly in the settings of hyperglycemia, oxidant stress, and inflammation. (Ramasamy, 2005)

While initially identified as a receptor capable of mediating AGE-related cellular perturbations, RAGE can bind several ligands and is now considered a multiligand, pattern-recognition receptor.

(Ramasamy, 2005) Well-characterized ligands for RAGE include: AGEs (particularly carboxymethyl-lysine [CML]-protein adducts), HMGB1 (high mobility group box 1) or amphoterin, the S100/calgranulin family of cytokine-like mediators, amyloid-β peptide (Aβ), and the leukocyte counterreceptor Mac-1 (integrin CD11b/CD18).

Upregulation of RAGE and its ligands trigger a series of cellular responses, including but not limited to the neurite outgrowth during development, cell proliferation and spread in cancer, initiation and propagation of inflammatory responses in diabetes, and neuronal toxicity in Alzheimer's disease. Biological effects of RAGE are related to its ability to activate intracellular transduction pathways, and numerous, diverse signalling cascades have been identified in the setting of ligand/RAGE activation. (Ramasamy, 2005)

### The Complement Protein C1q

C1q is the target recognition protein of the classical complement pathway and a major connecting link between innate and acquired immunity.

Several functions have been assigned to C1q, which include antibody-dependent and independent immune functions. (Duus, 2010; Zutter, 2007; Ghiran, 2002). C1q-immune complex interactions are clearly important, but C1q still retains significant antibody-independent opsonic and other functions as well. These antibody-independent functions are partially attributed to C1q receptors present on the effector cell surface. (Duus, 2010; Zutter, 2007; Ghiran, 2002). There remains uncertainty about the precise identities of receptors that mediate C1q functions. Some previously described C1q receptor molecules, such as gC1qR and cC1qR, now appear to have a less prominent role in C1q function compared to those unrelated to C1q. (McGreal, 2002) It has also been shown that C1q is involved in the selective elimination of inappropriate synaptic connections during neural circuit development, and similar processes may become aberrantly reactivated in neurodegenerative disease. These findings support a model in which unwanted synapses are tagged by C1q for elimination. (Stevens, 2007)

### SUMMARY OF THE INVENTION

RAGE, the multiligand receptor of the immunoglobulin superfamily of cell surface molecules, is implicated in innate and adaptive immunity. Complement component C1q serves roles in complement activation and antibody-independent opsonization. Disclosed herein is a finding that RAGE is a native C1q globular domain receptor.

The subject invention provides a method of identifying a compound capable of inhibiting binding of receptor for advanced glycation endproduct (RAGE) with Complement C1q (C1q) (RAGE-C1q Binding) comprising:
a) providing an amount of the compound to be tested;
b) contacting the amount of the compound with
   i) an amount of C1q, and then combining the amount of C1q with an amount of RAGE under conditions that permit RAGE-C1q binding,
   ii) an amount of RAGE, and then combining the amount of RAGE with an amount of C1q under conditions that permit RAGE-C1q binding, or
   iii) a mixture of an amount of C1q and an amount of RAGE under conditions that permit RAGE-C1q binding;
c) measuring the amount of RAGE-C1q binding in step b);
d) comparing the amount of RAGE-C1q binding measured in step c) with the amount of RAGE-C1q binding measured under corresponding conditions in the absence of the compound; and
e) identifying the compound as capable of inhibiting RAGE-C1q binding if the amount of RAGE-C1q binding measured in step c) is less than the amount of RAGE-C1q binding in the absence of the compound under corresponding conditions.

The subject invention also provides a method of identifying a compound capable of increasing RAGE-C1q binding comprising:
a) providing an amount of the compound to be tested;
b) contacting the amount of the compound with
   i) an amount of C1q, and then combining the amount of C1q with an amount of RAGE under conditions that permit RAGE-C1q binding,
   ii) an amount of RAGE, and then combining the amount of RAGE with an amount of C1q under conditions that permit RAGE-C1q binding, or
   iii) a mixture of an amount of C1q and an amount of RAGE under conditions that permit RAGE-C1q binding;
c) measuring the amount of RAGE-C1q binding in step b);
d) comparing the amount of RAGE-C1q binding measured in step c) with the amount of RAGE-C1q binding measured under corresponding conditions in the absence of the compound; and
e) identifying the compound as capable of increasing RAGE-C1q binding if the amount of RAGE-C1q binding measured in step c) is more than the amount of RAGE-C1q binding in the absence of the compound under corresponding conditions.

The subject invention also provides a method of reducing phagocytosis by a phagocyte comprising contacting the phagocyte with an effective amount of a compound capable of inhibiting RAGE-C1q binding, thereby reducing phagocytosis by the phagocyte.

The subject invention also provides a method of treating a subject suffering from a disease associated with an increase of RAGE expression comprising administering to the subject a therapeutically effective amount of a compound capable of inhibiting RAGE-C1q binding so as to thereby treat the subject.

The subject invention also provides a method of treating a subject suffering from a disease associated with a decrease of RAGE expression comprising administering to the subject a therapeutically effective amount of a compound capable of increasing RAGE-C1q binding so as to thereby treat the subject.

The subject invention also provides a method of treating a subject in need of treatment of an inflammatory disease, comprising administering to the subject a therapeutically effective amount of a compound capable of inhibiting RAGE-C1q binding on the surface of white blood cells (leukocytes) present in the subject, thereby treating the subject.

The subject invention also provides a method of treating a subject in need of treatment of an inflammatory disease, comprising administering to the subject a therapeutically effective amount of a compound capable of increasing RAGE-C1q binding on the surface of white blood cells (leukocytes) present in the subject, thereby treating the subject.

The subject invention also provides a method of treating a subject in need of treatment of a disease associated excess apoptosis, comprising administering to the subject a therapeutically effective amount of a compound capable of inhibiting RAGE-C1q binding on the surface of white blood cells (leukocytes) present in the subject, thereby treating the subject.

The subject invention also provides a method of treating a subject in need of treatment of a disease associated with a reduction of apoptosis, comprising administering to the subject a therapeutically effective amount of a compound capable of increasing C1q-RAGE binding on the surface of white blood cells (leukocytes) present in the subject, thereby treating the subject.

The subject invention also provides a method of strengthening an immune system response against a bacterial infection and bacterial cells, comprising increasing RAGE-C1q binding between 1) C1q bound to bacterial cells and 2) RAGE, thereby strengthening the immune system response against bacterial infection and bacterial cells.

The subject invention also provides a kit for use in identifying a modulator of RAGE-C1q binding comprising:
a) RAGE;
b) C1q; and
c) A candidate Modulator of RAGE-C1q binding.

The subject invention also provides a method of identifying a compound as a modulator of RAGE-C1q binding comprising
a) providing an amount of the compound to be tested;
b) contacting the amount of the compound with
   i.) an amount of C1q, and then combining the amount of C1q with an amount of RAGE under conditions that permit RAGE-C1q binding,
   ii.) an amount of RAGE, and then combining the amount of RAGE with an amount of C1q under conditions that permit RAGE-C1q binding, or
   iii.) a mixture of an amount of C1q and an amount of RAGE under conditions that permit RAGE-C1q binding;
c) measuring the amount of RAGE-C1q binding in step b);
d) comparing the amount of RAGE-C1q binding measured in step c) with the amount of RAGE-C1q binding measured under corresponding conditions in the absence of the compound; and
e) identifying the compound as a modulator of RAGE-C1q binding, if the amount of RAGE-C1q binding measured in step c) is different than the amount of RAGE-C1q binding in the absence of the compound under corresponding conditions.

The subject invention also provides a method of treating a subject suffering from a RAGE-related disorder comprising administering to the subject a therapeutically effective amount of a compound that is a modulator of RAGE-C1q binding so as to thereby treat the subject.

### BRIEF DESCRIPTION OF THE SEVERAL VIEWS OF THE DRAWINGS

FIG. 1. Comparison of Avastin (human IgG1) and sRAGE on complement activation. The experiment was carried out similarly as described in FIG. 14. sRAGE has 1/8 activity of Avastin upon complement activation.

FIG. 2. Immunofluorescence studies of 5100 protein in old monkey and human retina with drusen. 7A is a retina section that was prepared from 23-24 years old female rhesus monkey with moderately severe drusenoid maculopathy, 7B is a retina section from a 53 years old man with type II diabetes. All of the sections were immunostained with 5100 antibody and Alexa Fluor 488-labeled secondary antibody. Green signal is S100 protein and the red signal in RPE cells is autofluorescence.

FIG. 3. Co-localization of RAGE and Mac-1 on plasma membrane of activated U-937 cells.

U-937 cells (2×10⁵/ml) cultured in RPMI medium containing 10% fetal bovine serum, 100 units/ml of penicillin and 100 μg/ml of streptomycin were incubated with 100 nM of 1,25-dihydroxyvitamin D₃ (D₃) and 2 ng/ml of transforming growth factor-β (TGF-β) at 37° C. in a 5% CO₂ incubator for 3 days with D₃ and TGF-β refreshments everyday. After fixation of cells with 4% paraformaldehyde, immunofluorescence staining was performed with chicken anti-human RAGE IgY and mouse anti-human CD11b monoclonal IgG1. Secondary antibodies labeled with Alexa Fluor 546 and 488 were used for microscopy of RAGE and Mac-1 respectively.

FIG. 4. RAGE and Mac-1 dependent U-937 cells adhesion to C1q-coated plate.

U-937 cells (2×10⁵/ml) cultured in RPMI medium containing 10% fetal bovine serum, 100 units/ml of penicillin and 100 μg/ml of streptomycin were incubated with 100 nM of D₃ and 2 ng/ml of TGF-β at 37° C. in a 5% CO₂ incubator for 24 hours. After washing twice with serum-free RPMI medium, U-937 cells were suspended at concentration of 2×10⁵/ml in serum-free RPMI medium containing 0 or 100 μg/ml of chicken anti-human RAGE IgY or 20 μg/ml of F(ab′)₂ of mouse anti-human CD11b IgG1. As controls, 100 μg/ml of chicken anti-AGE IgY and 100 μg/ml of mouse IgG1 isotype control were used. 0.1 ml of cell suspension was added into 96-well MaxiSorp plate, which had been coated with 0.065 ml of 30 μg/ml of human C1q in PBS overnight at 4° C. and blocked with 0.1 ml of 10% of fetal bovine serum in PBS at 25° C. for 3 hours. The cells were cultured for 24 hours, then 50 μl of serum-free RPMI medium containing 15 μM of Calcein AM was added into each well to label U-937 cells with fluorescence for 1 hour. After three gentle washes with Hanks' balanced salt solution, the attached U-937 cells were photographed with regular light and fluorescent light, as shown in panel A. To quantitatively measure cell attachment, the plate was read with fluorescence plate reader with excitation at 485 nm and emission at 530 nm, as shown in panel B. The p value was calculated by student T-test with at least 4 samples for each condition.

FIG. 5. RAGE and Mac-1 dependent phagocytosis of C1q-coated beads by activated U-937 cells

U-937 cells (2×10⁵/ml) cultured in RPMI medium containing 10% fetal bovine serum (FBS), 100 units/ml of penicillin and 100 μg/ml of streptomycin were incubated with 100 nM of D₃ and 2 ng/ml of TGF-E3 at 37° C. in a 5% CO₂ incubator for 24 hours. After washing twice with serum-free RPMI medium, 2×10⁵/ml of U-937 cells were suspended with 2×10⁸/ml of FBS-coated beads or C1q-coated beads in serum-free RPMI medium containing 0 or 100 μg/ml of chicken anti-human RAGE IgY or 20 μg/ml of F(ab′)₂ of mouse anti-human CD11b IgG1. As controls, 100 μg/ml of chicken anti-AGE IgY and 100 μg/ml of mouse IgG1 isotype control were used. The suspension of cells and beads was then incubated in a 5% CO₂ incubator at 37° C. for 4 hours. Free beads were washed off after 3 washes with cold Hanks' balanced salt solution. The phagocytosed beads, as shown in panel A, were quantified by fluorescence readings with excitation at 530 nm and emission at 620 nm and with cell number correction at 280 nm. The results were shown in panel B. The p value was calculated by student T-test with at least 4 samples for each condition.

FIG. 6. Effect of phenolphthalein bisphosphate on C1q binding (A) and complement activation (B) of sRAGE. (A) NUNC MaxiSorp high protein-binding capacity 96-well ELISA plate was coated with 100 μl of 20 μg/ml of sRAGE (from insect cells) in 50 mM carbonate-bicarbonate buffer, pH9.6, at 4° C. overnight; all wells were blocked with 3% of BSA in PBS for 2 hours at room temperature. The resulted wells were incubated with 0.1 ml of 2 μg/ml of C1q in 2% BSA/PBS solution with 0.05% Tween 20 and 0-2 mM of phenolphthalein bisphosphate at room temperature for 2 hours. Binding of C1q was detected with anti-human C1q:HRP conjugate and o-Phenylenediamine dihydrochloride as final development substrate.

(B) 2 μg of sRAGE (from insect cells) and 1 mM of phenolphthalein bisphosphate were incubated in 20 μl of fresh human serum for 1 hour at 37° C. in a 5% CO₂ incubator. Complement activation was monitored as C3a formation from proteolytic cleavage of C3 by ELISA kit.

FIG. 7. Effect of phenolphthalein bisphosphate on U-937 cells adhesion to C1q-coated plate.

U-937 cells (2×10⁵/ml) cultured in RPMI medium containing 10% FBS, 100 units/ml of penicillin and 100 μg/ml of streptomycin were incubated with 100 nM of D₃ and 2 ng/ml of TGF-β at 37° C. in a 5% CO₂ incubator for 24 hours. After washing twice with serum-free RPMI medium, U-937 cells were suspended at concentration of 2×10⁵/ml in serum-free RPMI medium containing 0 or 2 mM of phenolphthalein bisphosphate, 0.1 ml of cell suspension was added into 96-well MaxiSorp plate, which had been coated with 0.060 ml of 30 μg/ml of human C1q in PBS overnight at 4° C. and blocked with 0.1 ml of 10% of FBS in PBS at 25° C. for 3 hours. The cells were cultured for 24 hours, then 50 μl of serum-free RPMI medium containing 15 μM of Calcein AM was added into each well to label U-937 cells with fluorescence for 1 hour. After three gentle washes with Hanks' balanced salt solution, the attached U-937 cells were photographed with regular light and fluorescent light, as shown in panel A. To quantitatively measure cell attachment, the plate was read with fluorescence plate reader with excitation at 485 nm and emission at 530 nm, as shown in panel B. The p value was calculated by student T-test with at least 4 samples for each condition.

FIG. 8. Expression of RAGE and Mac-1 in differentiated U937 cells.

A: U937 cells were differentiated with 100 nM of vitamin D3 and 2 ng/ml of TGF-β1 for 3 days, with vitamin D3 and TGF-β1 refreshment everyday. (A) Expression of RAGE and CD11b was monitored from day 0 to day 3 by Western blot of total 12 μg proteins. (B) Immunofluorescence staining of RAGE and CD11b after 3 days differentiation of U937 cells. Immunofluorescence staining of RAGE (red) was performed with chicken anti-human RAGE IgY and Alexa Fluor 546 goat anti-chicken IgY. CD11b detection (green) was carried out with mouse anti-human CD11b monoclonal IgG1 and Alexa Fluor 488 goat anti-mouse IgG1. Immunofluorescence signals were absent if the primary antibodies were omitted.

FIG. 9. Involvement of RAGE and Mac-1 in phagocytosis of apoptotic Jurkat cells and C1q-opsonized beads by differentiated U937 cells.

(A) Representative dot-plots of different cell populations separated by FACS after 2 h phagocytosis of apoptotic Jurkat cells: top-left U937 cells without phagocytosis; top-right phagocytic U937 cells labeled with percentage of total U937 population; bottom-right apoptotic Jurkat cells. (B) FACS analysis of phagocytosis of apoptotic Jurkat cells and effect of anti-RAGE and anti-CD11b antibodies. Phagocytosis index was calculated as percentage of basal phagocytosis, which was the phagocytosis at 37° C. control conditions without C1q addition. (C) Combination of phase contrast and fluorescence microscopy of differentiated U937 cells after phagocytosis of FBS-coated and C1q-coated Latex beads. (D) Quantitative analysis of phagocytosis of Latex beads by fluorescence with excitation at 530 nm and emission at 620 nm. Results in (B) and (D) were mean±SD of three independent experiments. *p<0.01 in pair comparison indicated in the graph.

FIG. 10. Direct binding of human C1q to immobilized human sRAGE.

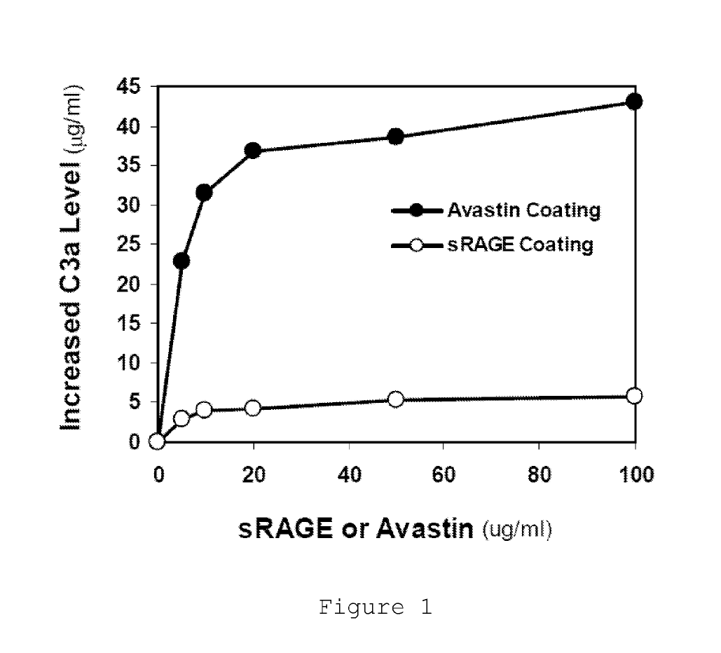

(A) and (B) were ELISA-like experiments to detect direct binding of C1q and sRAGE. MaxiSorp plates were coated with 20 μg/ml sRAGE. After blocking with BSA, the plates were incubated with 2 μg/ml of C1q (A) or 500-fold diluted fresh human serum (B) in PBS containing 2% BSA and 0.05% Tween 20 at room temperature for 2 h. Binding of C1q was detected with either anti-human C1q:HRP conjugate (A) or monoclonal anti-human C1q antibody combined with HRP-conjugated secondary antibody (B). (C) C1q and sRAGE binding by Dot blot. 1 μl of sRAGE was loaded onto nitrocellulose membrane, detection of C1q binding was performed as described in Section 2. (D) SPR study of C1q and sRAGE binding. sRAGE was used as bait protein; different concentrations of C1q in PBS were used as prey protein. Association and dissociation signals were monitored by surface plasmon resonance. (E) Steady state binding of C1q and sRAGE. 0-800 ng/ml of C1q in PBS containing 2% BSA was incubated in sRAGE-coated or BSA-coated MaxiSorp plate at room temperature for 1 h. C1q levels remaining in solution (free C1q) were determined by C1q ELISA; bound C1q was calculated as the difference of free C1q concentrations between BSA-coated wells and sRAGE-coated wells. Results in A, B and E were mean±SD of three independent experiments. *p<0.01 as compared to BSA. Results in C and D were representatives of three independent experiments. (F) MaxiSorp plate coating and blocking were similarly performed as in FIG. 1. The resulted wells were incubated with 0.1 ml of 2 μg/ml of C1q in 2% BSA/PBS solution with 0.05% Tween 20 at room temperature for 2 hours. Binding of C1q was detected with anti-human C1q:HRP conjugate and o-Phenylenediamine dihydrochloride as final development substrate.

FIG. 11. Pull-down of human sRAGE by immobilized human C1q under different ionic strength conditions.

Pull-down of sRAGE by immobilized BSA and C1q was tested in two different solutions, one was physiological solution—PBS and the other was four-times diluted physiological solution—¼ PBS. Pure human sRAGE protein was used for positive control in Western blot detection of pull-down sRAGE. A representative of three independent experiments was shown.

FIG. 12. RAGE bound to globular head of C1q.

Two monoclonal antibodies for globular head (anti-gC1q) and collagen region of C1q (anti-cC1q) were used to study the RAGE binding site on C1q. (A) 30 min preincubation of 2 μg/ml of C1q with 0-10 μg/ml of anti-gC1q in PBS containing 2% BSA and 0.05% between 20 blocked C1q binding to immobilized sRAGE (10 μg/ml) in an ELISA-like assay. (B) Detection of both C1q binding and anti-gC1q binding to immobilized sRAGE after preincubation of C1q with 5 μg/ml of anti-gC1q performed as in (A). (C) Detection of both C1q binding and anti-cC1q binding to immobilized sRAGE after preincubation of C1q with 5 μg/ml of anti-cC1q performed like in (A). “+” and “−” in (B) and (C) indicate whether the C1q was preincubated with monoclonal antibody or not, respectively. Results in (B) and (C) were mean±SD of three independent experiments. *p<0.01 in pair comparison indicated in the graph.

FIG. 13. Formation of a complex structure of human Mac-1, sRAGE and C1q.

A. Pull-down of sRAGE and C1q by immobilized Mac-1 was performed in a 96-well MaxiSorp plate in 2-times diluted PBS. Western blots were used to detect the pull-down amount of sRAGE and C1q. Pure human sRAGE (5 ng) and C1q (17 ng) proteins were used for positive controls in Western blot detection of pull-down sRAGE and C1q. Result was a representative of three independent experiments.

B. 2 μg of recombinant human Mac-1 (R&D Systems) and 0.4 μg of sRAGE from HEK293 cells were incubated in solution together and separately in 3 μl of PBS at 25° C. for 30 min; control condition was 3 μl of PBS only, then 7 μl of human serum was added to start 1 hour incubation at 37° C. C3a was assayed by ELISA kit.

FIG. 14. Human sRAGE activates complement in a C1q-dependent manner.

C3a levels in fresh human serum (A), C1q-depleted fresh human serum (B) and Mg-EGTA (5 mM Mg Cl₂ and 10 mM EGTA) treated fresh human serum (C) were assayed after 1 h incubation at 37° C. in a 96-well NUNC MaxiSorp plate coated with 20 μg/ml of human sRAGE. (D) Complement activation by a fusion protein rhsRAGE/Fc and its Fc fragment. Two different forms of each protein were tested, one was immobilized and the other was free molecule in serum (dissolved). For immobilized forms, 96-well MaxiSorp plate was coated with 50 μg/ml of each protein and blocked with BSA. Complement activation was performed as in (A). For soluble forms of these two proteins, a mixture of 30 μl of fresh human serum and 10 μl of either rhsRAGE/Fc or Fc (200 μg/ml) was incubated for 1 h at 37° C. in a 5% CO2 incubator. Results in (A), (B) and (C) were mean±SD of four independent experiments each performed in triplicate. Results in (D) were mean±SD of three independent experiments. *p<0.01 as compared to BSA.

FIG. 15. Molecular interaction of RAGE and C1q could induce attraction between RAGE-expressing cells and C1q-opsonized beads or surface.

(A) ARPE-19RAGE cells expressed high level of RAGE protein. Ten micrograms of total proteins from ARPE-19, ARPE-19RAGE and RAGE-transfected C6 cells (C6-RAGE) were analyzed for RAGE expression by Western blot. (B) Recruitment of C1q-coated beads to RAGE-expressing cells. Confluent ARPE-19 and ARPE-19RAGE cells in a 96-well plate were incubated with 0.1 ml of serum-free medium containing 2×108/ml of BSA-coated beads, C1q-coated beads, or C1q-coated beads+20 μg/ml anti-human RAGE (IgY) at 37° C. for 1 h. After washing three-times with HBSS, the plate was read at emission 620 nm with excitation 530 nm. (C) and (D) Adhesion of U937 cells to C1q-coated plate in RAGE and Mac-1 dependent manner. Activated U937 cells were suspended in serum-free RPMI-1640 medium and cultured in a C1q-coated plate for 24 h. After loading of fluorescent calcein and careful washing, U937 cells that were attached to the plate were photographed (C) under both regular light and fluorescent light and quantitatively measured with fluorescence plate reader (D). Involvement of RAGE and Mac-1 molecules in the recruitment of U937 cells were studied with chicken anti-human RAGE IgY and F(ab′)₂ of mouse anti-human CD11b IgG1. As controls, chicken anti-AGEs IgY and mouse IgG1 isotype control were used. Relative fluorescence was calculated as percentage of the fluorescence reading obtained with C1q-opsonized beads at control condition. Results in (B) and (D) were mean±SD of three independent experiments. *p<0.01 in pair comparison indicated in the graph.

FIG. 16. Complement Systems.

FIG. 17. RAGE Activates Complement System through C1.

Human sRAGE was produced and purified from two cell lines: HEK293 and sf9.

FIG. 18. Extracellular Domain of RAGE Can Activate Complement System When C1q-depleted human serum was used.

FIG. 19. (A) Complement Activation by Soluble fusion receptor rhRAGE/Fc (from R & D Systems) (B) Light colored sequences are possible C1q binding sites.

FIG. 20. Expected C1q Binding Site in RAGE has homology with site on IgG (Fc).

FIG. 21. Direct Binding of C1q to Immobilized sRAGE and Avastin (IgG).

FIG. 22. sRAGE Binding to Immobilized C1q.

FIG. 23. Surface plasmon resonance (SPR).

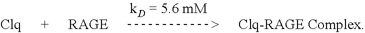

FIG. 24. sRAGE, Mac-1 and C1q form complex structure: (A) complement activation by sRAGE-Mac-1 (B) Pull-down by immobilized Mac-1.

FIG. 25. Myeloperoxidase Activity and Protein Level Decreased in in RAGE −/− Lung Tissue.

FIG. 26. Mac-3 Immunostaining for Macrophages & Neutrophils decreased in RAGE −/−.

FIG. 27. Monocytes/Macrophages Recruitment by C1q.

Both Mac-1 and RAGE are highly expressed in monocytes/macrophages. RAGE-Mac-1 complex is expected to be involved in C1q-enhanced phagocytosis. Chicken anti-RAGE Ab (IgY) reduces C1q-directed recruitment in the results of the studies shown in this figure.

FIG. 28. Evidence that RAGE is involved in effective leukocytotic function.

20 h after intraperitoneal injection of 5×104 colony-forming units (CFUs) of E. coli {Data from The Journal of Infectious Diseases 2009; 200:765-773] wild-type mice were also treated with anti-RAGE IgG or control IgG antibodies and the results were in line with the data obtained in RAGE−/− mice. Total leukocytes, neutrophils, and macrophages in peritoneal lavage fluid (PLF) samples were similar in anti-RAGE- and control antibody-treated mice.

FIG. 29. Schematic of Molecular Interaction Model involving RAGE-C1q.

### DETAILED DESCRIPTION OF THE INVENTION

As used herein, and unless stated otherwise, each of the following terms shall have the definition set forth below.

“Administering to the subject” means the giving of, dispensing of, or application of medicines, drugs, or remedies to a subject to relieve or cure a pathological condition. Oral administration is one way of administering the instant compounds to the subject.

As used herein, “RAGE” is receptor for advanced glycation endproduct. RAGE includes any molecule or peptide having a nucleotide and protein amino acid sequence that is associated with RAGE. RAGE also includes the multiligand signal transduction receptor of the immunoglobulin superfamily of cell surface molecules that mediates a spectrum of distinct cell-specific effects in settings characterized by accumulation of its ligands. RAGE also emcompasses a peptide which has the at least 5 length amino acid sequence which is the same as RAGE.

The nucleotide and protein amino acid sequences that are associated with RAGE are well known, and are described in as shown in Neeper et al. (1992) and U.S. Pat. Nos. 6,563,015; 6,555,651; and 7,258,857 and they are hereby incorporated by reference.

One embodiment of RAGE is a peptide having an amino acid sequence corresponding to the amino acid sequence of a V domain of a RAGE or soluble RAGE is exemplified by the following amino acid sequences:

(SEQ ID No: 1)
A-Q-N-I-T-A-R-I-G-E-P-L-V-L-K-C-K-G-A-P-K-K-P-
P-Q-R-L-E-W-K;
(SEQ ID No: 2)
G-Q-N-I-T-A-R-I-G-E-P-L-V-L-S-C-K-G-A-P-K-K-P-P-
Q-Q-L-E-W-K;
(SEQ ID No: 3)
G-Q-N-I-T-A-R-I-G-E-P-L-M-L-S-C-K-A-A-P-K-K-P-
T-Q-K-L-E-W-K;
(SEQ ID No: 4)
D-Q-N-I-T-A-R-I-G-K-P-L-V-L-N-C-K-G-A-P-K-K-P-
P-Q-Q-L-E-W-K.

The another embodiment provides for an isolated peptide having an amino acid sequence which corresponds to the amino acid sequence of the first 1-112 amino acids of human RAGE (which is the V domain of human RAGE) or which corresponds to amino acids 5-35 of the V domain of human RAGE or any other smaller portion of the V domain human RAGE Representative peptides of the present invention include but are not limited to peptides having an amino acid sequence which corresponds to amino acid numbers (2-30), (5-35), (10-40), (15-45), (20-50), (25-55), (30-60), (30-65), (10-60), (8-100), (14-75), (24-80), (33-75), (45-110) of human sRAGE protein The abbreviations used herein for amino acids are those abbreviations which are conventionally used A Ala Alanine R Arg Arginine N Asn Asparagine D Asp Aspartic acid C Cys Cysteine Q Gln Glutamine E Glu Gutamic acid G Gly Glycine H His Histidine I Ile Isoleucine L Leu Leucine K Lys Lysine M Met Methionine F Phe Phenyalanine P Pro Proline S Ser Serine T Thr Threonine W Trp Tryptophan Y Tyr Tyrosine V Val Valine The amino acids may be L or D amino acids An amino acid may be replaced by a synthetic amino acid which is altered so as to increase the half life of the peptide or to increase the potency of the peptide or to increase the bioavailability of the peptide

As used herein, “Complement C1q” or “C1q” is the first subcomponent of the C1 complex of the classical pathway of complement activation.

As used herein, “Bindng of Receptor for advanced glycation endproduct (RAGE) and C1q” or “RAGE-C1q Binding” is any binding, complexation, bonding or any other attraction or force between RAGE and C1q.

As used herein, the terms “inhibiting,” “inhibit” or “inhibition” of any binding means preventing or reducing the interaction.

The terms “Treat” or “Treating,” a disorder/disease shall mean slowing, stopping or reversing the disorder's progression, and/or ameliorating, lessening, or removing symptoms of the disorder. Thus treating a disorder encompasses reversing the disorder's progression, including up to the point of eliminating the disorder itself.

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention, i.e. a therapeutically effective amount. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, the term “immobilized,” in the context of an immobilized agent, protein, or receptor, refers to a compound that is affixed (e.g., tethered) to a substrate or support (e.g., a solid matrix), and not free in solution.

As used herein, the term “solid matrix” is defined as a solid material of any size, shape, composition or construction that is suitable as an attachment material for any agent, protein, or receptor of the present invention.

The subject invention provides a method of identifying a compound capable of inhibiting binding of receptor for advanced glycation endproduct (RAGE) and Complement C1q (C1q) (RAGE-C1q Binding) Comprising:
a) providing an amount of the compound to be tested;
b) contacting the amount of the compound with
   i) an amount of C1q, and then combining the amount of C1q with an amount of RAGE under conditions that permit RAGE-C1q binding,
   ii) an amount of RAGE, and then combining the amount of RAGE with an amount of C1q under conditions that permit RAGE-C1q binding, or
   iii) a mixture of an amount of C1q and an amount of RAGE under conditions that permit RAGE-C1q binding;
c) measuring the amount of RAGE-C1q binding in step b);
d) comparing the amount of RAGE-C1q binding measured in step c) with the amount of RAGE-C1q binding measured under corresponding conditions in the absence of the compound; and
e) identifying the compound as capable of inhibiting RAGE-C1q binding if the amount of RAGE-C1q binding measured in step c) is less than the amount of RAGE-C1q binding in the absence of the compound under corresponding conditions.

In an embodiment, in step b) C1q is immobilized on a solid matrix.

In an embodiment, in step b) RAGE is immobilized on a solid matrix.

In an embodiment, RAGE is sRAGE.

In an embodiment, RAGE is on the surface of a cell.

In an embodiment, the amount of RAGE-C1q binding is measured by differential centrifugation, chromatography, gel filtration chromatography, ion-exchange chromatography, electrophoresis, immunoprecipitation, pulldown assay, ELISA assay, fluorescence energy transfer, surface plasmon resonance, dot blot, or in vitro tubulin deacetylation assay.

In an embodiment, the compound is tested on a cell.

In an embodiment, the compound is tested in vitro.

In an embodiment, the compound is an antibody, aptamer, a peptide, a small molecule, siRNA or shRNA.

In an embodiment, the subject invention is a compound capable of inhibiting RAGE-C1q binding identified by the subject invention.

The subject invention also provides a method of identifying a compound capable of increasing RAGE-C1q binding comprising:
a) providing an amount of the compound to be tested;
b) contacting the amount of the compound with
   i) an amount of C1q, and then combining the amount of C1q with an amount of RAGE under conditions that permit RAGE-C1q binding,
   ii) an amount of RAGE, and then combining the amount of RAGE with an amount of C1q under conditions that permit RAGE-C1q binding, or
   iii) a mixture of an amount of C1q and an amount of RAGE under conditions that permit RAGE-C1q binding;
c) measuring the amount of RAGE-C1q binding in step b);
d) comparing the amount of RAGE-C1q binding measured in step c) with the amount of RAGE-C1q binding measured under corresponding conditions in the absence of the compound; and
e) identifying the compound as capable of increasing RAGE-C1q binding if the amount of RAGE-C1q binding measured in step c) is more than the amount of RAGE-C1q binding in the absence of the compound under corresponding conditions.

In an embodiment, in step b) C1q is immobilized on a solid matrix.

In an embodiment, in step b) RAGE is immobilized on a solid matrix.

In an embodiment, RAGE is sRAGE.

In an embodiment, RAGE is on the surface of a cell.

In an embodiment, the amount of RAGE-C1q binding is measured by differential centrifugation, chromatography, gel filtration chromatography, ion-exchange chromatography, electrophoresis, immunoprecipitation, pulldown assay, ELISA assay, fluorescence energy transfer, surface plasmon resonance, dot blot, or in vitro tubulin deacetylation assay.

In an embodiment, the compound is tested on a cell.

In an embodiment, the compound is tested in vitro.

In an embodiment, the compound is an antibody, aptamer, a peptide, a small molecule, siRNA or shRNA.

In an embodiment, the subject invention is a compound capable of increasing RAGE-C1q binding identified by the the subject invention.

The subject invention also provides a method of reducing phagocytosis by a phagocyte comprising contacting the phagocyte with an effective amount of a compound capable of inhibiting RAGE-C1q binding, thereby reducing phagocytosis by the phagocyte.

In an embodiment, the compound capable of inhibiting RAGE-C1q binding blocks RAGE's binding sites.

In an embodiment, the compound capable of inhibiting RAGE-C1q binding blocks C1q's binding sites.

In an embodiment, the compound capable of inhibiting RAGE-C1q binding blocks C1q's globular head.

In an embodiment, the compound capable of inhibiting RAGE-C1q binding is a RAGE antigen.

In an embodiment, the compound capable of inhibiting RAGE-C1q binding is an antibody, aptamer, a peptide, a small molecule, siRNA or shRNA.

In an embodiment, the antibody is a RAGE antibody.

In an embodiment, the RAGE antibody is chicken antibody against RAGE, RAGE (H-300) Antibody (N-16) (sc-8230), RAGE (N-16) Antibody (sc-5563), RAGE (9A11) Antibody (sc-80653), RAGE (A-9) Antibody sc-365154, RAGE (A11) Antibody sc-80652, RAGE (D-5) Antibody sc-74535, RAGE (C-20) Antibody sc-8229, RAGE (E-1) Antibody sc-74473, or RAGE (RD9C 2) Antibody sc-33662.

In an embodiment, the compound capable of inhibiting RAGE-C1q binding is sRAGE.

In an embodiment, the sRAGE is Human sRAGE from HEK293.

In an embodiment, the sRAGE is Human sRAGE from Sf9.

The subject invention also provides a method of treating a subject suffering from a disease associated with an increase of RAGE expression comprising administering to the subject a therapeutically effective amount of a compound capable of inhibiting RAGE-C1q binding so as to thereby treat the subject.

In an embodiment, the disease causes inflammation.

In an embodiment, the disease is Age-related macular degeneration (AMD).

In an embodiment, the compound capable of inhibiting RAGE-C1q binding is an antibody, aptamer, a peptide, a small molecule, siRNA or and shRNA.

The subject invention also provides a method of treating a subject suffering from a disease associated with a decrease of RAGE expression comprising administering to the subject a therapeutically effective amount of a compound capable of increasing RAGE-C1q binding so as to thereby treat the subject.

In an embodiment, the compound compound capable of increasing RAGE-C1q binding is an antibody, aptamer, a peptide, a small molecule, siRNA or shRNA.

The subject invention also provides a method of treating a subject in need of treatment of an inflammatory disease, comprising administering to the subject a therapeutically effective amount of a compound capable of inhibiting RAGE-C1q binding on the surface of white blood cells (leukocytes) present in the subject, thereby treating the subject.

The subject invention also provides a method of treating a subject in need of treatment of an inflammatory disease, comprising administering to the subject a therapeutically effective amount of a compound capable of increasing RAGE-C1q binding on the surface of white blood cells (leukocytes) present in the subject, thereby treating the subject.

In an embodiment, the inflammatory disease is symtemic lupus erythematosus (SLE).

The subject invention also provides a method of treating a subject in need of treatment of a disease associated excess apoptosis, comprising administering to the subject a therapeutically effective amount of a compound capable of inhibiting RAGE-C1q binding on the surface of white blood cells (leukocytes) present in the subject, thereby treating the subject.

The subject invention also provides a method of treating a subject in need of treatment of a disease associated with a reduction of apoptosis, comprising administering to the subject a therapeutically effective amount of a compound capable of increasing C1q-RAGE binding on the surface of white blood cells (leukocytes) present in the subject, thereby treating the subject.

In an embodiment, the disease is symtemic lupus erythematosus (SLE)

The subject invention also provides a method of strengthening an immune system response against a bacterial infection and bacterial cells, comprising increasing RAGE-C1q binding between 1) C1q bound to bacterial cells and 2) RAGE, thereby strengthening the immune system response against bacterial infection and bacterial cells.

In an embodiment, the bacterial infection and bacterial cells are Escherichia Coli.

The subject invention also provides a kit for use in identifying a modulator of RAGE-C1q binding comprising:

a) RAGE;

b) C1q; and

c) A candidate Modulator of RAGE-C1q binding.

The subject invention also provides a method of identifying a compound as a modulator of RAGE-C1q binding comprising
a) providing an amount of the compound to be tested;
b) contacting the amount of the compound with
   i.) an amount of C1q, and then combining the amount of C1q with an amount of RAGE under conditions that permit RAGE-C1q binding,
   ii.) an amount of RAGE, and then combining the amount of RAGE with an amount of C1q under conditions that permit RAGE-C1q binding, or
   iii.) a mixture of an amount of C1q and an amount of RAGE under conditions that permit RAGE-C1q binding;
c) measuring the amount of RAGE-C1q binding in step b)
d) comparing the amount of RAGE-C1q binding measured in step c) with the amount of RAGE-C1q binding measured under corresponding conditions in the absence of the compound; and
e) identifying the compound as a modulator of RAGE-C1q binding, if the amount of RAGE-C1q binding measured in step c) is different than the amount of RAGE-C1q binding in the absence of the compound under corresponding conditions.

In an embodiment, in step b) C1q is immobilized on a solid matrix.

In an embodiment, in step b) RAGE is immobilized on a solid matrix.

In an embodiment, RAGE is sRAGE.

In an embodiment, RAGE is on the surface of a cell.

In an embodiment, the invention is a compound identified as a modulator of RAGE-C1q binding by the subject invention.

The subject invention also provides a method of treating a subject suffering from a RAGE-related disorder comprising administering to the subject a therapeutically effective amount of a compound that is a modulator of RAGE-C1q binding so as to thereby treat the subject.

In an embodiment, the RAGE related-disease is chronic inflammation, Alzheimer's disease, diabetes, diabetic complications, an autoimmune disease, allergies, pulmonary vascular diseases, arthritis, Age-related macular degeneration (AMD), macular degeneration, glaucoma, an infection, a general chronic inflammation disease, an inflammation, intense inflammatory response, arthritis, rheumatoid arthritis, systemic lupus erythematosus (SLE), C1q nephropathy, atherosclerosis, obesity, asthma, a vascular disease, chronic obstructive pulmonary disease, cystic fibrosis, abdominal sepsis caused by Escherichia coli, sepsis, angiogenesis, cancer, heart disease, a pulmonary disorder, diabetes associated nephropathy or Multiple sclerosis, transplant rejection, tumor growth, metastasis of a cancer, complications due to diabetes, retinopathy, neuropathy, impotence, impaired wound healing, gastroparesis, Huntington's disease, amyotrophic lateral sclerosis, neointimal formation, amyloid anngiopathy, glomerular injury, seizure-induced neuronal damage, acute skin inflammation, psoriasis, atopic dermatitis, renal failure, hyperlipidemic atherosclerosis associated with diabetes, diabetic late complication increased vascular permeability, diabetic late complication nephropathy, diabetic late complication retinaopathy, diabetic late complication neuropathy, neuronal cytotoxicity, amyothropic lateral sclerosis, multiple sclerosis, dementia associated with head trauma, neuronal degeneration, restenosis, down's syndrome, amyloidosis, periodontal disease, or erectile disfunction.

Based upon the foregoing, the present invention also provides systems and methods for use in screening for, and identifying, modulators of RAGE and C1q interaction. As used herein, a “modulator of RAGE and C1q interaction” may be any agent or combination of agents that that has an antagonistic (inhibitory) or agonistic (facilitatory) effect on RAGE and C1q interaction, including any form of RAGE such as, but not limited to, cellular RAGE and soluble RAGE and any form of C1q. Thus, a modulator of RAGE and C1q interaction may be an agonist or an antagonist. The modulators of the present invention, including any now known or later discovered, also may be natural or synthetic.

Each embodiment disclosed herein is contemplated as being applicable to each of the other disclosed embodiments. Thus, all combinations of the various elements described herein are within the scope of the invention.

All combinations and sub-combinations of each of the various elements of the methods and embodiments described herein are envisaged and are within the scope of the invention.

This invention will be better understood by reference to the Experimental Details which follow, but those skilled in the art will readily appreciate that the specific experiments detailed are only illustrative of the invention as described more fully in the claims which follow thereafter.

This invention will be better understood by reference to the Examples which follow, which are set forth to aid in an understanding of the subject matter but are not intended to, and should not be construed to, limit in any way the claims which follow thereafter.

### EXPERIMENTAL DETAILS

### Example 1

### Rage Binds C1q and Enhances C1q-Mediated Phagocytosis

Using soluble forms of RAGE (sRAGE) and RAGE-expressing cells, the inventors determined that RAGE is a native C1q globular domain receptor. Direct C1q-sRAGE interaction was demonstrated with surface plasmon resonance (SPR), with minimum K_{d} 5.6 μM, and stronger binding affinity seen in ELISA-like experiments involving multivalent binding.

Pull-down experiments suggested formation of a receptor complex of RAGE and Mac-1 to further enhance affinity for C1q. C1q induced U937 cell adhesion and phagocytosis was inhibited by antibodies to RAGE or Mac-1. These data link C1q and RAGE to the recruitment of leukocytes and phagocytosis of C1q-coated material.

### Materials:

Three forms of human sRAGE, the extracellular domain of the receptor, were utilized. One was prepared with a baculovirus expression system using Sf9 insect cells as previously described (Park, 1998). The second one, which was produced as a recombinant protein in human HEK293 cells, was purchased from BioVendor (Candler, N.C.). The third sRAGE molecule, which was a chimeric protein that combines the extracellular domain of human RAGE and the Fc region of human IgG1, was expressed in a mouse myeloma cell line-NSO and purchased from R&D Systems (rhsRAGE/Fc, Minneapolis, Minn.). Normal human serum, C1q-depleted human serum, human C1q protein, human C3a EIA kit were all obtained from Quidel (San Diego, Calif.). Sheep anti-human C1q:HRP conjugate and mouse anti-human C1q (clone 3R9/2) were purchased from AbD Serotec (Raleigh, N.C.). Mouse anti-human C1q (clone JL-1) was purchased from Abcam (Cambridge, Mass.). S100B was obtained from Calbiochem (La Jolla, Calif.). Recombinant human TGF-β1 and recombinant Mac-1 were obtained from R&D Systems. Monoclonal mouse anti-human CD11b (clone ICRF44), 1α,25-dihydroxyvitamin D₃ (vitamin D₃), staurosporine, Calcein-AM, glycated bovine serum albumin (AGE-BSA), amyloid β protein fragment 1-42 (A-beta) and red fluorescent sulfate-modified Latex beads were obtained from Sigma (St. Louis, Mo.). Chloromethylbenzamido derivative of 3,3′-dioctadecylindocarbocyanine (CM-DiI) and carboxyfluorescein diacetate succinimidyl ester (CFDA-SE) were purchased from Invitrogen (Carlsbad, Calif.). F(ab′)₂ fragments of mouse anti-human CD11b were prepared by using the Mouse IgG1 Fab and F(ab′)₂ Preparation kit (Pierce, Rockford, Ill.) according to the manufacturer's protocol. Chicken anti-human RAGE, chicken anti-AGEs and rabbit anti-human RAGE were prepared as previously described (Hori 1995; Reiniger, 2010; Barile, 2005).

### Cell Culture and Transfection:

Human monocyte cell line, U937, human retinal pigment epithelial cell line, ARPE-19, and Jurkat cells were purchased from ATCC (Manassas, Va.). All cells were maintained at 37° C. in a 5% CO₂ incubator. U937 and Jurkat cells were cultured in RPMI-1640 medium, ARPE-19 cells were cultured in Dulbecco's modified Eagle's medium, both media were supplemented with 10% heat-inactivated fetal bovine serum (FES), 100 IU/ml penicillin, and 100 μg/ml streptomycin. U937 and Jurkat cells were maintained at 1×10⁵-3×10⁶ cells/ml with addition of fresh medium every 2-3 days. Subculture of ARPE-19 cells were performed with 0.05% trypsin-EDTA solution, and cell culture medium was changed regularly every 3-4 days. The human RAGE gene subcloned into the pcDNA3 plasmid, pcDNA3 (RAGE) (Taguchi, 2000), was used to transfect ARPE-19 cells by using Lipofectamine 2000 (Invitrogen), according to the manufacturer's protocol. Cells were selected in the presence of geneticin (G418), 1.0 mg/ml, and individual clones were isolated by limiting dilution. After analysis of RAGE expression in these clones, RAGE over-expressing cell line was generated as ARPE-19RAGE.

### Immunofluorescence Microscopy:

U937 cells (2×10⁵/ml) were differentiated in standard medium containing 100 nM of vitamin D₃ and 2 ng/ml of TGF-β1 at 37° C. in a 5% CO₂ incubator for 3 days, with vitamin D₃ and TGF-β1 refreshments everyday (Defacque, 1999; Gingras, 2000). After fixation of cells with 4% paraformaldehyde, immunofluorescence staining was performed with 1:50 dilution of chicken anti-human RAGE IgY and 1:100 dilution of mouse anti-human CD11b monoclonal IgG1. Goat secondary antibodies labeled with Alexa Fluor 546 and 488 were used for microscopy of RAGE and Mac-1, respectively.

### Flow Cytometry and Analysis of Phagocytosis:

U937 cells (2×10⁵/ml) were differentiated in standard medium containing 100 nM of vitamin D₃ and 2 ng/ml of TGF-β1 at 37° C. in a 5% CO₂ incubator for 2 days, with refreshment of vitamin D₃ and TGF-β1 at 24 h. Differentiated U937 cells were harvested and re-suspended in Hank's Balanced Salt Solution (HESS, containing calcium and magnesium) at 2×10⁶/ml, and then labeled with 5 μM of CM-DiI at 37° C. for 20 min. Labeling was ended by washing cells three times with HESS. The resulted cells were re-suspended at 4×10⁵/ml in phagocytosis buffer (HESS plus 2 mM MgCl₂ and 20 mM glutamate, pH 7.0).

To label Jurkat cells with CFSE (carboxyfluorescein succinimidyl ester), Jurkat cells were incubated with 1 μM of CFDA-SE in HESS for 30 min at 37° C. Harvested cells were resuspended (1×10⁶/ml) in HESS containing 1 μM of staurosporine and incubated at 37° C. for 6 h. Apoptotic cells were confirmed to reach 75-85% of total Jurkat cells routinely by annexin V and propidium iodide staining. Apoptotic Jurkat cells were washed with HESS and re-suspended at 1.6×10⁶/ml in phagocytosis buffer.

Mixtures of U937 cells and apoptotic Jurkat cells prepared as above were incubated in the presence of 0-200 μg/ml of purified human C1q for 2 h at 37° C. or 4° C. Analysis of phagocytosis was performed by using a FACS Calibur flow cytometer and the Cell Quest Pro software (BD Biosciences, San Diego, Calif.). Enhancement of phagocytosis by C1q opsonization reached maximum at 20 μg/ml of C1q, thus subsequent phagocytosis studies with blocking antibodies for phagocytic receptors were performed with this C1q concentration. To minimize binding of antibody to Fc receptors and complement component proteins, chicken antibodies and F(ab′)₂ of mouse monoclonal antibody were used in these experiments. Chicken anti-human RAGE IgY and its control antibody, chicken anti-AGEs IgY, were both used at μg/ml, F(ab′)₂ of mouse monoclonal anti-human CD11b and its control mouse IgG1 isotype were used at 20 μg/ml and 100 μg/ml, respectively.

### Phagocytosis of Latex Beads:

Ten microliters of red fluorescent sulfate-modified Latex beads (0.5 μm, 1×10¹¹ beads/ml) were opsonized with 10 μl of 1 mg/ml of C1q in PBS at 4° C. overnight. Remaining binding sites were blocked with 10% heat-inactivated FBS in PBS at 25° C. for 3 h. Beads were suspended at 5×10⁹ beads/ml in PBS. U937 cells (2×10⁵/ml) were activated in standard medium containing 100 nM of vitamin D₃ and 2 ng/ml of TGF-β1 at 37° C. in a 5% CO₂ incubator for 24 h. After washing twice with serum-free RPMI-1640 medium, U937 cells were suspended at a concentration of 2×10⁵/ml in serum-free RPMI-1640 medium containing 2×10⁸/ml of FOS-coated beads or C1q-coated beads. Chicken anti-human RAGE IgY or F(ab′)₂ of mouse anti-human CD11b IgG1 were used as described in monocyte adhesion experiments to block ligation of RAGE and Mac-1, respectively. Control antibodies were also tested. The suspensions of cells and beads were then incubated in a 5% CO₂ incubator with occasional shaking at 37° C. for 4 h. Free beads were washed by pelleting and re-suspending the cells three times with cold Hanks' balanced salt solution. The phagocytosed beads were quantified by fluorescence readings with excitation at 530 nm and emission at 620 nm, and a small variation of cell number was corrected by readings at 280 nm.

### Protein Binding Assays:

Three different techniques were used to detect protein-protein interaction of RAGE and C1q: (1) ELISA-like assays; (2) pull-down; (3) surface plasmon resonance (SPR).

### ELISA-Like Assays:

96-well NUNC MaxiSorp plates were coated with 100 μl of 0-50 μg/ml of sRAGE in PBS at 4° C. overnight; remaining binding sites were blocked with 2% BSA in PBS for 2 h at room temperature. The wells were incubated with 0.1 ml of 2 μg/ml of C1q in PBS containing 2% BSA and 0.05% Tween 20 at room temperature for 2 h. Stably bound C1q was then specifically recognized by anti-human C1q:HRP conjugate in a ELISA-like procedure. The final peroxidase activity was monitored at 492 nm with o-phenylenediamine dihydrochloride and hydrogen peroxide as substrates. A distinct dot blot-like assay was performed on the surface of a nitrocellulose membrane as follows: 1 μl of sRAGE in PBS was spotted onto a dry nitrocellulose membrane; after the membrane was air-dried, it was washed thoroughly with PBS. Remaining binding sites were blocked with 5% non-fat milk in PBS. C1q binding was carried out with 5 μg/ml of C1q in 5% non-fat milk at room temperature for 2 h and detected with anti-human C1q:HRP conjugate. ECL Western blot detection kit was used to visualize the binding.

### Pull-Down:

96-well NUNC MaxiSorp plate was coated with 0.1 ml of 0.1 mg/ml of human C1q in PBS at 4° C. overnight. After blocking with 2% BSA in PBS, the plate was incubated with 0.1 ml sRAGE (0.1 mg/ml) in 2% BSA in PBS at room temperature for 1 h. After the wells were washed quickly three times with cold PBS, NuPAGE sample buffer was used to denature and collect sRAGE from the plate for Western blot detection. To study the effect of ionic strength on sRAGE binding to immobilized C1q, PBS used in the above binding procedure was substituted with ¼ dilution of PBS, and the pull-down of sRAGE was analyzed in the same manner as above. To study the possibility of complex formation of sRAGE, C1q, and Mac-1, 96-well NUNC MaxiSorp plate was coated with 50 μl of 25 μg/ml of Mac-1 in ½ diluted PBS (½ PBS) at 4° C. overnight, then blocked with 5% BSA in ½ PBS. After washing three-times with ½ PBS, 50 μl of sRAGE (10 μg/ml, from HEK293 cells) or C1q (100 μg/ml) or both proteins in ½ PBS containing 2% BSA was then added to each well and incubated at room temperature for 1 h. The plate was cooled down on ice for 10 min and washed quickly three-times with cold ½ PBS. SDS-NuPAGE sample buffer was used to dissolve proteins in the plate and used for electrophoresis and Western blots of C1q and sRAGE. Stripping of Western blot membranes was performed with buffer from Pierce according to the manufacturer's instructions. Antibodies used in Western blot were rabbit anti-human RAGE (2 μg/ml) and sheep anti-human C1q:HRP conjugate (1:500).

### Surface Plasmon Resonance (SPR):

The SPR analysis was carried out with the Biacore X system (GE Healthcare, Pittsburgh, Pa.). Sensor chips, amine coupling kit, immobilization and running buffers, and regeneration solutions were obtained from GE Healthcare. The carboxyl groups on the CM5 sensor chips were activated for 7 min using 0.1 M N-hydroxysuccinimide (NHS) and 0.4 M (N-ethyl-N′-(3-dimethylaminopropyl) carbodiimide (EDC) mixed at 1:1 (v/v) ratio. In the coupling step, a 30 μl injection of 0.15 mg/ml sRAGE (in sodium acetate buffer at pH 5.5) was flowed over the activated surface for 6 min. The remaining activated sites on the chip surfaces were blocked with a 35 μl injection of an ethanolamine hydrochloride solution (1 M at pH 8.5), followed by a 60 s wash with 2 M NaCl to remove any nonspecifically adsorbed materials. About 3800RU of the immobilized sRAGE proteins were obtained.

C1q dilutions were prepared in PBS buffer and injected sequentially over two flow cells at a flow rate of 10 μl/min. Surface regeneration was achieved using a 20 s injection of 10 mM glycine-HCl, pH 2.0 as regeneration buffer. The response curves were obtained by subtraction of the signals over the reference surface from the binding response to RAGE immobilized surface. Evaluation was carried out using BIAevaluation Software (GE Healthcare). The response at steady-state was obtained by fit of sensorgrams to standard binding models, where appropriate, or calculated as responses at late association or early dissociation phase using the steady state affinity function in BIAevaluation. Affinity was determined from kinetic analysis (on- and off-rates) or as the apparent affinity after plotting responses versus concentration of analyte in a saturation curve.

### Complement Activation Assays:

96-well NUNC MaxiSorp plate was coated with 100 μl of 20 μg/ml of sRAGE in PBS at 4° C. overnight; remaining binding sites were blocked with 2% BSA in PBS for 2 h at room temperature. Complement activation was initiated with incubation of normal human serum (20 μl) or C1q-depleted human serum in the plate at 37° C. in a 5% CO₂ incubator. After 1 h incubation, C3a levels in the human serum were determined as a marker of complement activation with human C3a EIA kit according to the manufacturer's instructions.

### Monocyte Adhesion:

U937 cells (2×10⁵/ml) were incubated in standard medium containing 100 nM of vitamin D₃ and 2 ng/ml of TGF-β1 at 37° C. in a 5% CO₂ incubator for 24 h. After washing twice with serum-free RPMI-1640 medium, U937 cells were suspended at concentration of 2×10⁵/ml in serum-free RPMI-1640 medium. 100 μg/ml of chicken anti-human RAGE IgY or 20 μg/ml of F(ab′)₂ of mouse anti-human CD11b IgG1 were used in the medium to block RAGE and Mac-1, respectively. As controls, 100 μg/ml of chicken anti-AGEs IgY and 100 μg/ml of mouse IgG1 isotype control (from R&D Systems) were used. 0.1 ml aliquots of cell suspensions were added into 96-well MaxiSorp plate, which had been coated with 30 μg/ml of human C1q and blocked with 10% of heat-inactivated FBS. The cells were cultured at 37° C. in a 5% CO₂ incubator for 24 h, then 50 al of serum-free RPMI-1640 medium containing 15 μM Calcein AM was added into each well, and the plate was incubated at 37° C. in a 5% CO₂ incubator for 1 h to label U937 cells with fluorescence. After three gentle washes with Hanks' balanced salt solution, the stably attached U937 cells were photographed with regular light and fluorescent light. The plate was also read with fluorescence plate reader with 485 nm of excitation and 530 nm of emission.

### Results of Example 1:

### RAGE and Mac-1 in C1q Enhanced Phagocytosis:

Monocytes and monocyte-derived cells such as dendritic cells and macrophages express both RAGE and Mac-1 (CD11b/CD18) on their plasma membrane. Human monocyte cell line U937 is of myeloid lineage, and treatment with Vitamin D₃ and TGF-β1 is widely used for U937 cell differentiation into mature phagocytes. As shown in FIG. 8A, the constitutive expression of RAGE and CD11b in undifferentiated U937 cells is very low; with vitamin D3 and TGF-β1 stimulation, both proteins increased expression over 24-72 h. Immunofluorescence staining of both proteins after 3 days' stimulation is seen in FIG. 8B.

The effect of C1q upon phagocytosis of apoptotic cells was investigated. As expected, human monocyte cells, U937, activated by vitamin D₃ and TGF-β1 for 2 days, effectively phagocytose staurosporine-induced apoptotic Jurkat cells. The basal level of phagocytic U937 cells was 47% of total U937 cells; the addition of C1q significantly enhanced the phagocytic U937 cells population to 66% (FIG. 9A). To study receptors involved in C1q-enhanced phagocytosis, chicken antibody against human RAGE and F(ab′)₂fragments of mouse monoclonal antibody against human CD11b were utilized. Both antibodies substantially attenuated C1q enhancement of phagocytosis (FIG. 9B). While RAGE blockade had no effect on basal phagocytosis, Mac-1 (CD11b/CD18) appeared to have roles in both basal phagocytosis and C1q enhancement given the results observed with CD11b antibody.

As the clearance of apoptotic cells is a complex process given the presence of many surface molecular and structural changes and subsequent interactions with numerous phagocytic receptors, the study of complement enhancement on phagocytosis and involvement of surface molecules may be hampered in such systems. A more simplified and more specific system for study of C1q enhancement on phagocytosis was then utilized. In this system, latex beads coated with FES or C1q were used in place of apoptotic cells. FES-coated beads did not undergo phagocytosis, differing from the basal phagocytosis seen with apoptotic Jurkat cells. In contrast, C1q-coated beads underwent phagocytosis by differentiated U937 cells, and RAGE and CD11b antibodies significantly attenuated this process (FIGS. 9C and 9D). Antibody blockade showed more dramatic effects in this more specific phagocytosis model. These data demonstrated the involvement of RAGE and Mac-1 in C1q-enhanced phagocytosis. Of note, trypsin treatment of phagocytic U937 cells did not release C1q beads from these cells, confirming that the fluorescence signals were due to phagocytosis of C1q beads.

### C1q-sRAGE Interaction Studies:

The core C1q binding site on human IgG1 molecules is ₃₁₈ExKxK₃₂₂, located at CH2 domain of the Fc portion of the molecule; replacement of amino acid E in this core binding site with V reduces its binding affinity by 90% (Duncan, 1988). Protein sequence analysis reveals a possible C1q-binding site on RAGE's N-terminal residues 35-39, VLKCK (note that residues 1-22 are the signal peptide for RAGE), The Table 1 below shows the highly matched regions surrounding this core C1q binding site on human IgG1H and RAGE from different species,

TABLE 1
Consensus sequences of C1q binding site in RAGE and human IgG1H.

| | |
|---|---|
| Human IgG1H | 294 EQYNSTYRVVSVLTVLHQDWLNGKEYKCKVSNKALPAPIEKTISKAKGQP 343 (SEQ ID No: 5) |
| Human RAGE | 23 AQNITARIGEPLVLKCKGAPKKPPQRLEWKLNTGRTEA 60 (SEQ ID No: 6) |
| Chimpanzee RAGE | 23 AQNITARIGEPLVLKCKGAPKKPPQRLEWKLNTGRTEA 60 (SEQ ID No: 7) |
| Rhesus monkey | 23 AQNITARIGEPLVLKCKGAPKKPPQQLEWKLNTGRTEA 60 (SEQ ID No: 8) |
| RAGE | |
| House mouse RAGE | 23 GQNITARIGEPLVLSCKGAPKKPPQQLEWKLNTGRTEA 60 (SEQ ID No: 9) |
| Norway rat RAGE | 23 GQNITARIGEPLMLSCKGAPKKPTQKLEWKLNTGRTEA 60 (SEQ ID No: 10) |
| Cattle RAGE | 23 DQNITARIGKPLVLNCKGAPKKPPQQLEWKLNTGRTEA 60 (SEQ ID No: 11) |
| Pig RAGE | 23 DQNITARIGKPLVLNCKGAPKKPPQQLEWKLNTGRTEA 60 (SEQ ID No: 12) |
| Dog RAGE | 25 DRNITARIGKPLVLNCRGAPKKPPQQLEWKLNTGRTEA 62 (SEQ ID No: 13) |
| Gray short-tailed | 21 SQNISSRIGEPLVLNCKGAPRKPPQQLEWKLNTRRTEA 58 (SEQ ID No: 14) |
| opossum RAGE | |

In addition to the matched core C1q binding site, other residues reported to be important for C1q binding are also matched, such as 321C, 326K, 329P, 333E in IgG1H and 38C, 43K, 46P, 50E in RAGE (Gaboriaud, 2003; Idusogie, 2000; Kaul, 1997). It is also important to note that both sequences in this region have crowded, charged residues and that most of them match perfectly, suggesting that electrostatic attraction likely plays an important role in C1q-sRAGE interaction as it does in C1q-IgG interaction. In addition, both IgG1H and RAGE have N-linked oligosaccharides located near their core C1q binding site, e.g. 297 N for IgG1H and 25 N for RAGE (Duncan, 1988; Gouras 2008; Kaul, 1997; Neeper, 1992.) that probably contribute to the recognition and binding of C1q.

Direct evidence of C1q-sRAGE interaction was demonstrated with ELISA-like and surface plasmon resonance experiments. Immobilized sRAGE on a cluster plate readily pulls down C1q (purchased from Quidel, catalog number: A400) from aqueous solution and can withstand many steps of washing and incubation in an ELISA-like procedure. Detection of the C1q that remains attached to immobilized sRAGE with an anti-C1q antibody conjugated with HRP showed comparable binding for C1q between the two human forms of sRAGE (FIG. 10A). To assess the possible involvement of AGEs in the interactions, C1q proteins were tested for the presence of AGEs with an ELISA assay (Barile, 2005). These results were negative (data not shown), indicating the binding of C1q and sRAGE was not related to the presence of AGEs. When solution of purified C1q was replaced with native human serum, immobilized sRAGE could also pull-down C1q, effectively, even after 500-fold dilution of the serum (FIG. 10B). FIG. 10F shows a comparison between human sRAGE and human IgG1 (Avastin) upon C1q binding with varying amounts of both proteins for coating. To further confirm the interaction of C1q and sRAGE, dot blot was also utilized and the result shown in FIG. 10C. Of note, when denatured and reduced sRAGE were tested in a Western blot-like procedure, C1q binding abilities were lost (data not shown), indicating the native conformation of sRAGE is required for its binding with C1q. Association and dissociation kinetics from SPR recording, as shown in FIG. 10D, display multiphasic binding curves indicating heterogeneous binding between immobilized sRAGE and liquid phase C1q. This data fits well with the structure of C1q which has six globular heads for binding, the weakest binding is formed through only one C1q head and the strongest binding involves all six heads. Weak binding gave an initial K_{d} value of 5.6 μM, a possible value for one to one molecular binding of sRAGE and C1q, and stronger binding could last long enough to withstand the ELISA-like procedure mentioned above, consistent with multivalent binding of C1q to more than one immobilized sRAGE molecule. Steady-state binding of C1q was also studied with immobilized sRAGE on MaxiSorp plate. Concentrations of C1q remained in solution at steady state equilibrium were assayed, and C1q that bound to sRAGE in sRAGE-coated wells was calculated, as the change of C1q concentration from its control condition (BSA-coated wells). The C1q binding kinetics is shown in FIG. 10E, with the overall K_{d} value at approximately 0.3 nM. The known RAGE ligands 51003, A-beta, and AGE-BSA are unable to block C1q binding to immobilized sRAGE (data not shown). These studies suggest that the RAGE binding site for C1q is distinct from other ligands and/or that multivalent binding of C1q to immobilized sRAGE is too strong to be disrupted by other soluble ligands.

When C1q was coated on a cluster plate, it could also pull-down sRAGE in aqueous solution (FIG. 11 lanes 3 and 5). As C1q-IgG interaction is ionic strength dependent, we examined the effect of ionic strength upon C1q-sRAGE binding using PES solution diluted four-times as a medium for binding studies. Under these conditions, as shown in FIG. 11 lane 5, C1q-sRAGE binding was greatly enhanced. C1q-sRAGE binding was enhanced to such a high level that less than 5 seconds' exposure generated an overexposed autoradiograph. The higher molecular weight protein band in FIG. 11 lane 5 is sRAGE dimer with molecular weight about 90 kDa. In comparison to original sRAGE FIG. 11 (lane 6), this pull-down process also identifies sRAGE oligomers that are known to exist in sRAGE preparations (Xie, 2007).

RAGE Interacts with Globular Head of C1q:

Sequence analysis indicates the C1q binding site on RAGE is similar to IgG, which binds to globular domain of C1q; thus it is conceivable that RAGE also binds to the globular head of C1q. To study the RAGE binding domain on C1q, two monoclonal antibodies were incubated with C1q prior to the ELTSA-like C1q-RAGE binding assays, one specific for globular head of C1q (clone 3R9/2) and the other specific for the collagen domain of C1q (clone JL-1). While antibody to the globular head blocked C1q binding to immobilized sRAGE efficiently and completely (FIGS. 12A and 12B), antibody to the collagen domain had no effect on the C1q-sRAGE binding (FIG. 12C). The coexistence of these antibodies in the C1q-sRAGE complex was also determined to exclude other unexpected possibilities. Globular head antibody could be detected as low as 0.1 ng in this assay (data not shown), but the inventors did not detect any existence of this antibody in sRAGE-coated wells (FIG. 122), indicating complete blockade of C1q binding to sRAGE by this antibody. In contrast, collagen domain antibody showed co-existence in C1q-sRAGE complex (FIG. 12C). These results further confirmed the observation that RAGE bound to globular head of C1q.

Interaction of sRAGE, C1q, and Mac-1:

Mac-1 is a β2 integrin, and several studies indicate that it may have binding affinity for the collagen domain of C1q (Lauvrak, 1997; Goodman, 1995). It was tested to see if RAGE and its ligand Mac-1 may generate a complex structure that enhances binding for C1q, presuming that RAGE and Mac-1 do not disrupt their respective C1q binding sites in such a structure. To test this concept, a recombinant form of human Mac-1 (R&D Systems, Minneapolis, Minn., catalog number: 4047-AM) reported to have active conformation for ligand binding was employed in a pull-down experiment using immobilized Mac-1 as bait. As shown in FIG. 13, Mac-1 pulled down sRAGE and C1q when they were incubated separately, and Mac-1 pull-down of sRAGE and C1q proteins increased when they were co-incubated. This experiment demonstrates direct interaction of C1q and Mac-1; further testing of their interaction using ELISA-like procedures confirms that immobilized Mac-1 binds C1q (data not shown).

Human sRAGE and Complement Activation:

The primary sequence of RAGE indicates that the RAGE protein resembles immunoglobulin structures (Neeper, 1992). Pure sRAGE molecules were coated on a cluster plate to mimic the immune complex structure of antibody-antigen reactions and complement activation within fresh human serum assayed. To avoid complement activation that would occur due to incompatibility between human serum and RAGE protein, we employed two forms of sRAGE that were both produced by the human RAGE gene, one expressed in insect cells (Sf9) and the other expressed in human kidney cells (HEK 293). Both forms of sRAGE activated the complement system with comparable degrees of activity (FIG. 14A). In contrast, when BSA, HSA (human serum albumin) and heat-inactivated human serum were coated onto the plates, similar basal complement activation (15 μg/ml) was observed, which was subsequently subtracted from experimental data. To characterize the complement pathway that was activated by sRAGE, the experiment was repeated with C1q-depleted human serum. As shown in FIG. 14B, both forms of sRAGE lost their ability to activate the complement system, suggesting that sRAGE interacts with C1 and activates the classical pathway of complement system under these conditions. To further confirm the pathway of complement activation, Mg-EGTA, which blocks both the classical and the mannose binding lectin pathway, was added into native human serum and its effect on sRAGE induced complement activation was studied (FIG. 14C). Like C1q-depleted serum, the addition of Mg-EGTA blocked sRAGE-induced complement activation.

RAGE fusion protein that combines the extracellular domain of human RAGE and the Fc region of human IgG1 via a peptide linker was also tested. This recombinant mature human RAGE/Fc chimera (rhsRAGE/Fc) (rhsRAGE/Fc, available from R&D Systems, Minneapolis, Minn., catalog number: 1145-RG) is a disulfide-linked homodimeric protein that has at least three potential C1q-binding sites, two on RAGE extracellular domains and one on the Fc fragment of IgG1. When compared with the homodimeric recombinant mature human IgG1 Fc that also has peptide linker attached, this fusion protein displayed distinct activity upon complement activation (FIG. 14D). Immobilization of both proteins activated the classical complement pathway. In contrast to the Fc dimer, rhsRAGE/Fc dimer in solution also activated complement. These data suggest that multivalent binding of RAGE molecules with C1q is necessary to alter C1q conformation to lead to activation of C1r which in turn activates C1s.

C1q-RAGE interaction studies at cellular level:

To study C1q binding to RAGE expressing cells, the inventors generated a stable RAGE over-expressing cell line—ARPE-19RAGE from the human retinal pigment epithelial cell line ARPE-19. RAGE expression was compared with another RAGE over-expressing cell line C6-RAGE, generated from rat astroglial C6 (Taguchi, 2000), with the result shown in FIG. 15A. Similar to studies of low affinity Fcγ receptors in which the low affinity for monomeric IgG averts potential nonspecific activation of proinflammatory responses (Nimmerjahn, 2008), free C1q did not bind to RAGE-overexpressing cells when steady state binding method depicted in

FIG. 10E was used (data not shown). Consistent with these C1q binding results, complement activation studies with native human serum incubation of these cells showed no complement activation (data not shown). The results using rat native serum with RAGE-transfected C6 cells produced similarly unremarkable results (results not shown).

Next the inventors tested the interaction of C1q-coated Latex beads with RAGE-overexpressing cells. While ESA-coated beads showed no attachment to either ARPE-19 or ARPE-19RAGE cells, C1q-coated beads demonstrated recruitment of both cell lines, significantly more for ARPE-19RAGE cells, an effect that could be attenuated by RAGE antibody (FIG. 15B). RAGE antibody treatment had no significant effect on the recruitment of C1q-coated beads to ARPE-19 cells. Trypsin treatment could release all attached C1a beads from both cell lines, indicating no phagocytosis activity of these cells for C1q-opsonized beads.

To further confirm the interaction of cellular RAGE with immobilized C1q, an adhesion assay of RAGE-expressing U937 cells to C1q-coated cluster plates was performed. Differentiated U937 cells had very weak adhesion to FBS-coated plate and could be readily washed off by washing steps in the assay, but the cells attached firmly to the C1q-coated plate (FIG. 15C). RAGE antibody and Mac-1 antibody significantly attenuated the recruitment of U937 cells to C1q-coated plate (FIG. 15D).

### Discussion of Example 1:

This study provides the first evidence that RAGE is a C1q receptor that enhances C1q mediated phagocytosis. RAGE's binding affinity for C1q is comparable to the FcγRIII receptors, both of which are in the μM range. The phagocytic activity of monocytes upon particles and apoptotic cells tasted by C1q is significantly blocked by antibodies to either RAGE or Mac-1, implicating the formation of RAGE and its counterligand/coreceptor Mac-1 in a complex in this process. The identification of RAGE and Mac-1 as a receptor complex has been previously demonstrated in a monocyte cell line THP-1; in those studies, HMGE1 stimulation induced strong colocalization of RAGE with Mac-1 at the leading edge of spreading THP-1 cells (Orlova, 2007).

RAGE and C1q play roles in adaptive immunity, and the identification of RAGE as a C1q receptor suggests an important interaction in such mechanisms. The contrasting roles of C1q-mediated complement activation upon apoptotic cells, as compared to microorganisms as targets of complement, increasingly suggests an immunotolerant role of C1q-opsonized apoptotic cells. RAGE and its counter ligand Mac-1 appear to play important roles in phagocytosis and clearance of C1q-tagged material such as autoantigens in the form of apoptotic cells.

Evidence of RAGE involvement in apoptotic cell removal has been demonstrated in recent studies (Friggeri, 2011; He, 2011; Baneriee, 2010) with peritoneal and alveolar macrophages from RAGE null mice displaying significantly decreased phagocytic activity, as measured by the ingestion of apoptotic neutrophils, compared to wild-type control mice.

Circulating C1q molecules did not bind statically to RAGE-expressing cells in our experiments; therefore, local complement activation did not occur on the surface of RAGE-expressing cells. C1q is mainly biosynthesized and secreted in phagocytes including macrophages, dendritic cells, and microglia; these cells can also synthesize other early complement components of both the classical and lectin pathways to produce sufficient local tissue complement for opsonization without recruitment, of plasma complement. (Nayak, 2010; Lu, 2007) RAGE expression in these cells can provide immediate responses for a C1q opsonizad target. The high level of C1q concentration in peripheral tissue with resident macrophages presumably enhances the clearance of apoptotic cells in an efficient biological process. Eased upon our data, we might expect that cellular RAGE, alone or in complex with Mac-1, aids in phagocytic affinity for C1q tagged to material labeled for phagocytosis while limiting local complement activation and cytotoxicity.

RAGE has a role in the innate immune response to abdominal sepsis caused by Escherichia coli (van Zoelen, 2009). When RAGE null and wild type mice were injected with E. coli into the peritoneal cavity, the mice deficient in RAGE demonstrated an enhanced bacterial outgrowth in their peritoneal lavage fluid (PLF) and distant organs such as liver and lung by 20 h post-injection. However, total leukocytes, neutrophils, and macrophages in PLF were similar in wild type and RAGE mice at the same time point. A weaker defense against the growth and/or dissemination of E. coli was also observed when wild type mice were treated with anti-RAGE IgG or sRAGE (van Zoelen, 2010). The molecular mechanism(s) of RAGE's involvement in antibacterial defense during E. coli sepsis were unknown at that time, but the identification of RAGE as a C1q receptor that aids phagocytosis helps explain the observed leukocyte dysfunction of RAGE null mice in clearing C1q tagged pathogens such as E. coli.

C1q is capable of engaging a broad range of ligands via its heterotrimeric globular domain, including apoptotic cells, CRP, IgG, IgM, and RAGE, with the binding sites located on different subunits for some of these ligands (Kishore, 2004). Interaction between these ligands' binding with C1q has not been well studied, and it is possible that the C1q globular domain attached to apoptotic cells could also bind to its receptors to process clearance. On the other hand, C1q has six globular domains, enough for multiple types of binding. Many complement components have been recently screened in searching for new ligands for RAGE (Ruan, 2010). Among them, C3a bound to RAGE firmly and could be further enhanced by human stimulatory unmethvlated cytosine-guanine-rich DNA A. In that study, a fusion protein of RAGE and Fe was used for ligand binding and compared to Fc fragment of IgG to identify complement ligands. As C1q classically binds to the Cc domain to initiate complement activation, the ability of RAGE to preferentially bind C1q is not possible in such an experiment, precluding the authors of that study from identifying C1q in the list of potential ligands. (Ruan, 2010)

In summary, the data presented here identify RAGE as a native C1a receptor that aids the phagocytic roles of C1q. While RAGE can weakly activate the complement system upon binding C1q, the enhanced phagocytic clearance of C1q-tagged material such as bacteria or apoptotic cellular debris appears to be the major mechanism of RAGE's role as a C1q receptor. Our data also suggest that Mac-1-RAGE complex may be formed to further enhance such phagocytic function. The identification of RAGE as a C1q receptor unveils novel concepts regarding these protein's respective roles in the immune system.

### Example 2

### Binding Studies of RAGE-C1q Interaction

RAGE Expression

Numerous cells constitutively express RAGE on their cellular surface, including but not limited to vascular endothelial and smooth muscle cells, T-lymphocytes, neuronal cells, podocytes and pneumocytes. RAGE is also constitutively present at low levels on the membranes of numerous cells, including vascular endothelial cells, vascular smooth muscle cells, monocytes/macrophages, T-lymphocytes, neuronal cells, podocytes and pneumocytes. (Ramasamy, 2005) Specific to the eye, RAGE is present within corneal endothelial cells, several cells of the neural retina but particularly Müller cells, vascular endothelial cells, and RPE cells. (Barile, 2005; Howes, 2004; Kaji, 2003, Kaji, 2007, Soulis, 1997). Under pathological states including retinal diseases, upregulation of the RAGE gene occurs with accumulation of its ligands, amplifying the receptor-ligand axis. (Barile, 2007, Ramasamy, 2008)

RAGE is also expressed on monocytes, macrophages and dendritic cells (Ballinger, 2005; Ferhani, 2010; Zeng, 2009) and its activation contributes to adaptive immune responses (Manfredi, 2008; Yang, 2007; Chen, 2008; Moser, 2007) Specifically, HMGB1-dependent dendritic cell mobilization, migration and activation are RAGE-mediated in a nonredundant manner (Manfredi, 2008; Yang, 2007), and HMGB1's role as an immune alarmin to activate leukocytes during sepsis can be ameliorated by RAGE blockade (Kokkola, 2005; Liliensiek, 2004; Yang, 2004). RAGE also has a role in adaptive immune responses to auto- and alloantigens, with reduced T cell activation to these antigens in the absence of RAGE and preferential expression of RAGE in Th1 cells.

RAGE is highly expressed at the mRNA and protein levels in early developmental stages. Under normal physiological conditions, RAGE expression occurs in most tissues at relatively low levels, except in the lung where it has the highest constitutive expression. In the developed lung RAGE retains a high basal expression.

Soluble isoforms of RAGE that contain the extracellular domains of the cell surface receptor are also present in the circulation and other organ systems including the vitreous cavity of the eye. (Pachydaki, 2006) Soluble RAGE (sRAGE) with the extracellular ligand-binding domain of RAGE has been generated in a baculovirus expression system to serve as an experimental decoy for RAGE's ligands and to study the effects of RAGE antagonism. (Park, 1995) Studies using sRAGE and genetically modified RAGE knockout mice demonstrate that RAGE blockade suppresses acute and chronic inflammatory stresses. Several studies suggest the possible contributions of RAGE to host defense mechanisms in massive injury, sepsis and infections, with RAGE driving an exaggerated inflammatory response in these conditions. In some infections, there appears to be an essential role for RAGE in bacterial defense. When abdominal sepsis was induced in mice via direct intraperitoneal injection of Escherichia coli (E. coli), RAGE deletion or anti-RAGE IgG resulted in increased bacterial outgrowth in the peritoneal cavity and systemically, in parallel with increased tissue damage, cytokine release and activation of coagulation. (van Zoelen, 2009)

RAGE also binds non-AGE ligands implicated in other disorders, including neurodegenerative diseases (amyloid-β peptide) and acute and chronic inflammatory conditions (S100/calgranulins, a family of proinflammatory cytokines; high mobility group box 1 [HMGB1] or amphoterin; and the leukocyte integrin Mac-1, also known as complement receptor 3 [CR3]).

C1q and C1q receptors

C1q and mannose-binding lectin (MEL), as well as surfactant protein A (SPA), SPD, and ficolin, are members of a unique family of proteins termed defense collagens. This family of macromolecules is characterized by a conserved, collagen-like region of repeating Gly-X-Y triplets contiguous with a noncollagen-like globular domain involved in binding to diverse targets. Specifically for C1q, there are 18 peptide chains in 3 subunits A, B and C. Each subunit has 6 peptides that consist of a Y-shaped pair of triple peptide helices joined at the stem and ending in a globular non-helical head.

The difficult task of identifying receptor proteins with complementary binding sites for C1q is compounded by the highly charged nature of the different domains of C1q. Although newer candidate receptors such as C1qR(p) and CR1 have emerged, full understanding of C1q-C1q receptor interactions is evolving. In view of the diverse functions that C1q may perform, it has been suggested that several C1q-binding proteins may act in concert, as a C1q receptor complex, to mediate and/or amplify C1q-related functions. One such C1q function is to enhance or initiate the phagocytosis of suboptimally opsonized targets. This process may be a critical mechanism in host defense, particularly at early stages of infection/disease when little or no adaptive response is yet present.

C1q binds immune complexes or microbial surfaces to initiate complement activation and generate membrane lytic complexes, opsonins, and anaphylatoxins. In addition to its role in the classical pathway of complement activation, C1q serves other critical immune functions, including facilitating clearance of apoptotic cells and modulating cellular functions within the adaptive immune response (Teh, 2011). The importance of C1q in immune regulation is reflected in individuals who have complete congenital deficiency of C1q who often develop early-onset photosensitive systemic lupus erythematosus (SLE). In such cases, C1q likely aids immunosuppressive mechanisms via its role in enhancing phagocytosis and clearance of apoptotic cellular debris, as the presence of excessive apoptotic cells is a recognized risk factor for autoimmunity. C1q can directly or indirectly opsonize apoptotic cells in an antibody-independent manner to enhance subsequent clearance of these cells (Paidassi, 2008). This biological process is also an important feature of normal tissue development and homeostasis (Perry, 2008), as in the nervous system, where synapses and distal axons are selectively eliminated during both development and pathologic responses through mechanisms sharing features with apoptotic cell removal (Stevens, 2007). Several C1q functions may be mediated by C1q receptors present on the effector cell surface. Identified C1q receptors include C1q receptor for phagocytosis enhancement (C1qRp/CD93), complement receptor 1 (CR1/CD35), calreticulin (CRT/cC1qR), receptor for the globular head of C1q (gC1qR/gC1qbp/p33), CD91/LRP1/A2MR/APOER, and alpha2beta1 integrin (Duus, 20010; Zutter, 2007; Ghiran, 2002). The specific roles of known C1q receptors are not entirely characterized and are sometimes controversial. For example, some previously described C1q receptors, such as gC1qR and cC1qR, are not plasma membrane proteins, and some studies of C1qRp have failed to confirm C1q binding activity (McGreal, 2002). In view of the diverse functions of C1q, distinct C1q receptors may act in different cells or under specific conditions to mediate and/or amplify C1q-related functions.

Phagocytosis is normally accompanied by the generation of a respiratory burst. C1q, like opsonin, can also trigger leukocyte superoxide production by a unique CD18-dependent mechanism. (Goodman, 1995; Tenner, 1982) The involvement of β2 integrins has been demonstrated by the ability of anti-CD18 monoclonal antibodies (mAb) to block the response and the inability of CD18-deficient polymorphonuclear neutrophils (PMN) to respond to C1q. But β2 integrins have not been successfully demonstrated to serve as C1q receptors; instead, they appear provide an essential second signal for leukocyte response. The C1q receptor CR1, although expressed on leukocytes, is not involved in C1q-triggered superoxide production in neutrophils. (Tyagi, 2000) Thus, the C1q receptor(s) responsible for C1q-triggered superoxide production in leukocytes remains unidentified.

The importance of C1q in vivo is demonstrated in C1q deficient individuals. While one anticipates C1q deficiency to render hosts more susceptible to microbial infections, the consistent association of C1q deficiency, in humans and mice, with excessive inflammation and systemic lupus erythematosus (SLE)-like autoimmunity is not directly explained by its classical role in complement activation. (Botto, 1998; Kolble, 1993) In this respect, the ability of the complement system to enhance apoptotic cell phagocytosis or clearance is relevant. (Mevorach, 1998) In C1q knockout mice, marked accumulation of apoptotic bodies occurs. This observation correlates well with the notion that apoptotic cells, when not cleared efficiently, may represent a source of auto-antigens which drive the autoimmune response. The role of C1q in the clearance of apoptotic cells has been highlighted by the finding that C1q binds directly and specifically to surface blebs on apoptotic cells. Further evidence that C1q has important immunomodulatory functions is seen in numerous studies demonstrating that C1q can bind to a variety of pathologically relevant targets in an antibody-independent manner. (Johnson, 2002) As one example, C1q binds directly to synthetic β-amyloid peptides, which may be relevant in the context of Alzheimer's disease.

Although the X-ray structure of RAGE is not known, the primary sequence indicates that the overall structure of RAGE should resemble immunoglobulin structures. The RAGE gene lies within the major histocompatibility complex (MHC) class III region on chromosome 6, where genes play important roles in the immune system and autoimmunity, including the complement system of which several members are located in this region, e.g. C2, C4a, C4b, factor B. These known observations led to the study of RAGE's ability to activate the complement system. The inventors subsequently performed binding studies of RAGE-C1q interaction summarized in Table 2.

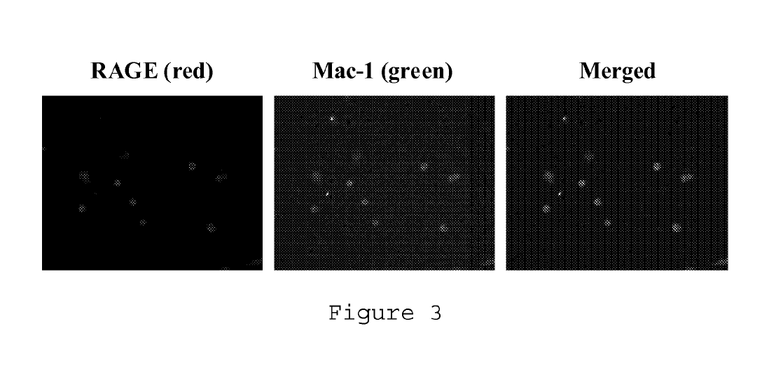

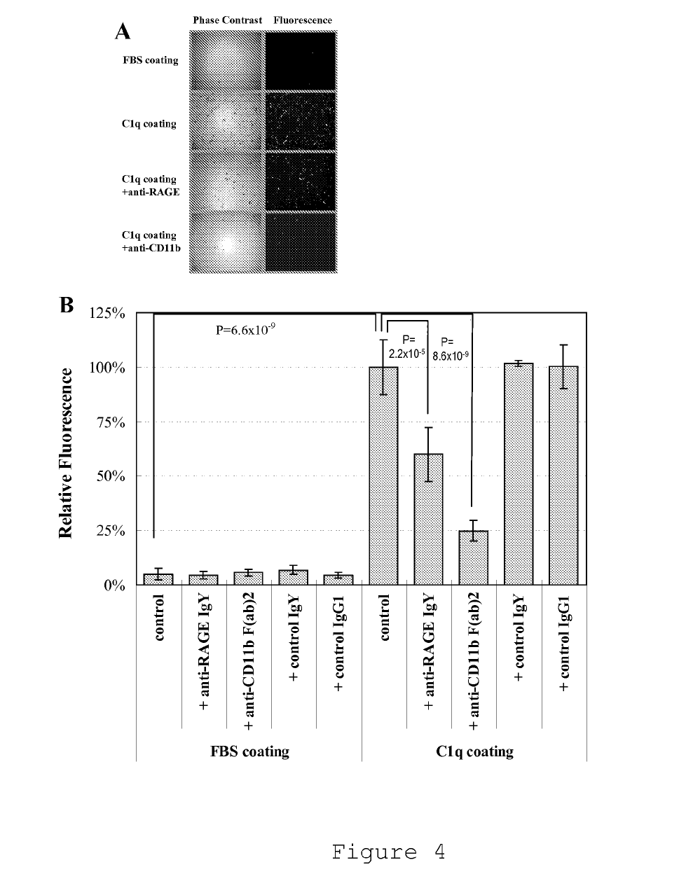

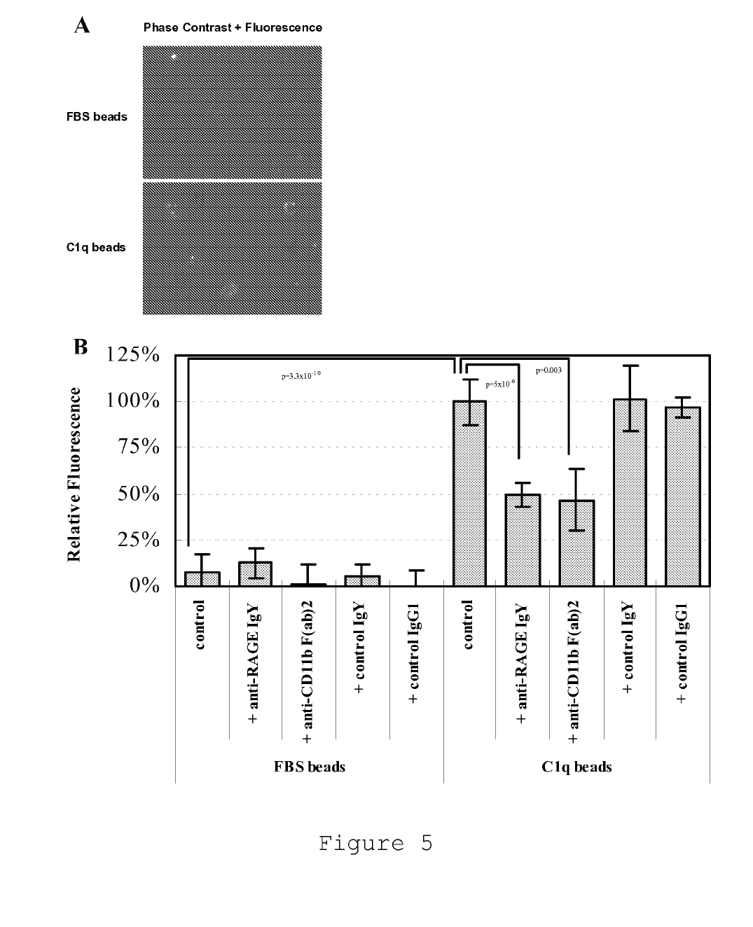

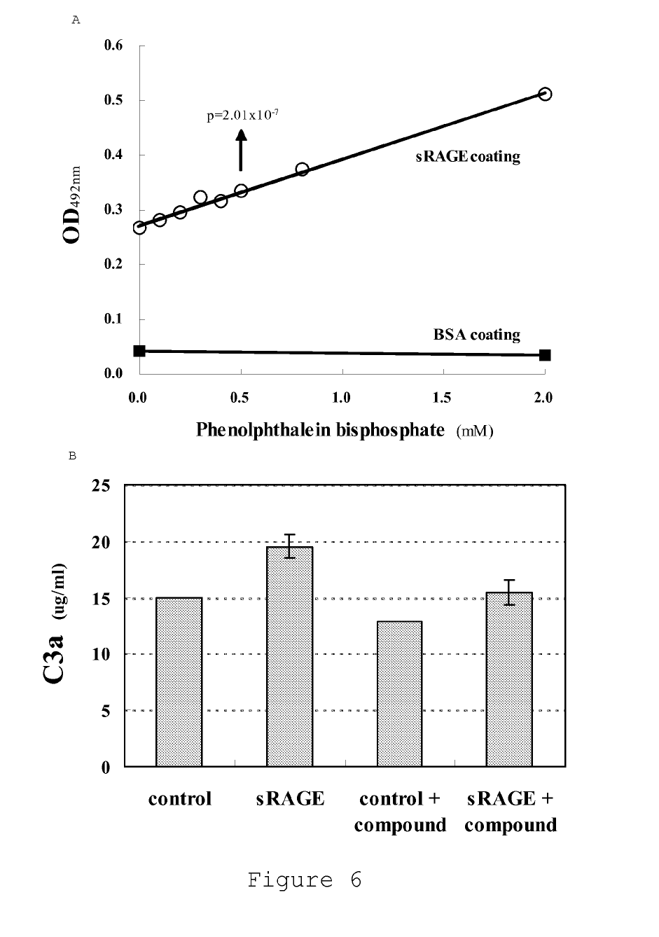

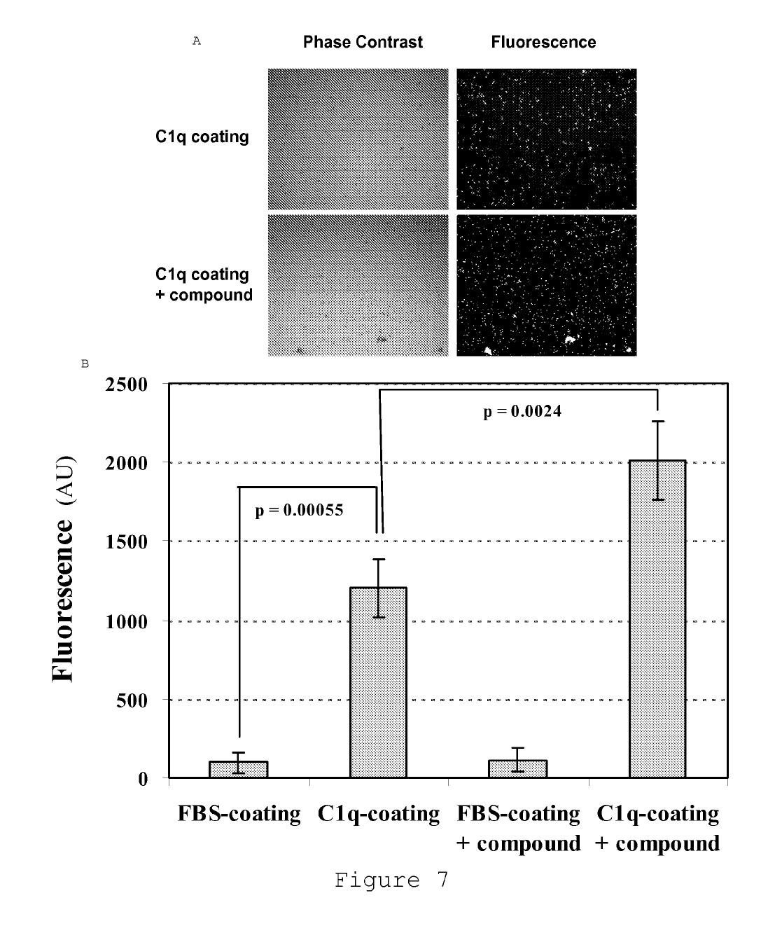

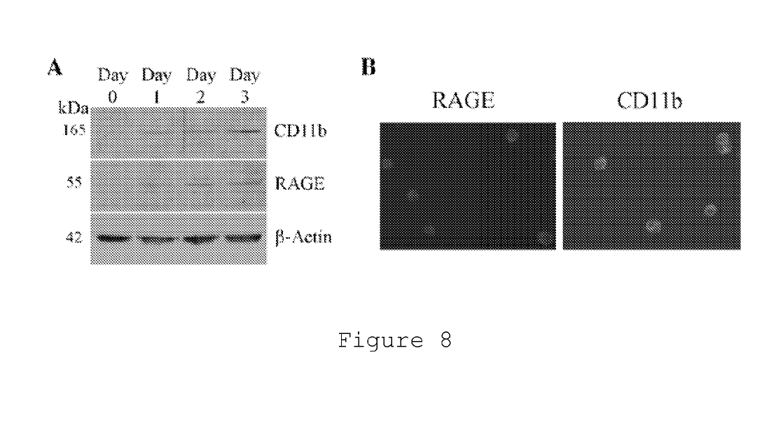

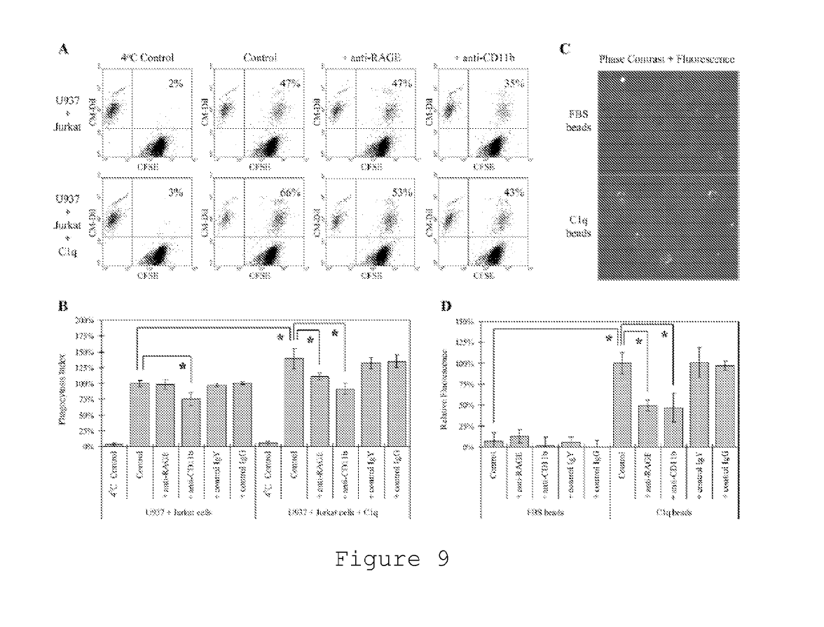

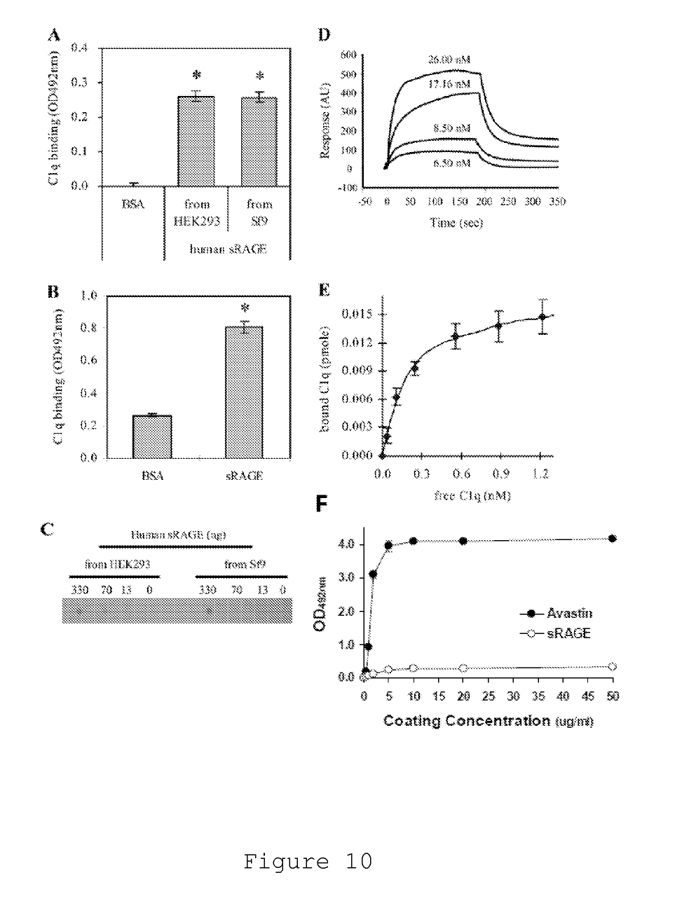

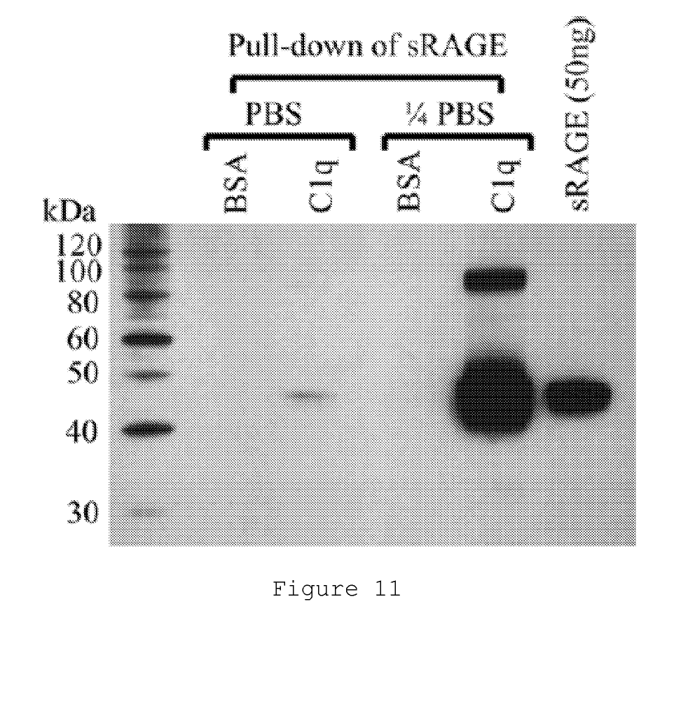

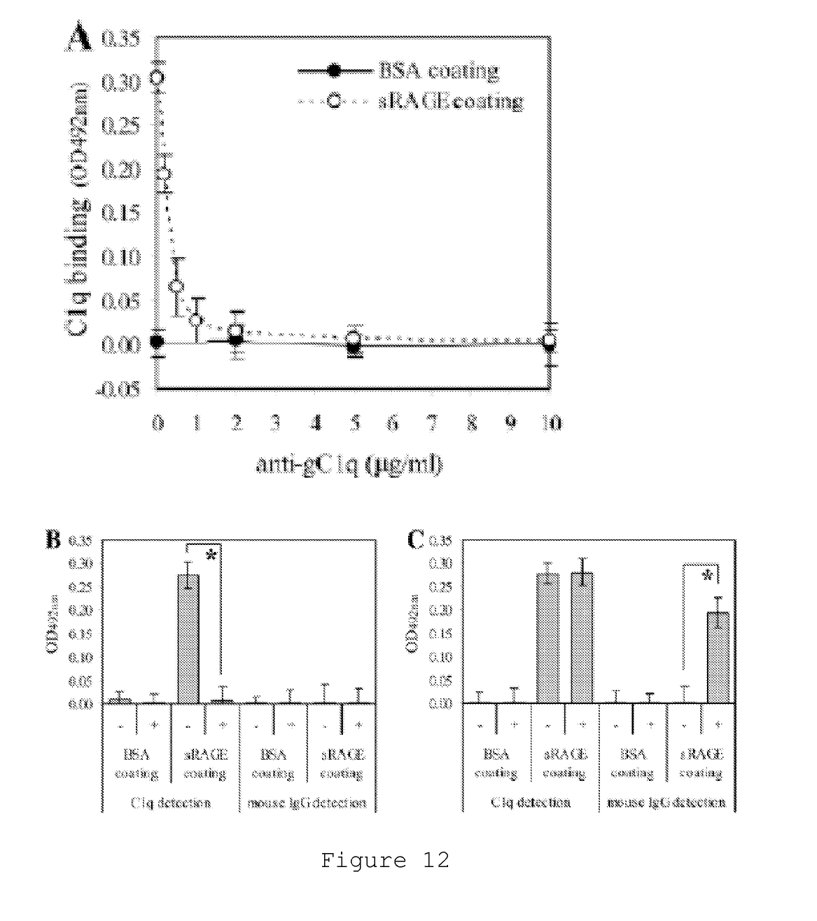

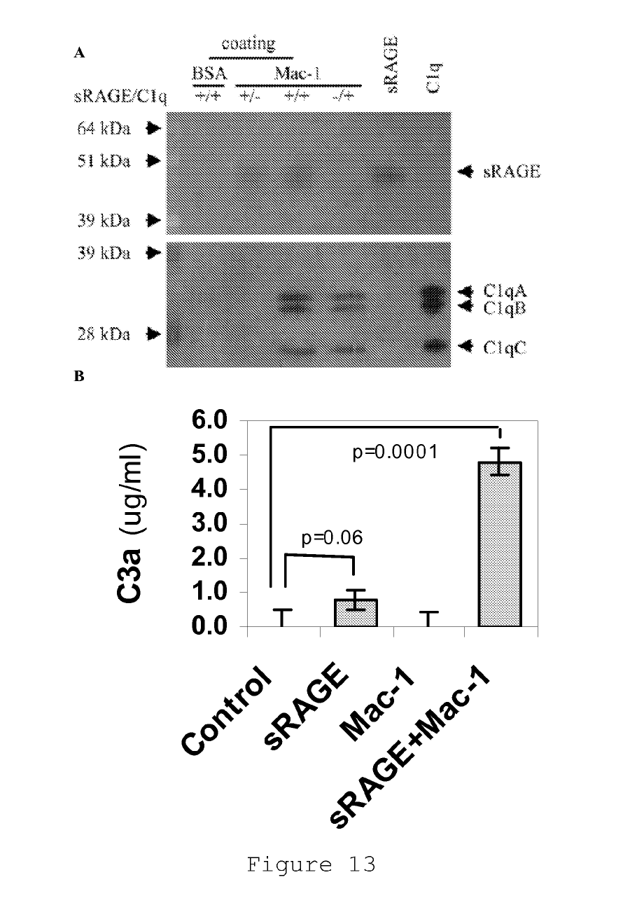

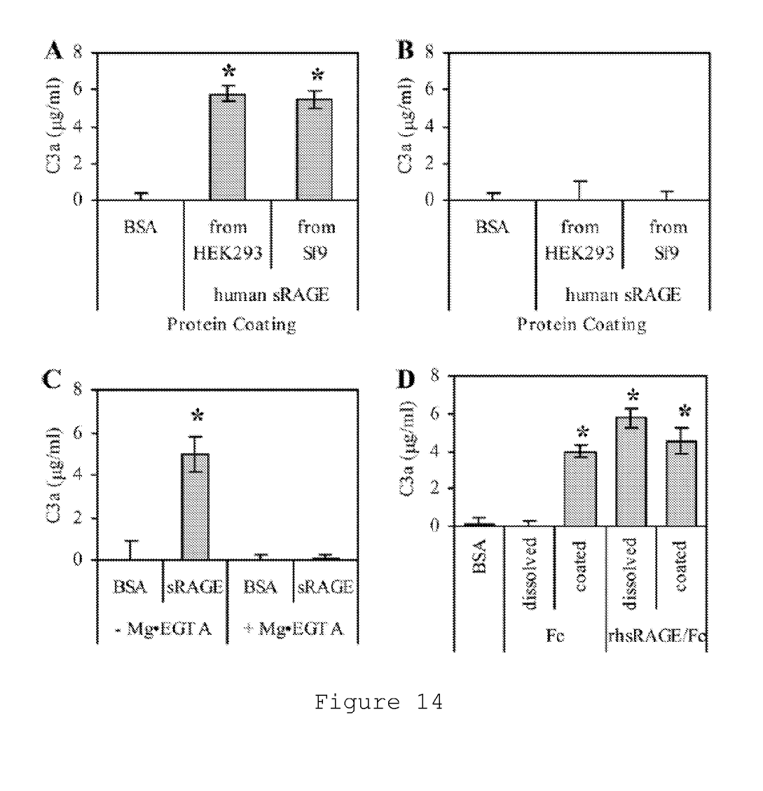

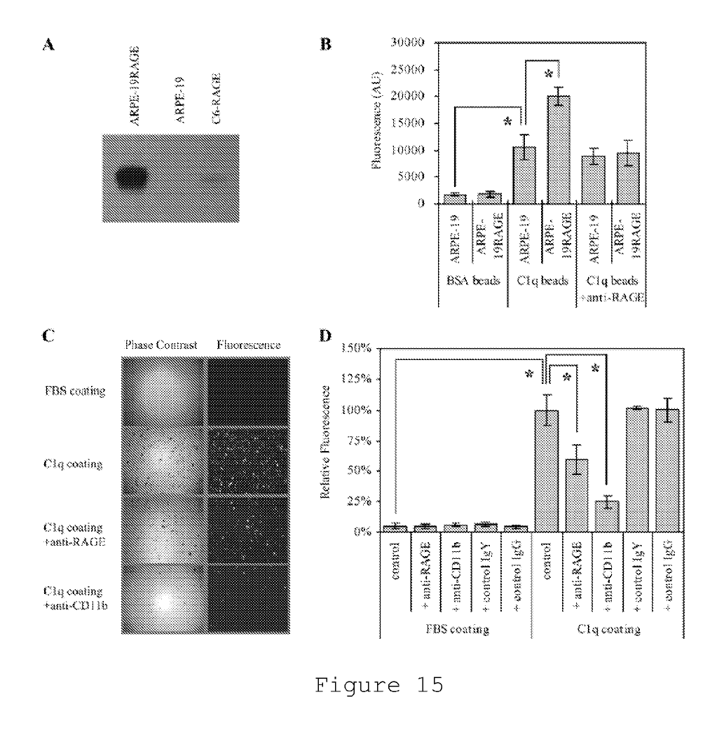

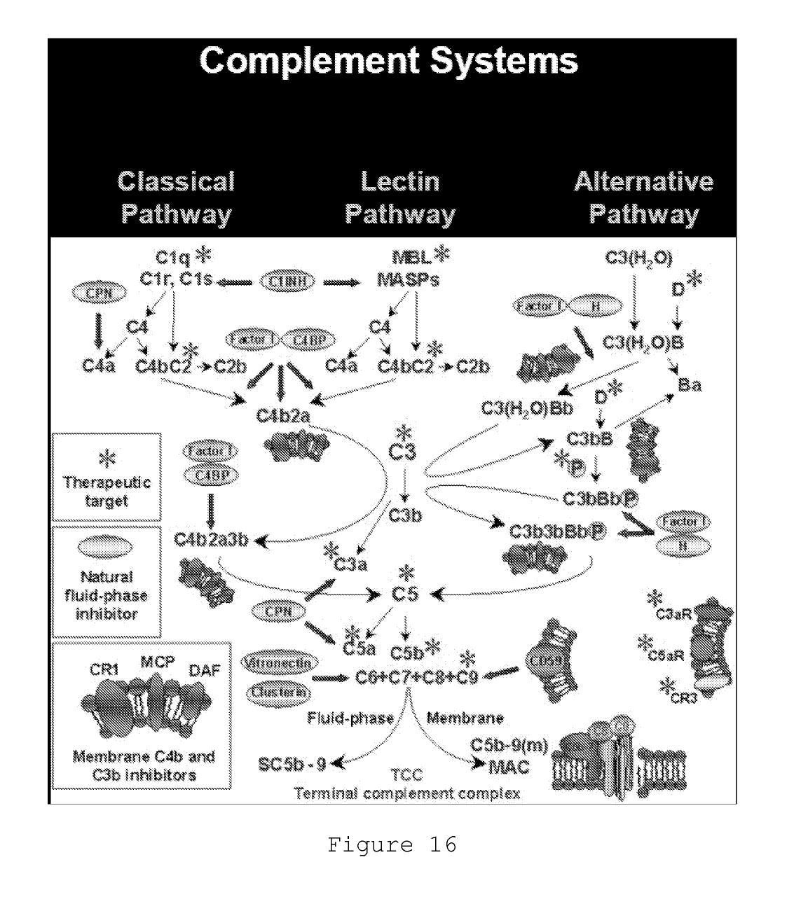

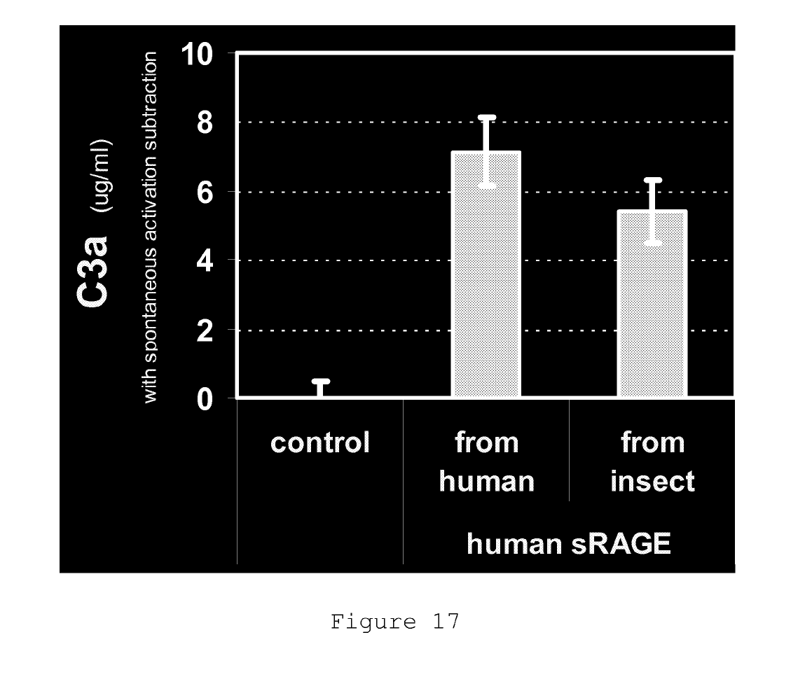

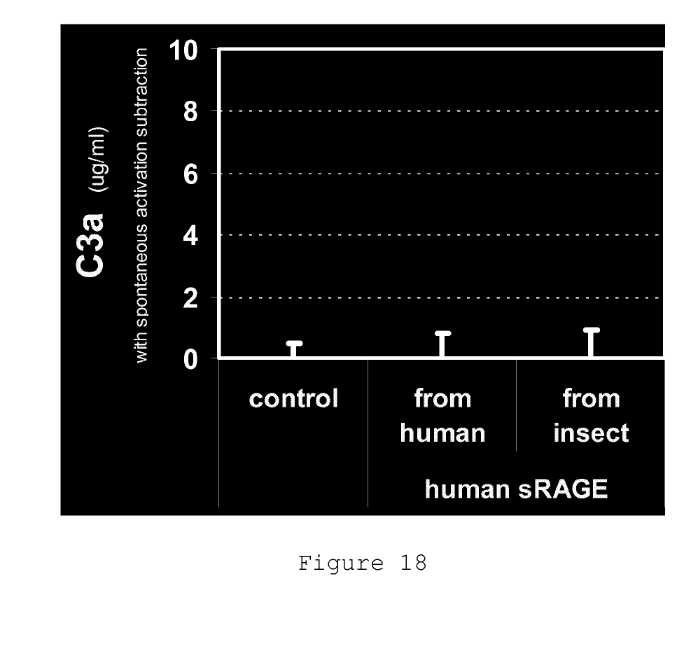

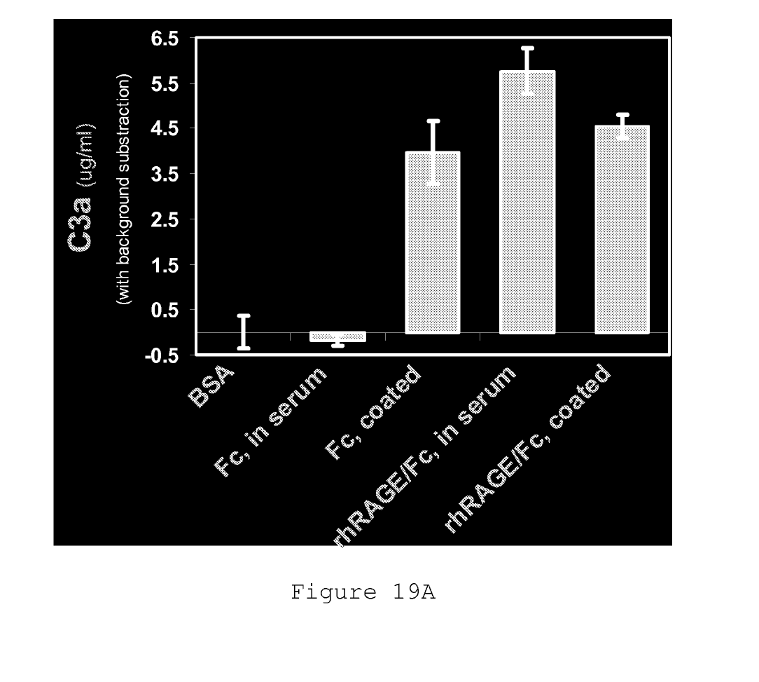

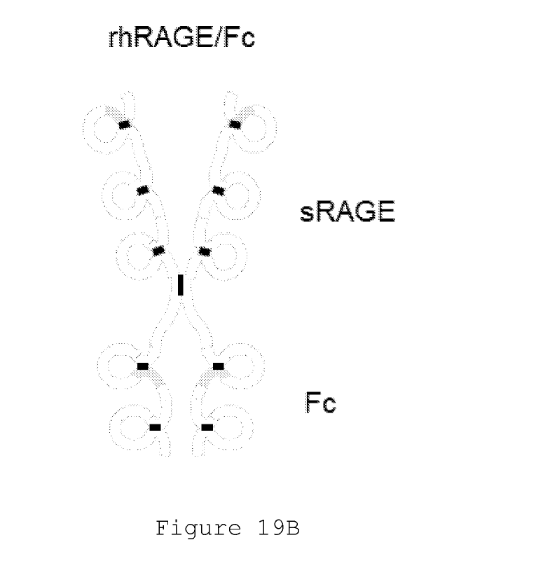

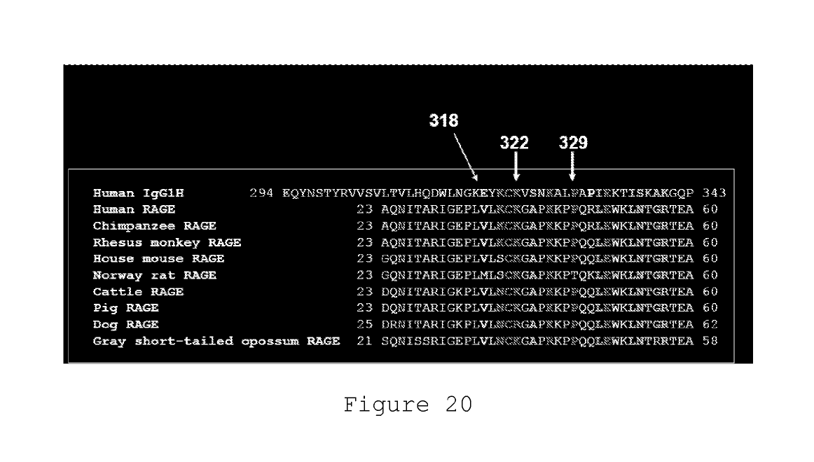

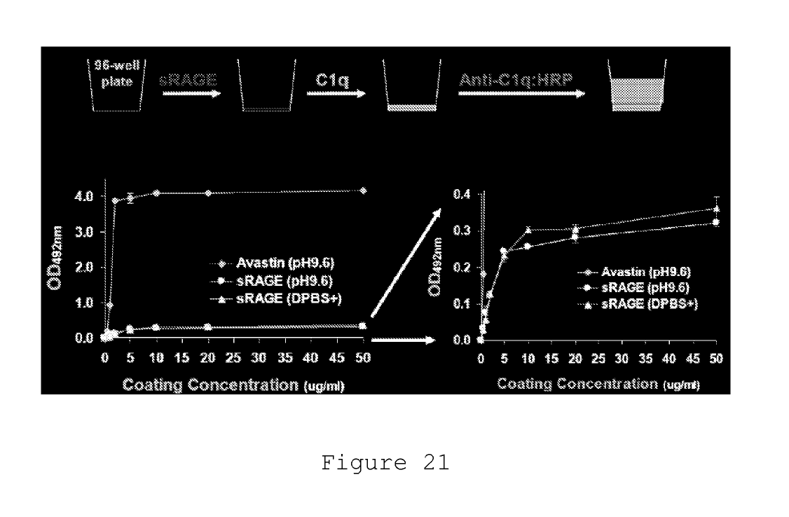

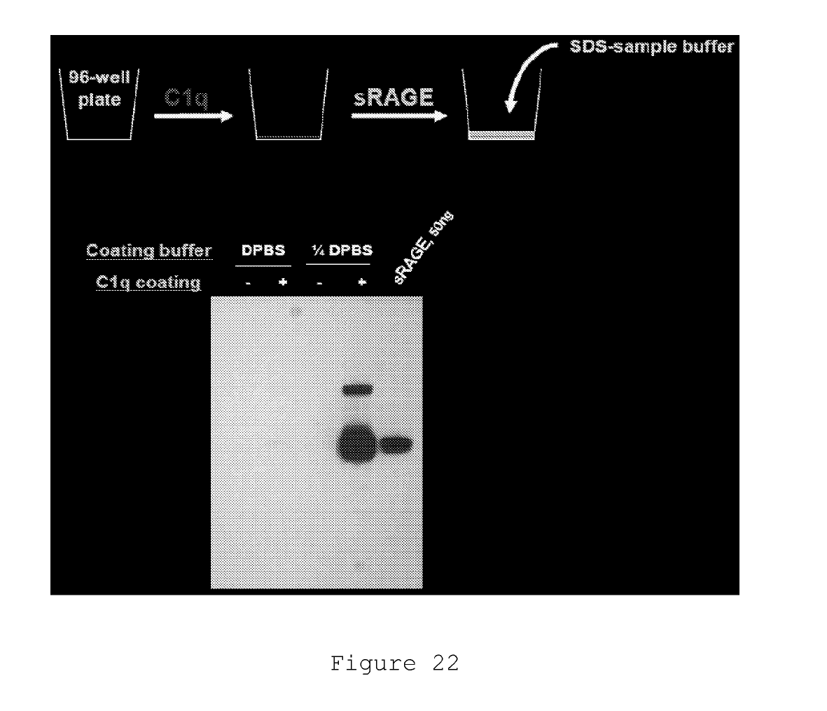

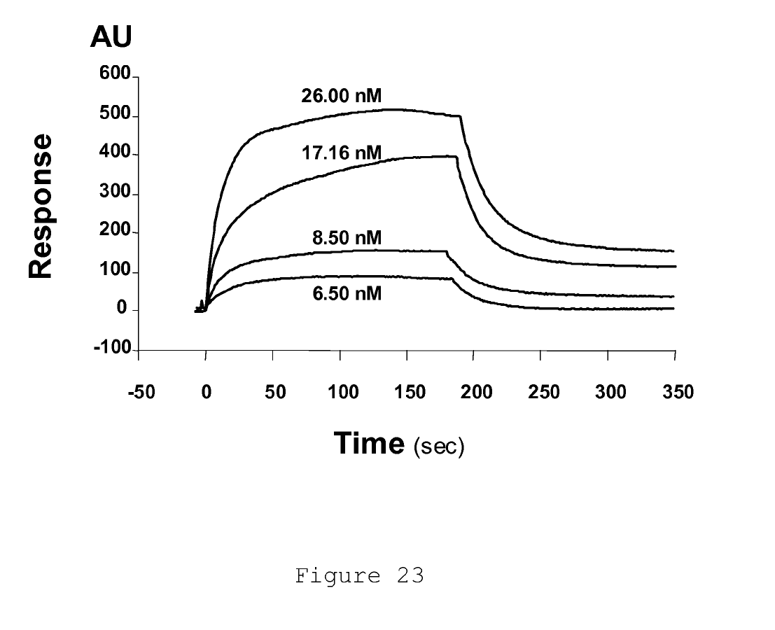

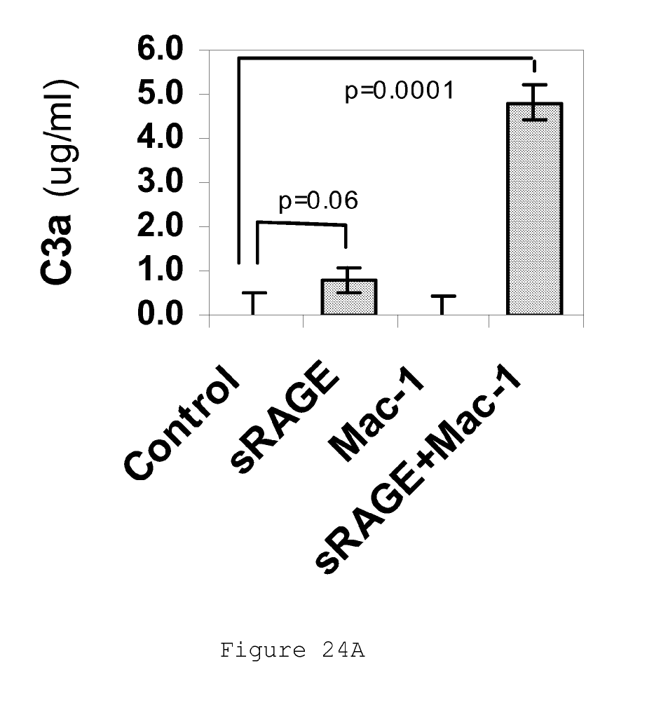

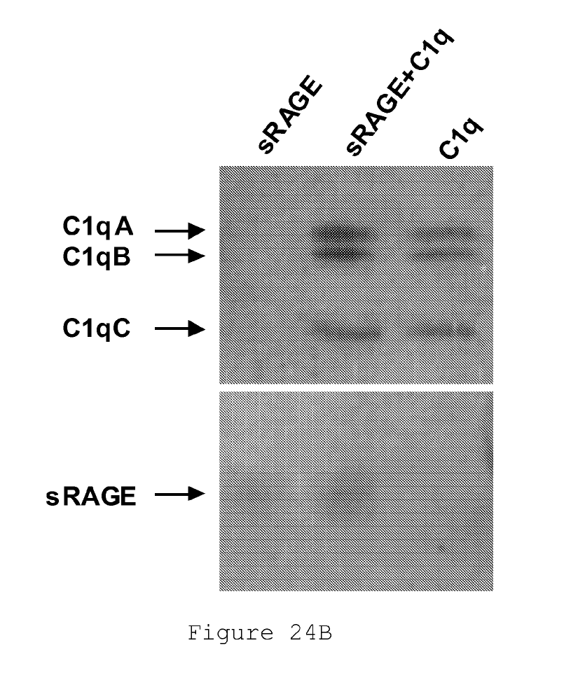

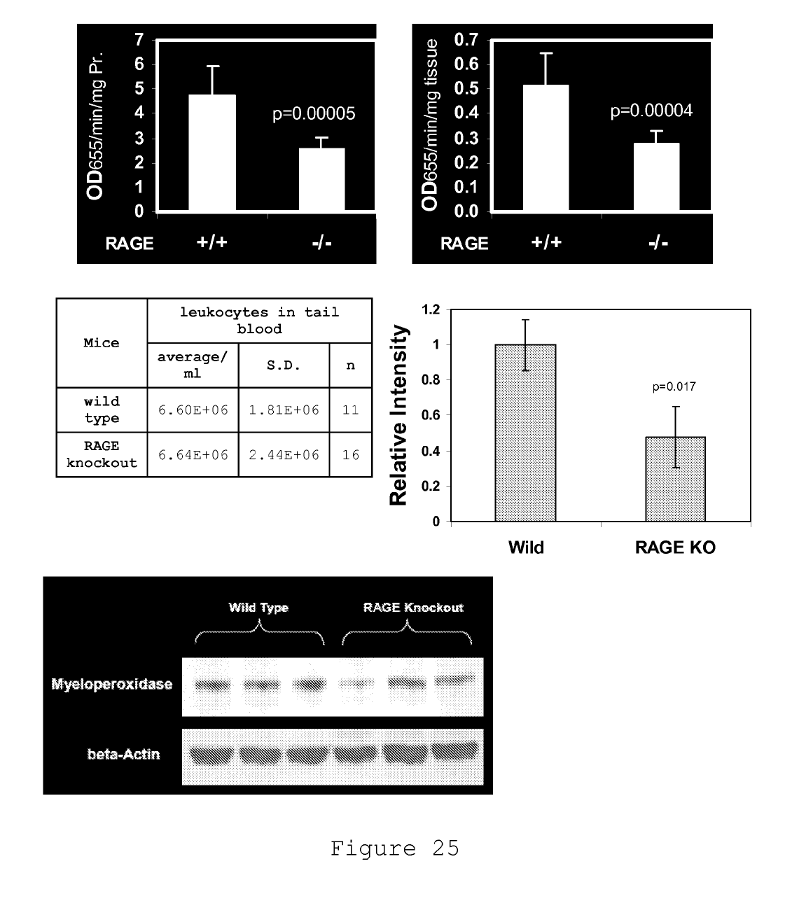

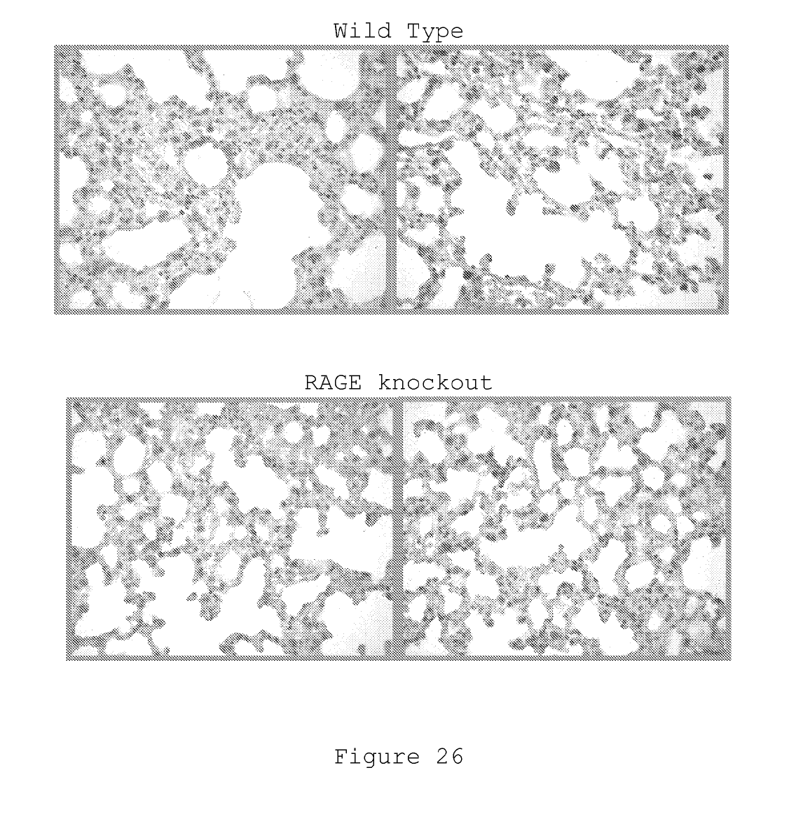

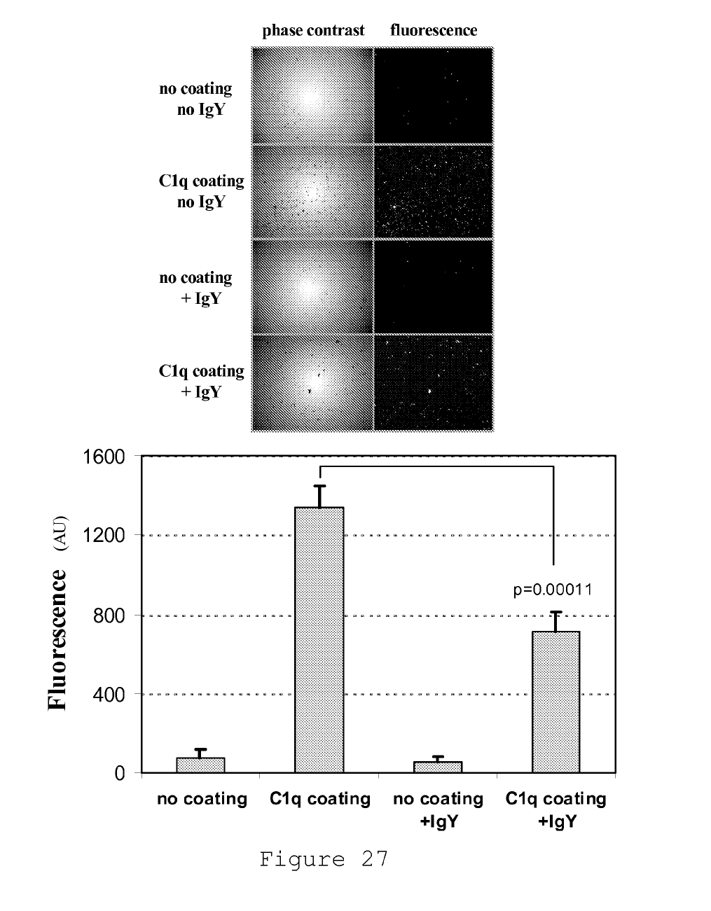

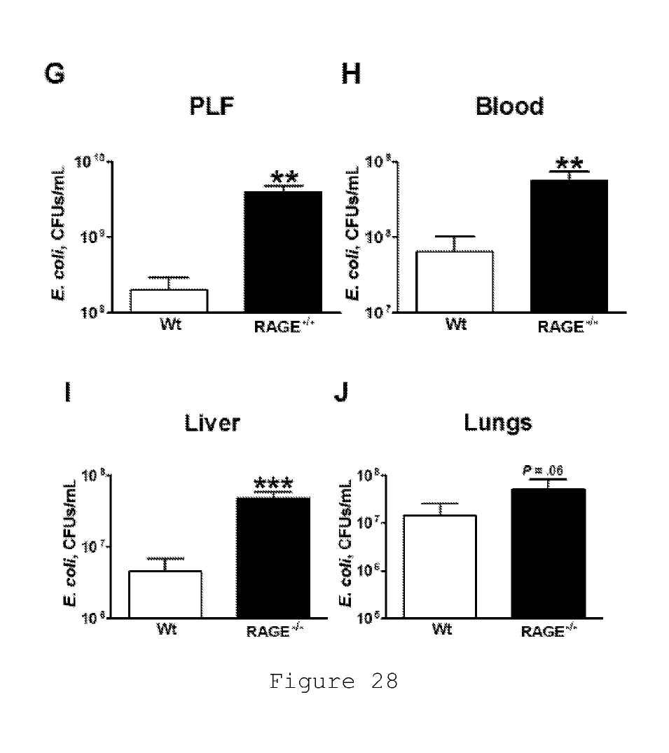

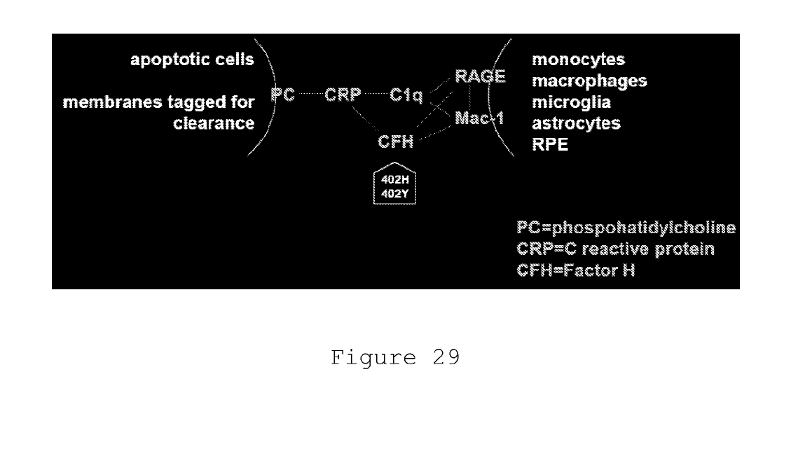

TABLE 2

| Description of Findings | Illustration |
|---|---|
| Immobilized RAGE activates classical complement | FIG. 14 |
| pathway | (A-B) |
| RAGE has ⅛ complement activation activity of | FIG. 1 |
| IgG1 | |
| Multiple binding is necessary for RAGE to | FIG. 14 |
| activate C1q | (D) |
| A possible core C1q-binding site locates at N- | Table 1 |
| terminal of RAGE | |
| Direct RAGE-C1q binding confirmed by pull-down | FIG. 10 |
| experiment and characterized by kinetic study | |
| RAGE-C1q binding is ionic strength-dependent, as | FIG. 11 |
| with IgG and C1q interaction | |
| RAGE-Mac-1 complex has stronger binding for C1q | FIG. 13 |
| and can activate complement system in liquid | |
| phase | |
| Both RAGE and Mac-1 co-localize on monocytes | FIG. 3 |
| plasma membrane | |
| RAGE and Mac-1 dependent monocytes adhesion | FIG. 4 |
| RAGE and Mac-1 dependent monocytes phagocytosis | FIG. 5 |
| Phenolphthalein bisphosphate enhanced RAGE-C1q | FIG. 6 |
| interaction | |
| Phenolphthalein bisphosphate increased monocytes | FIG. 12 |
| adhesion on C1q-coated surface | |

Our first experiment was conducted with assumption that RAGE is an IgG-like molecule. We coated purified RAGE on a cluster plate to mimic the immune complex structure, and we then tested its activity with fresh human serum containing the complete complement system (Quidel, San Diego, Calif., catalog number: A113). To avoid complement activation that would occur due to incompatibility between human serum and RAGE protein, we employed two forms of soluble RAGE (sRAGE, which lacks transmembrane domain and cytosolic tail of full length RAGE) that were both produced by the human RAGE gene, one expressed in insect cells (sf9) (obtained from Ann Marie Schmidt's lab, New York, N.Y.) and the other expressed in human kidney cells (HEK 293) (purchased from BioVendor, Minneapolis, Minn., Catalog number: RD172116100-HEK). We determined that either form of sRAGE was capable of activating complement systems with comparable activity (FIG. 14A). For controls, we tested coatings of BSA (bovine serum albumin), HSA (human serum albumin) and heat-treated human serum, all of which resulted in the same basal complement activation that was subtracted from experimental data (data not shown). To characterize the complement pathway that was activated by sRAGE, we repeated the experiment with C1q-depleted human serum (Quidel, San Diego, Calif., catalog number: A509). As shown in FIG. 14B, both forms of sRAGE lost the ability to activate the complement system, indicating that sRAGE interacts with C1 and activates the classical pathway of complement system under these conditions.

We next tested the ability of Avastin (Genetech, San Francisco, Calif.), a humanized IgG1 antibody for VEGF that is clinically employed in oncology and, off-label, in neovascular ocular disease, to form C3a under these conditions, comparing it with sRAGE. Avastin was about 8-times more active than sRAGE at every concentration used for coating studies that approached saturation between 10 to 20 μg/ml for both proteins (see FIG. 1).

Another available RAGE molecule is a fusion protein that combines the extracellular domain of human RAGE and the Fc region of human IgG1 via a peptide linker. This chimeric protein is expressed in a mouse myeloma cell line, NSO. The recombinant mature human RAGE/Fc chimera (rhsRAGE/Fc, R&D Systems, Minneapolis, Minn., catalog number: 1145-RG) is a disulfide-linked homodimeric protein that has at least three potential C1q-binding sites, two on RAGE extracellular domains and one on the Fc fragment of IgG1. When compared with the homodimeric recombinant mature human IgG1 Fc that also has peptide linker attached, this fusion protein displayed distinct activity upon complement activation (see FIG. 14D). Immobilization of both proteins activate the classical complement pathway, as expected, while, in contrast to the Fc dimer, rhsRAGE/Fc dimer in solution also activated complement. This data suggests that multivalent binding of RAGE molecules with C1q is necessary for complement activation.

It has been demonstrated that the core C1q binding site on IgG molecules is ExKxK, with replacement of amino acid E with V reducing its binding affinity to one tenth. (Duncan, 1988) The RAGE sequence analysis suggests that there is a core C1q-binding site on its N-terminal residues 35-39, VLKCK (please note that residues 1-22 are the signal peptide for RAGE). Table 1 shows the highly matched region surrounding this core C1q binding site between human IgG1H and RAGE from different species. It is important to note that, in addition to the matched core C1q binding site, other residues that have been reported to be important for C1q binding are also matched, such as 321C, 329P in IgG1H and 38C, 46P in RAGE. (Howes, 2002; Kaul, 1997). It is also important to note that both sequences in this region have crowded, charged residues and most of them match perfectly, indicating that electrostatic attraction likely plays an important role in C1q-sRAGE interaction as it does in C1q-IgG interaction. In addition, both IgG1H and RAGE have N-linked oligosaccharides located near their core C1q binding site, e.g. 297 N for IgG1H and 25 N for RAGE (Duncan, 1988; Gouras 2008; Kaul, 1997; Neeper, 1992.) that probably contribute to the recognition and binding of C1q.

As with antibody molecules, sRAGE monomers are not active on complement activation when they are in solution (FIG. 13B). Also consistent with data in FIG. 14D, at least two binding sites are needed in order to alter C1q conformation to lead to the activation of C1r which in turn activates C1s. Mac-1 is a β2 integrin, and several studies indicated that it may have binding affinity for the collagen domain of C1q (Goodman, 1995; Lauvrak, 1997). With consideration of RAGE's global domain binding ability on C1q, one might consider that RAGE and its ligand Mac-1 may generate a complex structure that can activate C1 if they do not block their respective C1q binding sites in the complex structure. To test this hypothesis, we added preincubated sRAGE and Mac-1 (purchased from R&D Systems, Minneapolis, Minn., catalog number: 4047-AM) solution into fresh human serum and assayed complement activation. As expected, sRAGE or Mac-1 alone, in liquid phase, could not activate the complement system, but their mixture did, as shown in FIG. 13B. To further confirm the interactions between sRAGE, Mac-1 and C1q, we performed a pull-down experiment with immobilized Mac-1 as bait. As shown in FIG. 13A, Mac-1 pulled down sRAGE and C1q when they were incubated separately with Mac-1, and the pull-down amount of both proteins increased when they were incubated together

As shown in FIG. 13, RAGE and Mac-1 can form a complex structure with enhanced binding affinity for C1q compared to RAGE or Mac-1 alone. Monocytes and macrophages express both RAGE and Mac-1 proteins on their plasma membrane (see FIG. 3); therefore, these cells are attractive target cells to initially study the function of RAGE and Mac-1 complex upon monocyte and macrophage physiological functions that involve the C1q molecule. The inventors' first effort to study C1q-induced monocyte adhesion and phagocytosis did not definitely demonstrate the involvement of RAGE and Mac-1 complex. In those experiments, phorbol-12 myristate 13-acetate (PMA) was employed to activate/differentiate the human monocyte cell line U-937. Subsequent experiments with Vitamin D₃ and Transforming Growth Factor-β (TGF-β) as activators of U-937 cells dramatically demonstrate that RAGE antibody displays a strong inhibitory effect on C1q-induced U-937 cell adhesion and phagocytosis, as shown in FIGS. 4 and 5. To minimize binding of antibody to Fc receptors and complement component proteins, chicken IgY antibody against human RAGE (obtained from Ann Marie Schmidt's lab, New York, N.Y.) and F(ab′)₂ of anti-human CD11b antibody (purchased from Sigma, St. Louis, Mo., catalog number: C0551) were used in these experiments. This data links the molecular interaction of C1q and RAGE to native cellular function, specifically the recruitment of activated monocytes and phagocytosis of C1q-coated beads, and is further proof of concept of hypotheses outlined in the proposed application.

### Phenolphthalein Biphosphate Enhanced RAGE-C1q Interaction and Inceased Monocyte Adhesion on C1q-Coated Surface

Chemicals and biomolecules that can enhance or weaken the interaction of RAGE and C1q have potential applications for therapeutic purpose. One such chemical is Phenolphthalein Biphosphate or Phenolphthalein bisphosphate tetrasodium salt (purchased from Sigma, St. Louis, Mo., catalog number: P9875). It can enhance RAGE-C1q interaction with dose-dependent manor (See FIG. 6A), but without enhancement on complement activation (see FIG. 6B), which was expected according to Svetlana Bureeva's studies (Bureeva, 2005), the contents of which are hereby incorporated by reference.

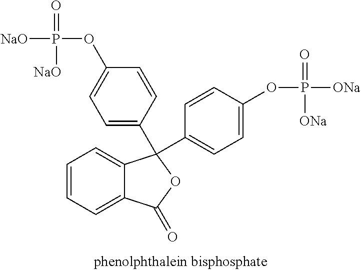

Phenolphthalein bisphosphate has been used in bacteria culture media as phosphatase substrate. It was tested with human monocyte cell line U-937 cells, but no cytotoxicity at 2 mM was found, and it couldn't activate or differentiate U-937 cells either. Because it could enhance RAGE and C1q interaction, we tested its effect on monocytes adhesion to immobilized C1q. As shown in FIG. 7, 2 mM of phenolphthalein bisphosphate in medium could significantly increase the adherent U-937 cells. Phagocytes are major protective cells in the body by ingesting harmful foreign particles, bacteria, and dead or dying cells. In an in vivo abdominal sepsis model caused by intraperitoneally injection with Escherichia coli, RAGE was involved in defense against the growth and/or dissemination of E. coli (The Journal of Infectious Diseases 2009, 200:765-773). In the presence of RAGE, E. coli numbers were much lower in the injection site as well as in major organs of the mouse. With the help of phenolphthalein bisphosphate, we expect that RAGE's function in this sepsis model could be enhanced further.

### Example 3

### Early AMD RPE Cells have Heterogeneous Expression of S100b with Plasma Membrane and Nuclear Distributions

### Materials and Methods for Example 3.

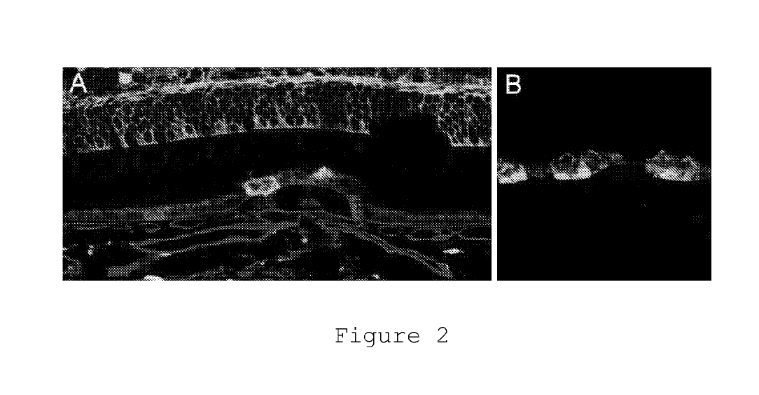

A retina section was prepared from 23-24 years old femal rhesus monkey with moderately severe drusenoid maculopathy. (See FIG. 2A). A retina section was prepared from a 53 year old man with type II diabetes. (See FIG. 2B). All sections were immunostained with s100 antibody and Alexa Fluor 488-labeled secondary antibody. Green signal is S100 protein and the red signal in RPE cells is autofluroescense.

### Results and Discussion for Example 3

Several RAGE ligands are identified in Bruch's membrane deposits and drusen, including AGEs, amyloid and s100 proteins. RAGE itself is also upregulated in RPE cells adjacent to these deposits. Preliminary immuofluoresnce studies were performed on human and monkey specimens. As shown in FIG. 2, Muller cells that natively express RAGE also expressed high level of S100 protein. Some individual RPE cells also expressed S100, especially those RPE cells abutting upon drusen deposits. A special feature of S100 expression in RPE cells was its presence in plasma membrane and nuclear translocation.

### Example 4

### Rage-Mediated Immune Mechanisms in Retinal Disease

Advanced age-related macular degeneration (AMD) remains the leading cause of vision loss in the United States. Genetic studies of AMD suggest strong roles for perturbations in the immune system, particularly complement factors, in the pathogenesis of this disorder.

This study focuses upon immune mechanisms mediated by RAGE (Receptor for AGEs [advanced glycation endproducts]) in native homeostasis and in outer retinal disease. Protein ligands that bind RAGE and increased expression of this cellular receptor are present in early and late forms of AMD. Using several experiments, the inventors have determined that RAGE is a complement (C1q global domain) receptor and, under certain conditions, can bind C1q and activate C1 complex. An improved understanding of the biology of RAGE/C1q interaction, related immune mechanisms, and aberrant regulation of these processes can provide insights into their contribution(s) to inflammatory dysfunction in conditions such as AMD.

To characterize such RAGE-mediated immune perturbations, the inventors performs biochemical studies of RAGE/C1q interaction and utilizes RAGE-gene targeting techniques to address 3 important topics: (1) the ability of RAGE to function as C1q-binding protein in complex structure and the role of complement inhibitors in RAGE/C1q interaction (2) the conditions that RAGE-bearing cells can activate the complement system (3) the native biologic roles of RAGE/C1q interaction on complement-enhanced and complement-dependent functions. The inventors also perform immunochemical imaging and EM studies in aged monkey retinal specimens with drusen to assess the relevance of the properties of RAGE and its ligands in the aging macula at risk for advanced AMD. This study provides insights into the native role of RAGE/C1q interaction in complement-mediated immune mechanisms, and, in the case of AMD, better defines the contribution of RAGE in the development of outer retinal diseases that result from deficient immune regulation.

Abnormalities in regulation of the complement cascade and associated inflammatory triggers that accompany the development of age-related macular degeneration (AMD) suggest that local inflammatory events critically affect the response of the outer retina to superimposed biochemical and metabolic stresses in the aging macula. Proteomic studies in the aging macula have identified several ligands for RAGE (Receptor for AGES [advanced glycation endproducts]) in drusen deposits, among them AGEs, amyloid-β peptide, 5100 protein, and CD11b. Previously it was demonstrated that specific activation of RAGE on RPE cells induces cell proliferation and upregulated the expression and secretion of VEGF, the major growth factor responsible for choroidal neovascularization (CNV). RAGE is a multiligand signal transduction receptor of the immunoglobulin superfamily of cell surface molecules that mediates a spectrum of distinct cell-specific effects in the setting of its ligands. RAGE was first identified as a receptor for AGEs, heterogeneous groups of products of nonenzymatic glycation and oxidation of amino groups of proteins and lipids that develop during normal metabolic aging and that accumulate more rapidly in the setting of hyperglycemia, oxidant stress, and inflammation. RAGE also binds non-AGE ligands implicated in other disorders, including neurodegenerative diseases (amyloid-β peptide) and acute and chronic inflammatory conditions (S100/calgranulins, a family of proinflammatory cytokines; high mobility group box 1 [HMGB1] or amphoterin; and the leukocyte integrin Mac-1). As research increasingly links visually threatening retinal disorders to inflammatory mechanisms, it is worthwhile to investigate the specific biologic roles of RAGE and its ligands in the initiation and propagation of immune responses including binding and activation of complement and leukocyte recruitment and function. In recent efforts to characterize RAGE's role in inflammatory mechanisms, it was determined that RAGE is a native C1q global domain receptor that, under certain conditions, can activate the classical pathway of complement system via binding with C1q and activating C1 complex. This example focuses upon the newly identified C1q-binding activity of RAGE to study relevant functions and mechanisms associated with such interaction in homeostasis and in the setting of other RAGE ligands that accumulate in the aging macula. We hypothesize that the native biological roles of RAGE and C1q interaction include leukocyte recruitment and function such as C1q enhanced phagocytosis and macrophage-mediated apoptotic cell clearance. In the setting of inefficient complement inhibition such as CFH (complement factor H) mutations, we hypothesize that excessive RAGE-mediated complement activation, at least in part via ligand-triggered formation of complex structures, initiates and/or amplifies cellular dysfunction through enhanced complement-dependent cytotoxicity of RAGE-bearing cells such as macrophages and RPE cells in AMD. This invention involves the indentification of undiscovered RAGE-mediated biological processes in normal physiology and their contributions to inflammatory disease in the aging macula.

Gene polymorphism studies indicate that complement activation contributes to age-related macular degeneration (AMD): Complement Factor H (CFH); Complement Factor B; C2; C3; etc.

Many studies also indicate that RAGE was involved in AMD. Many RAGE-binding proteins were found in drusen and Bruch's Membrane in AMD patients. RAGE itself was also present at AMD lesion site. Transgenic mice over-expressing RAGE ligand S100B develop AMD-like pathology.

Examples of RAGE-binding Proteins are as follows: Advanced Glycation Endproducts (AGEs): 5100 Proteins; Amyloid; HMGB1; Mac-1; and C1q.

Mac-1 mainly expresses in leukocytes. RAGE highly expresses in endothelial cells of lung. Lung has about 100 times more leukocytes density in its blood than other peripheral blood.

RAGE and its Ligands in the Retina.

In the retina, RAGE has been identified within several cells of the neural retina but particularly Müller cells, vascular endothelial cells, and RPE cells. (Barile, 2005; Howes, 2004; Kaji, 2003; Soulis, 1997). In studies focusing upon physiologic effects of diabetes, it is evident that AGEs are capable of inducing significant perturbations in several cells of the retina and that retinal RAGE is upregulated in the diabetic retina, priming it for the neurovascular dysfunction that develops in diabetes mellitus. In a murine model of diabetic retinopathy, treatment with soluble RAGE attenuated neuronal dysfunction assessed by electroretinography and suppressed the development of acellular capillaries and pericyte ghosts (Barile, 2005). Studies in RAGE transgenic mice, employing a flk-1 promoter that increases expression of RAGE in the systemic vasculature including the retina, demonstrate enhanced development of vascular events seen in diabetic retinopathy, including increased blood-retinal barrier breakdown and retinal vascular leukostasis, with these disturbances particularly pronounced in diabetic RAGE-transgenic mice. The precise contribution of AGEs, non-AGE ligands, and RAGE, independent of and/or in concert with other hyperglycemia-induced biochemical mechanisms, at the various stages of diabetic retinopathy remains under investigation. Soluble forms of RAGE and the RAGE ligands, AGEs, S100/calgranulins and amphoterin, are also detected in studies of the vitreous fluid of eyes with proliferative retinal disease, while minimal to no evidence of them is identified from control eyes with macular hole. Similarly, in proliferative epiretinal membranes (ERMs) removed at surgery, RAGE is consistently expressed in spindle cells that suggest glial origin, likely representing the presence of activated and differentiated Müller cells. In diabetic ERM specimens, RAGE is also expressed in vascular cells consistent with endothelial expression of RAGE. Müller cell studies in vitro confirm that incubation of these cells with both AGEs and non-AGE ligands result in activation of intracellular signalling pathways typical of RAGE activation (unpublished data), though the cellular effects of this activation have not been characterized.

Outer retinal perturbations including photoreceptor and RPE dysfunction and alterations in Bruch's membrane and the choriocapillaris are present in the atrophic and neovascular forms of AMD. Bruch's membrane, as an extracellular matrix with collagen as a major component, is a prime target for AGE modifications. AGEs may exert local biologic effects by receptor-independent or receptor-dependent pathways. In the former case, AGEs may directly impact on the structural integrity of the basement membrane, disrupting matrix-matrix and matrix-cell interactions. Aged human Bruch's membrane contains increased protein crosslinks and accumulates AGEs, impacting its anatomic integrity and potentially impeding its physiologic function (Handa, 1999). Proteomic analysis of drusen identifies several RAGE ligands among the components of these deposits, including AGEs, amyloid-β peptide, 5100 protein, and CD11b (Anderson, 2004; Dentchev, 2003; Glenn, 2009; Gu, 2008; Handa, 1999; Ishibashi, 1998; Johnson, 2002; Seth, 2008). In proteonomic studies of Bruch's membrane, 5100 was one of the more abundant proteins identified in all stages of AMD (Gu, 2009). In the normal human retina, little to no immunolabeling for RAGE is present in the photoreceptor and RPE cell layers. However, when small drusen are present, RAGE was identified in the RPE or both the RPE and photoreceptors. In early AMD and advanced AMD with geographic atrophy, the RPE and remnant photoreceptor cells showed more intense RAGE immunolabeling (Howes, 2004). Consistent with the known biology of RAGE, gene expression studies demonstrated significant upregulation of RAGE mRNA in microdissected RPE cells overlying basal deposits compared to cells attached to morphologically normal Bruch's membrane (Yamada, 2006). In subfoveal membranes of patients with AMD, CML-like immunoreactivity was found in all specimens adjacent to or colocalized with RAGE, but it was not detected in idiopathic membranes (Hammes, 1999). In S100B transgenic mice, autofluorescent granules accumulated early in RPE cells, with loss of photoreceptors, loss of RPE cells, and disorganization of outer segments seen in 6-month old mice (Howes, 2002). In one mouse model of early AMD, studies in neprilysin gene-disrupted mice revealed an accumulation of Aβ in subepithelial deposits and within RPE cells overlying subepithelial deposits. While neovascular events were not observed, Aβ accumulation upregulated VEGF and downregulated PEDF in the RPE in this model (Yoshida, 2005). Whereas A β aggregates bind the C2-Ig-type RAGE domain, Aβ oligomers (believed to be the more pathogenic species) bind to the V-type domain of RAGE (Sturchler, 2008). These findings suggest the possibility that the more aggregated ligand species are those most apt to bind to RAGE and stimulate signaling, particularly consequent to interaction with the V-type Ig domain. In human RPE cells, we demonstrated that RAGE activation by its ligands increases release of VEGF in a NF-kappaB dependent manner. We also determined that soluble S100B and soluble Aβ were less able to activate NF-kB and increase VEGF expression compared to oligomeric forms of these species. These cumulative studies implicate RAGE and its ability to initiate or amplify the consequences of AGE and non-AGE ligand accumulation in the retina as a pathway capable of mediating several cellular perturbations in the retina.

### Complement Activation in AMD.

Though the precise interplay between genetic predisposition and environmental triggers that contribute to the development of AMD remains unclear, specific mutations in the alternative complement cascade are present in a large proportion of cases of AMD, implicating inflammatory pathways in the initiation and/or progression of the disorder (Hageman, 2001; Johnson, 2002; Mullins, 2000). Histological studies have identified the presence of complement components both within drusen and along the RPE-choroid interface in AMD eyes (van der Schaft, 1993). Abnormal regulation of the complement cascade at a local level within Bruch's membrane, drusen and adjacent retinal pigment epithelial cells results in amplified complement activation and influences RPE cell responses. The high rate of metabolism of the photoreceptor transduction cascade and the strong light exposure contribute to outer retinal oxidative stress, which itself has been shown to be active upon complement systems. Accumulation of A2E and other metabolic products are also capable of triggering complement activation (Zhou, 2009). AGEs and other RAGE ligands accumulate in this environment, can be further enhanced during oxidative stress, and may contribute to complement activation. Double immunolabeling experiments confirm the co-distribution of iC3b and Aβ in Aβ-positive vesicles in drusen, but their localization does not overlap, suggesting bridging molecules may exist to connect these structures (Johnson, 2002). Endogenous activators of the complement system normally interact with complement inhibitors, C4BP and CFH, resulting in controlled complement activation. Functional complement inhibition is not present in the setting of some CFH mutations (Sjoberg, 2009). The interaction of CFH and other complement factors with other loci identified in the genetic predisposition to AMD, such as LOC/ARMS2, remains unknown. The specific systemic and local triggers for complement activation, drusen formation, and progression of AMD remain investigative.

### Macrophages' Role in AMD.

Macrophages and similarly related giant cells have been demonstrated in histologic specimens from patients with AMD, especially in regions of RPE atrophy, breakdown of Bruch's membrane, and choroidal neovascularization. Studies of Ccl2 and Ccr2 knockout in mice, however, suggest a paradoxical role—that macrophages may serve a homeostatic, beneficial role in the retina. While it is generally accepted that macrophages accumulate in the retinal lesions of AMD, it remains unclear whether these macrophages serve a direct role in CNV development or whether they accumulate as an adaptive response to pathological deposits and tissue injury associated with AMD. Recent studies of drusen-like lesions in Ccl2 knockout mice reveal the presence of macrophages, swollen with pigment granules and phagosomes with outer segment and lipofuscin inclusions, in the subretinal space which further corresponded to increased autofluorescence on scanning laser ophthalmoscopy (Luhmann, 2009). The laser-induced CNV studies in these same mice demonstrated that macrophages play a role in the development of CNV, as there was a reduced susceptibility to laser-induced CNV in Ccl2−/− mice (Luhmann, 2009; Tsutsumi, 2003). Indeed, these seemingly paradoxical findings in Ccr2- or Ccl2-deficient mice with laser-induced CNV compared to CNV associated with senescence highlights the potentially dissimilar mechanisms underlying laser-induced CNV and CNV typically associated with age-related maculopathy. It seems likely that laser-induced CNV is a response to acute injury, mediated in part by a sufficient inflammatory response. As such, quelling inflammatory cell recruitment by Ccr2 or Ccl2 deficiency might decrease CNV induced by laser injury. In contrast, in the absence of laser injury, Ccr2 or Ccl2 deficiency may lead to impaired macrophage-mediated clearance of drusen deposits, creating a nidus for low-grade inflammation. This inflammation, over time, may recruit other inflammatory cells, including macrophages and nonmacrophages, potentiating tissue damage and promoting CNV development.

### Significance.

Drusen formation, complement activation, RPE cell dysfunction, local inflammation, and macrophage dysfunction are major mechanisms directly correlated to the development and progression of AMD, but the molecular mechanisms behind their biological interplay are still largely investigative. Previous work implicates RAGE and its ligands as pathway capable of priming the progression of AMD in the aging macula. These observations, and our recent identification of RAGE as a C1q receptor capable of activating complement system, support our hypothesis that in the aging macula, the native biologic functions of RAGE and C1q may be perturbed, particularly in the settings of smoldering accumulation of other ligands for RAGE and of insufficient immune regulation as seen in CFH mutations, to generate and/or sustain local inflammation and cellular damage. Our studies will investigate possible crosstalk between these pathways at a molecular level, specifically characterizing the role of RAGE and its ligands in the binding and activation of C1q and other complement factors and assessing cellular responses and immune mechanisms in this milieu.

### Example 5

### Synopsis of Example 5

Here the biological role of RAGE is elucidated as a C1q binding membrane protein and in its capacity to activate complement and in the modulation of complement-enhanced phagocytosis, complement-dependent cytotoxicity (CDC), and complement-dependent cellular cytotoxicity (CDCC) and to identify a pathogentic role of RAGE and its ligands in cellular dysfunction in AMD in this manner. The identification of inflammatory pathways in the initiations and/or progression of the disorder is important in understanding AMD. Preliminary results demonstrate that RAGE interacts with C1q and can activate the classical complement system. Immune regulatory mechanisms are considered herein as many endogenous C1q-proteins have been found to be able to interact with complement inhibitors. (Sjoberg, 2009)

### (1) Characterization of RAGE/C1q Interaction at the Molecular Level

RAGE functions in a complex structure for its C1q binding related activities. This is based on two lines of supporting evidence. First, similar studies of the interaction of IgG and C1q shows no apparent binding between these proteins unless IgG is part of an immune complex. Second, it has been demonstrated that RAGE ligands induce RAGE oligomerization to activate RPE cells. (Ma, 2007) It is likely that clustered RAGE proteins are also capable of activating the complement system. It is well-known that many proteins implicated in the pathogenesis of AMD are ligands for RAGE, including AGES, amyloid, S100 proteins and CD11b. In this regard, it can be tested to see if RAGE can link the accumulation of these ligands with complement activation. Preliminary data in the aging retina shows distinct populations of RPE cells with regard to SI00b expression within the plasma membrane, suggesting that some cells are targets for RAGE-mediated mechanisms. As understood, complement activation also contains regulatory mechanisms; thus RAGE or its complex structure can also interact with immune regulatory proteins to modulate complement activation when RAGE executes its designated functions in its interactions with C1q.

### (1a). Interplay Between Ligand Binding and C1q Binding on the RAGE Molecule.

RAGE is a multiligand binding protein. Though the exact binding sites are still unknown for all of its ligands, hydrophobic interaction plays an important role for some of its ligands, especially AGEs. As a new member of RAGE binding protein family, C1q has a candidate binding site located at the beginning of RAGE N-terminal V-domain, and this binding is ionic strength dependent indicating its hydrophilic nature. Thus it is likely that the C1q binding site and other ligand binding sites are located at different regions of RAGE and that a complex structure can be formed among RAGE, its ligands and C1q. In this regard, RAGE is an important bridging molecule that connects several proteins to complement activation.

A three components' system, C1q, sRAGE, and one of RAGE's other ligands, are analyzed against two components' systems for interaction studies. Preliminary results demonstrate that pull-down experiments are suitable and thus this technique is being used here. One component is immobilized for pulling down others. When there is complex formation, the two prey proteins will both show up in the pull-down sample with most likely enhanced signal intensity. When two proteins compete for the same binding site on the third protein or there is steric hindrance for their simultaneous binding to the third protein, then at least one prey protein will have reduced signal or no signal at all. In this case, it is impossible to form a complex. Table 3 summarizes experimental outcomes indicative of positive complex formation. Importantly, changes in signal strength from pull down proteins also determine whether there is coordinated binding.

Table 3. Results Showing Positive Among C1q, sRAGE and RAGE's Ligand.

| Immobilized | Pull down | Signal Strength |
|---|---|---|
| Protein | Proteins | (vs. two components system) |
| sRAGE | C1q and Ligand | No change or both stronger |
| C1q | sRAGE and Ligand | Ligand as new signal or intensified |
| Ligand | sRAGE and C1q | C1q as new signal or intensified |

### Experimental Methods:

Human sRAGE is prepared, purified, characterized, and rendered devoid of detectable endotoxin as previously described. (Park, 1998) ELISA-like and Western blot-like assays for this study, as well as traditional pull-down methods. To increase sRAGE and C1q interaction, the ionic strength of the binding buffer is decreased to ½ or ¼ of regular PBS.

### Results and Outcomes

The putative C1q binding site on RAGE is very close to its N-terminal end, it is unlikely to also have all of its other ligand binding sites in this short region given the structural and biochemical differences among these proteins. Studies on S100B binding sites on RAGE demonstrate that RAGE C1 domain is also involved in S100B binding to its V domain, suggesting that these domains form an integrated structural unit for some ligand binding properties. (Dattilo, 2007) Using site-specific antibodies, antibody targeting of the V domain of RAGE attenuates Ap oligomer-induced toxicity in both SHSY-5Y cells and rat cortical neurons, whereas inhibition of Ap aggregate-induced apoptosis requires the neutralization of the C1 domain of the receptor. (Sturchler, 2008) We do not anticipate technical difficulties with these studies but will need to repeat them to confirm results and anticipate future directions. We have demonstrated in our preliminary studies that at least Mac-1, RAGE and C1q are capable of forming a complex structure. We therefore predict that several RAGE ligands may co-bind to RAGE with C1q.

### Alternative Approaches

Although the preliminary data with Mac-1 is consistent with our hypothesis that multiligand RAGE can form a higher-order complex with C1q and RAGE ligands, it is possible that this finding is confined to Mac-1 given the preferential interaction of these ligands with individual domains. In some cases the interaction may be relatively weak for direct binding studies, and it is thus equally important to study complement activation that can also indirectly indicate the existence of such interactions, as is described below in section (1b). Ultimately, it is important to characterize the domain of RAGE that binds C1q.

1(b) Identification of RAGE Ligands and their Physiochemical State(s) that Induce RAGE Molecules to Activate the Complement System

RAGE, like IgG molecules, needs to form oligomers in order to activate the complement system. It has been demonstrated that ligands for RAGE form oligomers under certain conditions, and these ligands further induce RAGE oligomerization to initiate RAGE-dependent signaling cascades in RPE cells. (Ma, 2007) Protein modifications with formation of AGEs can result in multiple modifications upon a single protein molecule and may be enhanced with oxidative stress. Amyloid can form oligomers, aggregates, and fibrils. As for S100B, its special oligomer capacity has been described. (Ma, 2007) The results of these studies strongly suggest that RAGE ligands may induce the oligomerization of RAGE and that ligand and receptor oligomerization serve as an important physiologic threshold for cellular perturbations. Here we determine whether this mechanism can initiate or amplify complement activation. Although weak binding may not be easily assessed with pull-down experiments as described in section 1a above, complement activation indirectly indicates the existence of these bindings. Further, immobilized RAGE ligands or aggregated RAGE ligands may not individually activate serum complement systems, but the addition of sRAGE in serum may result in effective complement activation by ligand-induced oligomerization of ° RAGE molecules.

Here, immobilized RAGE ligand as well as the soluble ligand in liquid phase that have different physicochemical states will be used to determine if RAGE ligands can induce sRAGE oligomerization and result in complement activation.

### Experimental Methods:

AGE-modified BSA (AGE-BSA) is prepared by incubating bovine serum albumin with 200 mmol/L glucose-6-phosphate (G6P) at 37° C. for 8 weeks in 100 mmol/L phosphate (pH 7.4) and 0.5 mmol/L sodium azide. These studies are repeated at concentrations down to 25 mM to better mimic physiologic conditions. At the end of incubation, AGE-BSA preparations are dialyzed against physiological saline. Alternatively, glycated serum albumin can be purchased from Sigma. Oxidized S100B is generated with copper-catalyzed autoxidation of its sulfhydryl groups. (Matsui, 2000) Amyloid oligomer is prepared as previously described. (Ma, 2007) Characterization of the different physicochemical states of RAGE ligands is accomplished by gel electrophoresis and size-exclusion chromatography. (Ma, 2007) Different concentrations of both sRAGE and RAGE ligand are incubated in fresh human serum (Quidel, San Diego, Calif.), and C3a levels in serum are monitored for complement activation.

### Results and Outcomes

Aβ fibrils have been reported to be able to bind C1q and activate the classical complement pathway, (Tsutsumi, 2003) while soluble Ap was not active. (Tacnet-Delorme) In terms of C1 activation, Aβ fibrils appear significantly less efficient than immune complexes. It is important to assess complement activation when Aβ and sRAGE are mixed together in a liquid phase. AGE-BSA may be a more effective molecule for inducing RAGE oligomerization via ligation of RAGE with multiple AGEs on a single BSA. Naturally occurring S100B is a dimer with two RAGE binding sites. Whether this natural form of S100B can induce RAGE dimerization and subsequent activate complement remains to be determined. It will be especially interesting to determine if S100B-induced complement activation via RAGE increases after its oxidation into higher order oligomers.

### Alternative Approaches

By inferring binding interaction by indirect studies of complement activation it may difficult to identify subtle differences in the oligomerization states of RAGE ligands that enhance C1q binding and complement activation. Still, the triggers for complement activation in the aging macula are unknown, and the concept of oligomerization of ligands as resulting in a threshold for effective activation make it important characterize the most effective states of RAGE ligands for multivalent C1q binding with RAGE. More direct studies of binding, after the ligand/physiochemical states are identified, will be helpful in understanding the domain of RAGE that binds C1q itself.

### 1(c) Immune Regulatory Mechanisms on RAGE/C1q Interaction

The complement system was initially recognized as a defense system against infections; however, it is now clear that complement also functions as an important humoral system to sense danger including damaged or altered self tissues. This complex pattern of responses enables the body to react in a different manner according to the various types of danger, and it must be closely regulated to prevent immunopathology. For some types of danger signals, the complement system initiates a strong inflammatory response, whereas for others it merely flags the target molecules and/or cells for enhanced phagocytosis. The complement pathway is an aggressive proteolytic cascade working under the tight control of inhibitors, and its final effects with each trigger depend upon the biologic balance between activation and inhibition. Unlike immune complexes, many endogenous targets that are recognized by C1q and initiate the classical pathway also interact with the complement inhibitors factor H (CFH) and C4b-binding protein (C4BP). Thus some endogenous targets can activate the early classical complement pathway as efficiently as immune complex but the terminal complement pathway is poorly triggered, with examples including fibromodulin, (Barile, 2005) osteoadherin, (Sjoberg, 2009) C-reactive protein, (Jarva, 1999) and amyloid. (Trouw, 2008) All physiologic effects of the observed CFH polymorphisms in AMD are not yet fully elucidated from protein function studies, but the polymorphism has been shown to alter the interaction between CFH and heparin, glycosaminoglycans, CRP, fibromodulin and chondroadherin, DNA and necrotic cells. (Herbert, 2007; Laine, 2007; Sjoberg, 2009; Sjoberg, 2007) The current hypothesis for the involvement of CFH in AMD is that the risk-associated allele has an impaired ability to inhibit complement activation, leading to excessive inflammation in the aging macula. In this regard, we will examine complement regulation on RAGE/C1q interaction.

Originally, CFH was considered a central regulator of the alternative pathway in the fluid phase and upon cell surfaces, where it achieves complement homeostasis through the restriction of excessive alternative pathway activation. Later studies of its mechanism revealed that CFH limits complement activation through the inactivation of deposited C3b on self-cells after complement activation through any of the three initiation pathways. (Boon, 2009; Suankratay, 1998) In mice, the classical complement pathway activation by immune complexes can recruit the alternative pathway if the alternative pathway is not effectively regulated by CFH. (Alexander, 2003) Through these mechanisms, CFH exerts a potent inhibitory influence on the activity of the common complement pathways.

### Experimental Methods

### Direct Binding Tests:

Endogenous C1q binding proteins normally cannot activate the complement system unless they are clustered, polymerized, or immobilized, in which cases C1q has a stronger binding affinity to these proteins. (Yin, 2007) A similar principle may apply to complement inhibitors, and thus immobilized sRAGE is used to pull down CFH and C4BP. It is also possible that CFH and C4BP may not bind directly to RAGE but to its ligand that can form an active complex with RAGE; for example, in the case of RAGE and Mac-1, preliminary results show that they can form an effective complex for complement activation, and Mac-1 has also been reported to be able to bind CFH. (DiScipio, 1998) Thus, both RAGE ligands and RAGE-ligand complexes are tested.

### Complement Inhibition Tests:

Depletion of CFH or C4BP from human serum are completed by passing the serum through a column of CFH or C4BP antibody coupled to CNBr-activated Sepharose, respectively. CFH and C4BP depletion is confirmed by Western blot. Complement activation by RAGE or RAGE-containing complexes is compared between native human serum and component-depleted serum. If depletion of CFH or C4BP enhances the formation of C3a, it indicates that CFH or C4BP is a regulator for this process.

### Results and Outcomes

RAGE has two N-glycosylation sites in its ligand-binding V-domain, and analysis of glycosaminoglycans from lung RAGE indicates that they are negatively-charged glycans. (Srikrishna, 2002) These glycans may have binding affinity on the SCR20 and SCR7 domains of CFH. In the case of a RAGE-Mac-1 complex, CFH binding may be enhanced by binding to both Mac-1 and RAGE. If positive binding of CFH to sRAGE is observed the binding study is repeated with risk-associated CFH to determine if there is difference.

### Alternative Approaches

Alternative approaches to identifying, directly and indirectly, evidence of CFH and/or C4BP interaction with RAGE, when this interaction may only occur physiologically in higher-order active complex formation of RAGE with its ligands, are studies of native complement-dependent cytotoxicity which are discussed herein in section which are performed in the absence of CFH.

### Sequence Analysis

After the characterization of the RAGE/C1q interaction at the molecular level, C1q binding site on RAGE is analyzed, sequence analysis is used to pinpoint an IgG-like C1q-binding site near N-terminal of RAGE molecule (see preliminary data); sequence deletion and mutation at this possible binding site is first tested to determine that it contains the binding site for C1q. It is well known that the core C1q binding site on IgG molecules is ExKxK, and that replacement of amino acid E with V reduces its binding affinity to one tenth. (Duncan, 1988) RAGE has a sequence of VLKCK near its N-terminal and also has a roughly one tenth binding affinity for C1q (preliminary data FIG. 10F) and one eighth complement activation activity of human IgG (preliminary data FIG. 1). Amino acid V in this possible C1q binding sequence is mutated back to E and the mutated RAGE is studied for its binding affinity for C1q and complement activation activity. In addition, V-domain of RAGE is deleted to further confirm whether the C1q binding site is located in this domain. Finally, a general strategy based upon the overall structure of RAGE is utilized to locate its C1q binding domain in C1 and C2. (Leclerc 2009).

### (2) RAGE/C1q Interaction at the Cellular Level

RAGE-bearing cells will only activate complement system when RAGE forms an oligomer and/or a complex with integrins or other RAGE ligands along the cellular membrane. As RAGE is a membrane protein that has restricted movement on the cell surface, it is necessary to test whether RAGE expressing cells can bind C1q and activate the complement system. Two approaches are, first employing overexpression of RAGE in RPE cells (which normally do not express beta2 integrins such as Mac-1). This study determines whether C1q can stably attach to RPE cells via multiple binding to cellular RAGE receptors. The effect of extracellular addition of RAGE ligands such as Mac-1 is also studied. The second approach compares C1q binding and complement activation between normal monocytes and RAGE eliminated monocytes. It is known that both RAGE and Mac-1 are present in monocytes/macrophages; thus a complex formation of RAGE and Mac-1 on the plasma membrane of theses cells is expected. The second approach is augmented by experiments described in section 3 which focus on the RAGE knockout effect upon complement-related phagocytosis and cytotoxicity.

### Experimental Results

As RAGE expression in RPE cells is normally low, RAGE expression in human RPE cells is manipulated (cell line ARPE-19) to facilitate the research and mimic pathological conditions with gene transfection techniques. RPE cell lines bearing either full length RAGE gene or dominant negative RAGE (lacking the cytosolic tail of full length RAGE) gene are generated with pcDNA3 plasmid vector (Taguchi, 2000) and transfection reagent (Lipofectamine 2000; Invitrogen) according to previously reported methods. (Ma, 2007) Cells are selected in the presence of geneticin (G418), 1.0 mg/ml, and individual clones will be isolated by limiting dilution. C1q binding is tested in serum-free medium by monitoring C1q level changes after one hour incubation with confluent RPE cells. The effect of RAGE ligands is also be included. A sensitive C1q ELISA that detects ng/ml of C1q has been established. For complement activation studies, human serum is used for incubation with a monolayer of RPE cells, and the C3a level is monitored. Also, immunostaining of RPE cells for C3c is performed to confirm serum C3a results. The methods regarding isolation and preparation of RAGE-bearing and RAGE-knockout monocytesare disclosed elsewhere in this application, for example see section 3(a) herein.

### Results and Outcomes

Based on the biochemical properties of RAGE with each molecule having a strong acidic tail, it is predicted that RAGE alone cannot form cellular oligomers to stably bind C1q for complement activation. In the case of RAGE-Mac-1 complex on monocytes/macrophages, C1q binding likely is stronger with effective complement activation occurring under certain conditions such as the presence of oligomeric RAGE ligands. Such outcomes suggest that only local macrophages are required to mediate cell-induced complement activation via RAGE-C1q interaction in the outer retina.

### Alternative Approaches

If it is observed that native or RAGE overexpressing RPE cells are capable of activating complement under certain conditions, these studies are repeated with RAGE knockout cells to confirm RAGE-specificity. It is known that accumulation of A2E and other metabolic products are also capable of triggering complement activation, and assessment of any such contribution is important to understanding a surprising result.

### (3) Determination of the Role of RAGE/C1q Interaction on Complement-Enhanced Phagocytosis, Complement-Dependent Cytotoxicity (CDC) and Complement-Dependent Cellular Cytotoxicity (CDCC)

The homeostatic biological function of RAGE and C1q interaction includes leukocyte recruitment and apoptotic cell clearance but under certain conditions this process may generate unintended effects of complement activation, immunopathology, and cellular damage. In monocytes, RAGE plays an essential role for proper phagocytosis as a C1q receptor. In the setting of defects in complement inhibitory mechanisms, there is enhanced CDC upon monocytes/macrophages via RAGE's role as a C1q receptor that also interacts with Mac-1.

### 3(a) Effect of RAGE Knockout on Complement Enhanced Phagocytosis

The most characterized immunological function of C1q is as recognition component for the activation of the classical complement pathway. However, it is known that C1q is synthesized and secreted in the absence of C1r and C1s. In addition, C1q is found in vivo in the absence of C1r and C1s, and thus additional roles for C1q are plausible. C1q receptors are involved in the diverse functions of C1q, and C1q enhanced phagocytosis may be a major biological process among them.

### Experimental Methods

Homozygous RAGE knockout mice provide a key method to test the impact of deletion of RAGE on complement enhanced phagocytosis. RAGE knockout mice have been backcrossed 10 generations into the C57BL/6 background; homozygous RAGE knockout are generated by fresh breeding of RAGE+/− with RAGE+/− without inbreeding Blood is collected at age of about 16 weeks. Isolation of neutrophils and monocytes is performed with Histopaque 1077 and Histopaque 1119 (Sigma) per manufacturer's instructions. Coating of red-fluorescent latex beads with C1q is performed according to Sigma's instructions. Monocytes are washed and resuspended with C1q-coated beads at a ratio of 1:10 in phagocytosis buffer. After incubation at 37° C. for 30 minutes, cells are washed two times in phagocytosis buffer and phagocytic particle uptake is calculated from data obtained by microscopy and flow cytometry. Cells incubated in buffer alone are included as a negative control.

### Results and Outcomes

RAGE-eliminated monocytes displaying weakened phagocytic ability for C1q coated beads suggest that RAGE plays an essential role for proper phagocytosis as a C1q receptor. This finding supports the notion of an important native, homeostatic biological function for RAGE and C1q interaction. It would be surprising if the RAGE-eliminated monocytes do not demonstrate decreased C1q-mediated phagocytic ability.

### (3b) Effect of RAGE Knockout on CDC

Cellular death due to necrosis usually does not emit the chemical signals to the immune system that cellular apoptosis does. This may prevent nearby phagocytes from locating and engulfing the dead cells, leading to an accumulation of dead tissue and cellular debris at or near the site of the cell death. It is well known that the presence of larger and more numerous drusen in the macula is a common early sign of AMD, but the earliest cellular events involved in drusen formation remain unknown despite their longstanding recognition and description. Senescent Ccl2- or Ccr2-deficient mice develop AMD-like pathology including accumulation of lipofuscin in RPE, drusen formation, photoreceptor atrophy and choroidal neovascularization. (Ambati, 2003) Impaired macrophage function may contribute to AMD pathology in this model because swollen macrophages were found to have accelerated accumulation in the subretinal space. (Ambati, 2003; Luhmann, 2009) RAGE and Mac-1 expressing monocytes/macrophages are investigated for C1q binding and complement activation. With the aid of RAGE elimination from monocytes/macrophages and CFH depletion from normal serum, it can be tested whether defects in immune regulatory mechanisms generate damage upon monocytes/macrophages through RAGE-induced complement activation. Another possible scenario is that RAGE-expressing RPE cells interact with macrophages through binding of RAGE to Mac-1; C1q may enhance this process while also causing complement activation with this interaction.

### Experimental Methods

Monocytes obtained in section (3a) above are utilized for C1q binding and complement activation tests using similar methods described section (2) above. Depletion of C1q or CFH from mouse serum is completed by a similar procedure described in section (1c) by using anti-mouse antibodies. Sera with full complement system or component-depleted is used to test CDC effect on normal and RAGE-eliminated monocytes. In addition, C1q-coated latex beads are added into the sera to determine if CDC is mechanism-dependent. The human monocyte/macrophage cell lines THP-1 and U937 (American Type Culture Collection, Manassas, Va.) are used for cell co-culture studies. Cells are grown in suspension in RPMI medium. The human ARPE-19 and RAGE over-expressing ARPE-19 cells are grown in DMEM medium. In co-culture experiments, all cells are incubated in human serum. THP-1 and U937 cells are added into RPE cell culture dishes and incubated for different times with human serum containing full complement system or component-depleted system. Complement activation is monitored at different stages of the experiment. Early C3a level in serum indicates whether complement system is activated; immunohistochemical stains of monocytes and RPE cells for C5-C9 complex indicate whether complement activation proceeds to the final step of the cascade. Changes in monocytes number and RPE cell number are the final indicators assessing whether RAGE-induced CDC is strong enough to generate biologically significant damage on monocytes or RPE cells.

### Results and Outcomes

RAGE can activate the complement system, but its activity is much weaker in comparison to the native immune complex. RAGE-bearing monocytes likely display mechanism-dependent CDC, while RAGE eliminated monocytes are likely relatively resistant to CDC. When complement inhibition is deficient (CFH absence), however, complement-dependent cytotoxicity is likely enhanced in RAGE bearing monocytes and RPE cells. Such an outcome improves the understanding of the effect of CFH mutations in the pathogenesis of AMD and specifically suggests a predilection for CDC in RAGE-bearing cells in the outer retina.

### Alternative Approaches

These experiments may not demonstrate an absolute effect of RAGE elimination, again because of the possibility of alternative C1q receptors than RAGE. There may, instead, be a relative deficiency in CDC that is more difficult to quantify. The goals of these studies are to identify the native role of RAGE as a C1q receptor. An alternative approach focuses upon the deleterious effects of sRAGE activation of complement which is known to occur from preliminary studies.

### (3c) Effect of RAGE and CDCC

RAGE activation of NADPH oxidase is well-documented. (Alexander, 2007; Warboys, 2009; Wautier, 2001) It has also been demonstrated that immobilized C1q can trigger a respiratory burst by an unique CD18-dependent mechanism. (Goodman, 1995; Tenner, 1982; Tyagi, 2000) But the (32 integrin alone has not been successfully demonstrated to serve as C1q receptor, instead providing an essential link between C1q receptor ligation and NADPH oxidase activation. The RAGE-Mac-1 complex is a candidate receptor complex that can combine C1q binding and cellular activation together. Since the identification of RAGE and Mac-1 interaction, it has been demonstrated that RAGE and Mac-1 can be located on the same cell or on different cells. (Chavakis, 2003; Orlova, 2007) RAGE-expressing endothelial cells can recruit Mac-1 expressing leukocytes by a direct interaction of RAGE with the beta2-integrin Mac-1, which can be augmented by the proinflammatory RAGE-ligand, S100-protein. (Chavakis 2003) Studies also found that Mac-1-dependent neutrophil recruitment induced by HMGB1 required the presence of RAGE on neutrophils but not on endothelial cells. (Orlova, 2007) In that report, HMGB1 enhanced the interaction between Mac-1 and RAGE.

### Experimental Methods

Monocytes obtained in section 3a above are used for CDCC studies. Monocytes are activated by C1q coated on cluster plate or on latex beads, and the production of superoxide anion is monitored by the superoxide dismutase sensitive reduction of ferricytochrome c, which is recorded at 550 nm against time using a plate reader. Cytokines and chemokines levels released by monocytes, such as IL-1, IL-6, INF-γ, VEGF, are also be monitored by ELISA. The human monocyte/macrophage cell lines THP-1 and U937 used in section 3b above are also be used for cell co-culture studies. A mixture medium (1:1) of RPMI and DMEM is used in co-culture experiments. To visualize the THP-1 and U937 cells adherence to the monolayer of RPE cells, the THP-1 and U937 cells are labeled with a fluorescent dye, calcein-AM, by pre-loading the cells with 10 μM of calcein-AM at 37° C. for 1 hour. Calcein-AM-labeled THP-1 and U937 cells are added into RPE cell culture dishes and incubated for different times. After removing of non-adherent cells by gentle washing, the number of adherent THP-1 and U937 cells is determined by measuring fluorescence intensity of the adherent cells using a fluorometer and also by counting the number of adherent fluorescence-labeled cells with a fluorescence microscope. Effects of the addition of human C1q and blocking antibodies of RAGE, CD 18, and CD11b on the adhesion is also studied. Co-culture experiments are also be performed in a trans-well system where the cells are separated by a porous membrane; RPE cells will grow in the lower compartment and THP-1 or U937 cells are incubated in the upper compartment. THP-1 or U937 cells are activated by C1q coated latex beads and cellular mediators released from THP-1 or U937 cells can then reach RPE cells. Cytotoxicity is examined using a MTT-based assay and by measuring lactate dehydrogenase-leakage. Real Time PCR is utilized for expression analysis of genes such as VEGF, PEDF, PDGF, bFGF, TGF-β, IGF-1, ICAM, etc.

### Results and Outcomes

If the RAGE and Mac-1 complex in monocytes is involved in phagocytosis of C1q-opsonized targets (Section 3a), RAGE and Mac-1 would be expected to contribute to the CDCC that accompanies phagocytosis. RAGE-eliminated monocytes will likely be relatively deficient in CDCC. The interactions between monocytes and RPE cells may suggest a role for RAGE and Mac-1 in native adhesion, with the effects of C1q further enhancing this interaction.

### (4) Characterizing the Presence and Distribution of RAGE, its Lagands and Complement Components as Well as Macrophages in Aged Monkey Retina, an Early AMD Model

RAGE expressing cells in retina contribute differently to the development of AMD, depending upon cell type, location and life duration. RPE cells adjacent to Bruch's membrane deposits and drusen express RAGE and SI00b, which suggests that these RPE cells are potential targets for complement attack. Macrophages may have different actions when they cross the outer blood retinal barrier layer. Outside of this layer, macrophages or macrophage-RPE are exposed to the complement system. If complement regulatory mechanisms cannot effectively inhibit complement activation, macrophages may not remain sufficiently healthy or durable to clear cellular debris and instead dead macrophages may contribute to deposits in the outer retina. Inside the RPE layer, macrophages may function mainly as housekeeping cells including clearance of C1q tagged cells, which themselves may be overactivated in neurodegenerative disease.

A rhesus monkey model of drusenoid maculopathy (Gouras, 2008; Gu, 2008) is used. Antibodies of RAGE, AGEs, 5100 proteins, HMGB1, amyloid, complement proteins, complement inhibitors, and several cellular markers are employed to examine the existence and distribution of these proteins in the older monkey retina that displays early signs of AMD. A major reason for choosing this monkey model is that, unlike other primate models, this monkey model develops truly human-like early AMD with classic macular drusen. Any changes that are detected with these antibodies are likely relevant to human changes.

### Experimental Methods

Eyes from 1-5 and 20-35 years old rhesus monkeys with and without moderately severe drusenoid maculopathy have been fixed in 4% paraformaldehyde in phosphate buffered saline (PBS), with the globes are pierced to facilitate diffusion of the fixative into the vitreous (Available from the laboratory of Dr. Gouras). After storage at 4° C. in fixative, the eyes are washed with PBS and dissected with the aid of a surgical microscope. The macula is identified and cut into a square approximately 15×15 mm, centered on the fovea. Similar segments will be obtained from the equatorial region for comparison with macula areas. Immunohistochemistry or immunofluorescence combined with confocal laser scanning light microscopy is used for this study as performed in previous studies. (Barile, 2007). In addition immunogold techniques are used to locate what cellular structures are immunolabeled. In this case pre-embedding immunolabeling is performed. The retina and choroid, fixed by paraformaldehyde, are cryosectioned and then exposed to primary and secondary antibodies. Then the sections mounted on a glass slide are dehydrated and embedded in epon. The slide and epon embedded sections are heated to 90° C. and the epon segment separated from the glass slide with a razor blade. The epon segment containing the retina and choroid is glued to the cutting block and thick and thin sections cut and examined by light and electron microscopy. Ultra-thin sections are examined by transmission electron microscopy using Zeiss IOC or Jeol 1200 EX2 instruments. In some cases serial sectioning will be performed. Digital photographs are examined and processed by Adobe Photoshop.

### Results and Outcomes

The most likely RAGE ligands that are detected at the RPE cell layer of eyes with AMD are S100 proteins and amyloid oligomers. These proteins may be generated locally by RPE cells and further retained at the plasma membrane by binding their receptors. AGEs will likely be detected at within Bruch's membrane and drusen deposits.

### Statistical Analysis

Where needed, power analyses are applied to provide the number of animals required. Significant differences between experimental groups (time points, genotypes, conditions) are detected using analysis of variance for unpaired observations or student's T-test for basic two groups' analysis. Posthoc comparisons are performed using mostly Tukey's procedure. SPSS software is used for statistical analysis (SPSS, Chicago, Ill.).

### REFERENCES

1. Alexander J J and Quigg R J. The simple design of complement factor H: Looks can be deceiving. Molecular immunology 44: 123-132, 2007.
2. Ambati et al. An animal model of age-related macular degeneration in senescent Ccl-2- or Ccr-2-deficient mice. Nature medicine 9: 1390-1397, 2003.
3. Anderson et al. Characterization of beta amyloid assemblies in drusen: the deposits associated with aging and agerelated macular degeneration. Experimental eye research 78: 243-256, 2004.
4. Ballinger et al. Glycated and carboxy-methylated proteins do not directly activate human vascular smooth muscle cells, Kidney Int. 68 (2005) 2756-2765.
5. Banerjee et al. The C-terminal acidic tail is responsible for the inhibitory effects of HMGB1 on efferocytosis, J. Leukoc. Biol. 88 (2010) 973-979.
6. Barile et al. The RAGE axis in early diabetic retinopathy. Investigative ophthalmology & visual science 46: 2916-2924, 2005.
7. Barile G R and Schmidt A M. RAGE and its ligands in retinal disease. Current molecular medicine 7: 758-765, 2007.
8. Boon et al. The spectrum of phenotypes caused by variants in the CFH gene. Molecular immunology 46: 1573-1594, 2009.
9. Botto et al. Homozygous C1q deficiency causes glomerulonephritis associated with multiple apoptotic bodies. Nature genetics 19: 56-59, 1998.
10. Buckley, S T. et al. “The receptor for advanced glycation end products and the lung” Journal of biomedicine and Biotechnology 2010. ID917108
11. Bureeva et al. Inhibition of classical pathway of complement activation with negative charged derivatives of bisphenol A and bisphenol disulphates. Bioorganic & Medicinal Chemistry, 2005, 13:1045-1052
12. Chavakis et al. The pattern recognition receptor (RAGE) is a counterreceptor for leukocyte integrins: a novel pathway for inflammatory cell recruitment. The Journal of experimental medicine 198: 1507-1515, 2003.
13. Chen et al. RAGE ligation affects T cell activation and controls T cell differentiation, J. Immunol. 181 (2008) 4272-4278.
14. Dattilo et al. The extracellular region of the receptor for advanced glycation end products is composed of two independent structural units. Biochemistry 46: 6957-6970, 2007.
15. Defacque et al. Commes, Transforming growth factor-beta1 is an autocrine mediator of U937 cell growth arrest and differentiation induced by vitamin D₃ and retinoids, J. Cell. Physiol. 178 (1999) 109-119.
16. Dentchev et al. Amyloid-beta is found in drusen from some age-related macular degeneration retinas, but not in drusen from normal retinas. Molecular vision 9: 184-190, 2003.
17. Ding et al. Activation of RAGE induces elevated O2-generation by mononuclear phagocytes in diabetes. Journal of leukocyte biology 81: 520-527, 2007.
18. DiScipio et al. Human polymorphonuclear leukocytes adhere to complement factor H through an interaction that involves alphaMbeta2 (CD11b/CD18). J Immunol 160: 4057-4066, 1998.
19. Duncan A R and Winter G. The binding site for C1q on IgG. Nature 332: 738-740, 1988.
20. Duus et al. Thielens, G. Houen, Direct interaction between CD91 and C1q, FEES J. 277 (2010) 3526-3537.
21. Ferhani et al. Expression of high-mobility group box 1 and of receptor for advanced glycation end products in chronic obstructive pulmonary disease, Am. J. Respir. Crit. Care Med.
181 (2010) 917-927.
22. Friggeri et al. Participation of the receptor for advanced glycation end products in efferocytosis, J. Immunol. 186 (2011) 6191-6198.
23. Gaboriaud et al. The crystal structure of the globular head of complement protein C1q provides a basis for its versatile recognition properties, J. Biol. Chem. 278 (2003) 46974-46982.
24. Ghiran et al. Expression and function of C1q receptors and C1q binding proteins at the cell surface, Immunobiology 205 (2002) 407-420.
25. Gingras and Margolin. Differential expression of multiple unexpected genes during U937 cell and macrophage differentiation detected by suppressive subtractive hybridization, Exp. Hematol. 28 (2000) 65-76.
26. Glenn et al. Advanced glycation end product (AGE) accumulation on Bruch's membrane: links to age-related RPE dysfunction. Investigative ophthalmology & visual science 50: 441-451, 2009.
27. Goodman et al. C1q triggers neutrophil superoxide production by a unique CD18-dependent mechanism. Journal of leukocyte biology 58: 168-176, 1995.
28. Gouras et al. Drusenoid maculopathy in rhesus monkeys (Macaca mulatta): effects of age and gender. Graefe's archive for clinical and experimental ophthalmology=Albrecht von Graefes Archiv fur klinische and experimentelle Ophthalmologie 246: 1395-1402, 2008.
29. Gu et al. Comparison of Proteins in Dry and Wet AMD Bruch's Membrane Investigative ophthalmology & visual science 50: E-Abstract 2343, 2009.
30. Gu et al. Quantitative Proteomic Analysis of AMD Bruch's Membrane. Investigative ophthalmology & visual science 49: E-Abstract 1753, 2008.
31. Hageman et al. An integrated hypothesis that considers drusen as biomarkers of immune-mediated processes at the RPEBruch's membrane interface in aging and age-related macular degeneration. Progress in retinal and eye research 20: 705-732, 2001.
32. Hammes et al. N(epsilon)(carboxymethyl)lysin and the AGE receptor RAGE colocalize in age-related macular degeneration. Investigative ophthalmology & visual science 40: 1855-1859, 1999.
33. Handa et al. Increase in the advanced glycation end product pentosidine in Bruch's membrane with age. Investigative ophthalmology & visual science 40: 775-779, 1999.
34. He et al. Receptor for advanced glycation end products binds to phosphatidylserine and assists in the clearance of apoptotic cells, EMBO Rep. 12 (2011) 358-364.
35. Herbert et al. Structure shows that a glycosaminoglycan and protein recognition site in factor H is perturbed by age-related macular degeneration-linked single nucleotide polymorphism. The Journal of biological chemistry 282: 18960-18968, 2007.
36. Hori et al. The receptor for advanced glycation end products (RAGE) is a cellular binding site for amphoterin. Mediation of neurite outgrowth and co-expression of rage and amphoterin in the developing nervous system, J. Biol. Chem. 270 (1995) 25752-25761.
37. Howes et al. AMD-Like Retinal Degeneration in S100B Transgenic Mice. Investigative ophthalmology & visual science 43: E-Abstract 2812, 2002.
38. Howes et al. Receptor for advanced glycation end products and age-related macular degeneration. Investigative ophthalmology & visual science 45: 3713-3720, 2004.
39. Idusogie et al. Mapping of the C1q binding site on rituxan, a chimeric antibody with a human IgG1 Fc. J Immunol 164: 4178-4184, 2000.
40. Ishibashi et al. Advanced glycation end products in age-related macular degeneration. Archives of ophthalmology 116: 1629-1632, 1998.
41. Jarva et al. Regulation of complement activation by C-reactive protein: targeting the complement inhibitory activity of factor H by an interaction with short consensus repeat domains 7 and 8-11. J Immunol 163: 3957-3962, 1999.
42. Johnson et al. The Alzheimer's A beta-peptide is deposited at sites of complement activation in pathologic deposits associated with aging and age-related macular degeneration. Proceedings of the National Academy of Sciences of the United States of America 99: 11830-11835, 2002.
43. Kaji et al. Expression and function of receptors for advanced glycation end products in bovine corneal endothelial cells. Investigative ophthalmology & visual science 44: 521-528, 2003.
44. Kaji et al. Inhibition of diabetic leukostasis and blood-retinal barrier breakdown with a soluble form of a receptor for advanced glycation end products. Investigative ophthalmology & visual science 48: 858-865, 2007.
45. Kaul and Loos. Dissection of C1q capability of interacting with IgG. Timedependent formation of a tight and only partly reversible association, J. Biol. Chem. 272 (1997) 33234-33244.
46. Kaul M and Loos M. Dissection of C1q capability of interacting with IgG. Time-dependent formation of a tight and only partly reversible association. The Journal of biological chemistry 272: 33234-33244, 1997.
47. Kishore et al. Structural and functional anatomy of the globular domain of complement protein C1q, Immunol. Lett. 95 (2004) 113-128.
48. Kokkola et al. RAGE is the major receptor for the proinflammatory activity of HMGB1 in rodent macrophages, Scand. J. Immunol. 61 (2005) 1-9.
49. Kolble K and Reid K B. Genetic deficiencies of the complement system and association with disease—early components. International reviews of immunology 10: 17-36, 1993.
50. Laine et al. Y402H polymorphism of complement factor H affects binding affinity to C-reactive protein. J Immunol 178: 3831-3836, 2007.
51. Lauvrak et al. Identification and characterisation of C1q binding phage displayed peptides. Biological chemistry 378: 1509-1519, 1997.
52. Leclerc et al. Binding of 5100 proteins to RAGE: an update. Biochimica et biophysica acta 1793: 993-1007, 2009.
53. Liliensiek et al. Receptor for advanced glycation end products (RAGE) regulates sepsis but not the adaptive immune response, J. Clin. Invest. 113 (2004) 1641-1650.
54. Lin, L. et al. “RAGE signaling in inflammation and arterial aging”. Frontiers in Bioscience 2009. V. 14 p. 1403-1413.
55. Lu et al., The regulatory roles of C1q, Immunobiology 212 (2007) 245-252.
56. Luhmann et al. The drusen-like phenotype in aging Ccl2 knockout mice is caused by an accelerated accumulation of swollen autofluorescent subretinal macrophages. Investigative ophthalmology & visual science, 2009.
57. Ma et al. RAGE ligand upregulation of VEGF secretion in ARPE-19 cells. Investigative ophthalmology & visual science 48: 1355-1361, 2007.
58. Manfredi et al. Maturing dendritic cells depend on RAGE for in vivo homing to lymph nodes, J. Immunol. 180 (2008) 2270-2275.
59. Matsui Lee et al. Copperdependent formation of disulfide-linked dimer of S100B protein. Archives of biochemistry and biophysics 374: 137-141, 2000.
60. McGreal et al. Human C1qRp is identical with CD93 and the mNI-11 antigen but does not bind C1q, J. Immunol. 168 (2002) 5222-5232.
61. Mevorach et al. Complement-dependent clearance of apoptotic cells by human macrophages. The Journal of experimental medicine 188: 2313-2320, 1998.
62. Moser et al. Blockade of RAGE suppresses alloimmune reactions in vitro and delays allograft rejection in murine heart transplantation, Am. J. Transplant. 7 (2007) 293-302.
63. Mullins et al. Drusen associated with aging and agerelated macular degeneration contain proteins common to extracellular deposits associated with atherosclerosis, elastosis, amyloidosis, and dense deposit disease. Faseb J 14: 835-846, 2000.
64. Nayak et al. The non-classical functions of the classical complement pathway recognition subcomponent C1q, Immunol. Lett. 131 (2010) 139-150.
65. Neeper et al. Cloning and expression of a cell surface receptor for advanced glycosylation end products of proteins. The Journal of biological chemistry 267: 14998-15004, 1992.
66. Nimmerjahn and Ravetch., Fcgamma receptors as regulators of immune responses, Nat. Rev. Immunol. 8 (2008) 34-47.
67. Origlia, N et al. “MARK, β-amyloid and synaptic dysfuction: the role of RAGE” Expert Reviews of Neurotherapeutics 2009. V. 9 p. 1635-1645.
68. Orlova et al. A novel pathway of HMGB1-mediated inflammatory cell recruitment that requires Mac-1-integrin. The EMBO journal 26: 1129-1139, 2007.
69. Pachydaki et al. Upregulation of RAGE and its ligands in proliferative retinal disease. Experimental eye research 82: 807-815, 2006.
70. Paidassi et al. C1q binds phosphatidylserine and likely acts as a multiligand-bridging molecule in apoptotic cell recognition, J. Immunol. 180 (2008) 2329-2338.
71. Park et al. Suppression of accelerated diabetic atherosclerosis by the soluble receptor for advanced glycation endproducts. Nature medicine 4: 1025-1031, 1998.
72. Perry et al. C1q: the perfect complement for a synaptic feast?, Nat Rev. 9 (2008) 807-811.
73. Ramasamy et al. Advanced glycation end products and RAGE: a common thread in aging, diabetes, neurodegeneration, and inflammation. Glycobiology 15: 16R-28R, 2005.
74. Ramasamy et al. RAGE: therapeutic target and biomarker of the inflammatory response—the evidence mounts. Journal of leukocyte biology 86: 505-512, 2009.
75. Reiniger et al. Deletion of the receptor for advanced glycation end products reduces glomerulosclerosis and preserves renal function in the diabetic OVE26 mouse, Diabetes 59 (2010) 2043-2054.
76. Ruan et al. Complement C3a, CpG oligos, and DNA/C3a complex stimulate IFN-alpha production in a receptor for advanced glycation end product-dependentmanner, J. Immunol. 185 (2010) 4213-4222.
77. Schmidt et al., Receptor for age (RAGE) is a gene within the major histocompatibility class III region: implications for host response mechanisms in homeostasis and chronic disease. Front Biosci. (2001) 6:D1151-60.
78. Seth et al. Complement-associated deposits in the human retina. Investigative ophthalmology & visual science 49: 743-750, 2008.
79. Sjoberg et al. Complement activation and inhibition: a delicate balance. Trends in immunology 30: 83-90, 2009.
80. Sjoberg et al. The factor H variant associated with age-related macular degeneration (His-384) and the non-diseaseassociated form bind differentially to C-reactive protein, fibromodulin, DNA, and necrotic cells. The Journal of biological chemistry 282: 10894-10900, 2007.
81. Soulis et al. Advanced glycation end products and their receptors co-localise in rat organs susceptible to diabetic microvascular injury. Diabetologia 40: 619-628, 1997.
82. Srikrishna et al. N-Glycans on the receptor for advanced glycation end products influence amphoterin binding and neurite outgrowth. Journal of neurochemistry 80: 998-1008, 2002.
83. Stevens et al. The classical complement cascade mediates CNS synapse elimination. Cell 131: 1164-1178, 2007.
84. Sturchler et al. Site-specific blockade of RAGE-Vd prevents amyloid-beta oligomer neurotoxicity. J Neurosci 28: 5149-5158, 2008.
85. Suankratay et al. Requirement for the alternative pathway as well as C4 and C2 in complement-dependent hemolysis via the lectin pathway. J Immunol 160: 3006-3013, 1998.
86. Tacnet-Delorme et al. Beta-amyloid fibrils activate the C1 complex of complement under physiological conditions: evidence for a binding site for A beta on the C1q globular regions. J Immunol 167: 6374-6381, 2001.
87. Taguchi et al. Blockade of RAGE-amphoterin signaling suppresses tumour growth and metastases, Nature 405 (2000) 354-360.
88. Taguchi et al. Blockade of RAGE-amphoterin signalling suppresses tumour growth and metastases. Nature 405: 354-360, 2000.
89. Teh et al. C1q regulation of dendritic cell development from monocytes with distinct cytokine production and T cell stimulation, Mol. Immunol. 48 (2011) 1128-1138.
90. Tenner A J and Cooper N R. Stimulation of a human polymorphonuclear leukocyte oxidative response by the C1q subunit of the first complement component. J Immunol 128: 2547-2552, 1982.
91. Trouw et al. C4b-binding protein in Alzheimer's disease: binding to Abeta1-42 and to dead cells. Molecular immunology 45: 3649-3660, 2008.
92. Tsutsumi C, Sonoda K H, Egashira K, Qiao H, Hisatomi T, Nakao S, Ishibashi M, Charo I F, Sakamoto T, Murata T, and Ishibashi T. The critical role of ocular-infiltrating macrophages in the development of choroidal neovascularization. Journal of leukocyte biology 74: 25-32, 2003.
93. Tyagi et al. Intercellular adhesion molecule 1 and beta2 integrins in C1q-stimulated superoxide production by human neutrophils: an example of a general regulatory mechanism governing acute inflammation. Arthritis and rheumatism 43: 2248-2259, 2000.
94. van der Schaft et al. Early stages of age-related macular degeneration: an immunofluorescence and electron microscopy study. The British journal of ophthalmology 77: 657-661, 1993.
95. van Zoelen et al. Ligands of the receptor for advanced glycation end products, including high-mobility group box 1, limit bacterial dissemination during Escherichia coli peritonitis, Crit. Care Med. 38 (2010) 1414-1422.
96. van Zoelen et al. Receptor for advanced glycation end products facilitates host defense during Escherichia coli-induced abdominal sepsis in mice. The Journal of infectious diseases 200: 765-773, 2009.
97. Warboys et al. Role of NADPH oxidase in retinal microvascular permeability increase by RAGE activation. Investigative ophthalmology & visual science 50: 1319-1328, 2009.
98. Wautier et al. Activation of NADPH oxidase by AGE links oxidant stress to altered gene expression via RAGE. American journal of physiology 280: E685-694, 2001.
99. Xie et al. Hexameric calgranulin C (S100A12) binds to the receptor for advanced glycated end products (RAGE) using symmetric hydrophobic target-binding patches, J. Biol. Chem. 282 (2007) 4218-4231.
100. Yamada et al. The expression of advanced glycation endproduct receptors in rpe cells associated with basal deposits in human maculas. Experimental eye research 82: 840-848, 2006.
101. Yamagishi, S. “Advanced Glycation end products and receptor-oxidaive stress system in diabetic vascular complications” Therapeutic Apheresis and Dialysis 2009. V. 13 p. 534-539
102. Yang at al. High mobility group box-1 protein induces the migration and activation of human dendritic cells and acts as an alarmin, J. Leukoc. Biol. 81 (2007) 59-66.
103. Yang et al. Reversing established sepsis with antagonists of endogenous high-mobility group box 1, Proc. Natl. Acad. Sci. U.S.A. 101 (2004) 296-301.
104. Yin et al. Classical pathway complement activation on human endothelial cells. Molecular immunology 44: 2228-2234, 2007.
105. Yoshida et al. The potential role of amyloid beta in the pathogenesis of age-related macular degeneration. The Journal of clinical investigation 115: 2793-2800, 2005.
106. Zeng et al. Receptor for advanced glycation end product (RAGE)-dependent modulation of early growth response-1 in hepatic ischemia/reperfusion injury, J. Hepatol. 50 (2009) 929-936.
107. Zhou et al. Complement activation by bisretinoid constituents of RPE lipofuscin. Investigative ophthalmology & visual science 50: 1392-1399, 2009.
108. Zutter and Edelson, The alpha2beta1 integrin: a novel collectin/C1q receptor, Immunobiology 212 (2007) 343-353.

1. A method of identifying a compound capable of inhibiting binding of receptor for advanced glycation endproduct (RAGE) with complement C1q (C1q) (RAGE-C1q Binding) comprising:
a) providing an amount of the compound to be tested;
b) contacting the amount of the compound with
i) an amount of C1q, and then combining the amount of C1q with an amount of RAGE under conditions that permit RAGE-C1q binding,
ii) an amount of RAGE, and then combining the amount of RAGE with an amount of C1q under conditions that permit RAGE-C1q binding, or
iii) a mixture of an amount of C1q and an amount of RAGE under conditions that permit RAGE-C1q binding;
c) measuring the amount of RAGE-C1q binding in step b);
d) comparing the amount of RAGE-C1q binding measured in step c) with the amount of RAGE-C1q binding measured under corresponding conditions in the absence of the compound; and
e) identifying the compound as capable of inhibiting RAGE-C1q binding if the amount of RAGE-C1q binding measured in step c) is less than the amount of RAGE-C1q binding in the absence of the compound under corresponding conditions.

2. The method of claim 1, wherein in step b) C1q is immobilized and/or RAGE is immobilized on a solid matrix.

3. (canceled)

4. The method of claim 1, wherein RAGE is sRAGE.

5. The method of claim 1, wherein RAGE is on the surface of a cell.

6. The method of claim 1, wherein the amount of RAGE-C1q binding is measured by differential centrifugation, chromatography, gel filtration chromatography, ion-exchange chromatography, electrophoresis, immunoprecipitation, pulldown assay, ELISA assay, fluorescence energy transfer, surface plasmon resonance, dot blot, or in vitro tubulin deacetylation assay.

7-8. (canceled)

9. The method of claim 1, wherein the compound is an antibody, aptamer, a peptide, a small molecule, siRNA or shRNA.

10. (canceled)

11. A method of identifying a compound capable of increasing RAGE-C1q binding comprising:
a) providing an amount of the compound to be tested;
b) contacting the amount of the compound with
i) an amount of C1q, and then combining the amount of C1q with an amount of RAGE under conditions that permit RAGE-C1q binding,
ii) an amount of RAGE, and then combining the amount of RAGE with an amount of C1q under conditions that permit RAGE-C1q binding, or
iii) a mixture of an amount of C1q and an amount of RAGE under conditions that permit RAGE-C1q binding;
c) measuring the amount of RAGE-C1q binding in step b);
d) comparing the amount of RAGE-C1q binding measured in step c) with the amount of RAGE-C1q binding measured under corresponding conditions in the absence of the compound; and
e) identifying the compound as capable of increasing RAGE-C1q binding if the amount of RAGE-C1q binding measured in step c) is more than the amount of RAGE-C1q binding in the absence of the compound under corresponding conditions.

12. The method of claim 11, wherein in step b) C1q is immobilized on a solid matrix and/or RAGE is immobilized.

13-20. (canceled)

21. A method of reducing phagocytosis by a phagocyte comprising contacting the phagocyte with an effective amount of a compound capable of inhibiting RAGE-C1q binding, thereby reducing phagocytosis by the phagocyte.

22. The method of claim 21, wherein the compound capable of inhibiting RAGE-C1q binding blocks RAGE's binding sites, binding blocks C1q's binding sites, blocks C1q's globular head, or is a RAGE antigen.

23-25. (canceled)

26. The method of claim 21, wherein the compound capable of inhibiting RAGE-C1q binding is an antibody, aptamer, a peptide, a small molecule, siRNA or shRNA.

27. The method of claim 26, wherein the antibody is a RAGE antibody.

28. (canceled)

29. The method of claim 21, wherein the compound capable of inhibiting RAGE-C1q binding is sRAGE.

30-31. (canceled)

32. A method of treating a subject suffering from a disease associated with an increase or a decrease of RAGE expression comprising administering to the subject a therapeutically effective amount of a compound capable of inhibiting or increasing RAGE-C1q binding so as to thereby treat the subject.

33. The method of claim 32, wherein the disease causes inflammation.

34. The method of claim 32, wherein the disease is Age-related macular degeneration (AMD).

35. The method of claim 32, wherein the compound capable of inhibiting or increasing RAGE-C1q binding is an antibody, aptamer, a peptide, a small molecule, siRNA or and shRNA.

36-37. (canceled)

38. A method of treating a subject in need of treatment of an inflammatory disease, comprising administering to the subject a therapeutically effective amount of a compound capable of inhibiting or increasing RAGE-C1q binding on the surface of white blood cells (leukocytes) present in the subject, thereby treating the subject.

39. (canceled)

40. The method of claim 38, wherein the inflammatory disease is symtemic lupus erythematosus (SLE).

41. A method of treating a subject in need of treatment of a disease associated excess apoptosis or reduced apoptosis, comprising administering to the subject a therapeutically effective amount of a compound capable of inhibiting or increasing RAGE-C1q binding on the surface of white blood cells (leukocytes) present in the subject, thereby treating the subject.

42. (canceled)

43. The method of claim 41, wherein the disease is symtemic lupus erythematosus (SLE)

44. A method of strengthening an immune system response against a bacterial infection and bacterial cells, comprising increasing RAGE-C1q binding between 1) C1q bound to bacterial cells and 2) RAGE, thereby strengthening the immune system response against bacterial infection and bacterial cells.

45-46. (canceled)

47. A method of identifying a compound as a modulator of RAGE-C1q binding comprising
a) providing an amount of the compound to be tested;
b) contacting the amount of the compound with
i.) an amount of C1q, and then combining the amount of C1q with an amount of RAGE under conditions that permit RAGE-C1q binding,
ii.) an amount of RAGE, and then combining the amount of RAGE with an amount of C1q under conditions that permit RAGE-C1q binding, or
iii.) a mixture of an amount of C1q and an amount of RAGE under conditions that permit RAGE-C1q binding;
c) measuring the amount of RAGE-C1q binding in step b);
d) comparing the amount of RAGE-C1q binding measured in step c) with the amount of RAGE-C1q binding measured under corresponding conditions in the absence of the compound; and
e) identifying the compound as a modulator of RAGE-C1q binding, if the amount of RAGE-C1q binding measured in step c) is different than the amount of RAGE-C1q binding in the absence of the compound under corresponding conditions.

48-54. (canceled)


SEQUENCE LISTING (14 sequences)

SEQ ID NO: 1 - protein, 30 aa, Homo sapiens
AQNITARIGEPLVLKCKGAPKKPPQRLEWK

SEQ ID NO: 2 - protein, 30 aa, Unknown
  Animal of the family Murinae.
GQNITARIGEPLVLSCKGAPKKPPQQLEWK

SEQ ID NO: 3 - protein, 30 aa, Unknown
  Animal of the family Muroidea.
GQNITARIGEPLMLSCKAAPKKPTQKLEWK

SEQ ID NO: 4 - protein, 30 aa, Unknown
  Animal of the family bovinae.
DQNITARIGKPLVLNCKGAPKKPPQQLEWK

SEQ ID NO: 5 - protein, 50 aa, Homo sapiens
EQYNSTYRVVSVLTVLHQDWLNGKEYKCKVSNKALPAPIEKTISKAKGQP

SEQ ID NO: 6 - protein, 38 aa, Homo sapiens
AQNITARIGEPLVLKCKGAPKKPPQRLEWKLNTGRTEA

SEQ ID NO: 7 - protein, 38 aa, Pan troglodytes; identical to SEQ ID NO: 6

SEQ ID NO: 8 - protein, 38 aa, Macaca mulatta
AQNITARIGEPLVLKCKGAPKKPPQQLEWKLNTGRTEA

SEQ ID NO: 9 - protein, 38 aa, Mus musculus
GQNITARIGEPLVLSCKGAPKKPPQQLEWKLNTGRTEA

SEQ ID NO: 10 - protein, 38 aa, Rattus norvegicus
GQNITARIGEPLMLSCKGAPKKPTQKLEWKLNTGRTEA

SEQ ID NO: 11 - protein, 38 aa, Bos taurus
DQNITARIGKPLVLNCKGAPKKPPQQLEWKLNTGRTEA

SEQ ID NO: 12 - protein, 38 aa, Unknown; identical to SEQ ID NO: 11
  Aminal of the family Sus.

SEQ ID NO: 13 - protein, 38 aa, Canis lupus familiaris
DRNITARIGKPLVLNCRGAPKKPPQQLEWKLNTGRTEA

SEQ ID NO: 14 - protein, 38 aa, Monodelphis domestica
SQNISSRIGEPLVLNCKGAPRKPPQQLEWKLNTRRTEA